SEQUENCE LISTING (1 sequence)

SEQ ID NO: 1 - protein, 15 aa, Artificial Sequence
  Phosphorylated ′SAMS′ peptide (phosphorylated on the serine residue)
HMRSAMSGLHLVKRR

(19) United States
(12) Patent Application Publication
(10) Pub. No.: US 2023/0322815 A1
(43) Pub. Date: Oct. 12, 2023

(54) NOVEL COMPOUNDS AND THERAPEUTIC USES THEREOF

(71) Applicant: University of Huddersfield, Huddersfield (GB)

(72) Inventors: Simon John Allison, Huddersfield (GB); Roger Mortimer Phillips, Huddersfield (GB); Craig Raymond Rice, Huddersfield (GB)

(21) Appl. No.: 18/020,790

(22) PCT Filed: Aug. 10, 2021
(86) PCT No.: PCT/GB2021/052070

§ 371 Date: Feb. 10, 2023

(30) Foreign Application Priority Data
Aug. 11, 2020 (GB) 2012482.2

(51) Int. Cl. C07F 3/06; C07F 13/00; C07F 1/08; A61P 35/04

(52) U.S. Cl. CPC C07F 3/06; C07F 13/005; C07F 1/08; A61P 35/04

(57) ABSTRACT

The present invention relates to compounds of Formula I:

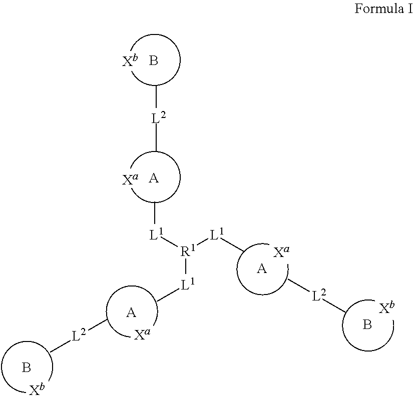

wherein R¹, L¹, A, X^{a}, L², B and X^{b} are each as defined herein. The present invention also relates to compounds of Formula II and III defined herein, formed by self-assembly of the compounds of Formula I with a metal M and an anion Q. Compounds of Formula II and III are useful in the treatment of proliferative disorders, such as cancer. The present invention also relates to pharmaceutical compositions comprising compounds of Formula I, II or III, and to the use of these compounds and compositions in the treatment of proliferative disorders, such as cancer.

### INTRODUCTION

The present invention relates to novel compounds that are useful in the treatment of proliferative disorders, such as cancer. More particularly, the invention relates to novel compounds in the form of ligands and complexes comprising the ligands. The ligands demonstrate the ability to self-assemble with certain metals and anions to form the complexes. The invention also relates to therapeutic uses of the compounds of the invention, in particular for the treatment of a proliferative disorder such as cancer.

### BACKGROUND OF THE INVENTION

Contemporary medicines range from the relatively simple such as lithium salts for the treatment of bipolar disorder, to large and complex organic structures for cancer therapy, which can contain hundreds of atoms and dozens of chiral centres.¹ However, despite their large diversity they all share a commonality; viz all are discrete molecular species that have to be chemically prepared often via an iterative synthetic procedure. A different approach to this is the use of self-assembly, which is a process where a disordered system of pre-existing compounds forms an organized structure as a consequence of specific pre-programmed interactions among the components themselves.^{2, 3, 4, 5, 6, 7, 8, 9} Self-assembly offers easy and rapid access to a library of molecularly complex architectures and novel compounds all of which may have differing biological activity without the need for a chemist to conventionally synthesise the differing molecules.

To date artificial self-assembling systems have received relatively little attention for potential therapeutic applications despite self-assembly becoming a mature area of scientific study.^{10, 11, 12, 13, 14} However, there are some notable advances in this area, for example Hannon has shown that an Fe(II)-containing dinuclear triple helicate (e.g. [L₃Fe₂]⁴⁺) interacts strongly with duplex DNA, binding in the major groove and displays therapeutic properties.^{15, 16, 17}. Other Fe(II)-containing examples include Scott and co-workers “head-to-head-to-tail” helicates which show in vitro cytotoxic activity against a range of cancer cell lines with IC₅₀ values lower than cis-platin.¹⁸ Ruthenium containing transition metal helicates and mesocates have also been shown to have interesting biological properties with some showing higher cytotoxicity towards cancer cells that lack p53.¹⁹ Metallacycles and metallacages have also been shown to possess useful properties with Han's [Pd₂L₄]⁴⁺ cages showing cytotoxicity towards an array of different human cancer cell lines.²⁰ Unsurprisingly, many of these active assemblies contain Pt²⁺ as its cytotoxic effects are well known and these have been shown to have activity towards a number of cancer cell lines, with some assemblies used to encapsulate and target cis-platin delivery.²¹

In spite of the advances discussed above, there remains a need for new compounds useful in the treatment of proliferative disorders, in particular cancer.

The present invention was devised with the foregoing in mind.

### SUMMARY OF THE INVENTION

The inventors have surprisingly discovered that compounds of Formula I described herein function as tripodal ligands that are able to self-assemble in the presence of certain metals and anions to form mono- and trinuclear complexes (the compounds of Formula II and III described herein). Interestingly, the complexes demonstrate phosphatase activity, whereby encapsulated organo-phosphate dianions (i.e. ROPO₃²⁻) are readily hydrolysed to phosphate (i.e. PO₄³⁻).

In cell studies, the complexes are highly toxic to a range of human cancer cell lines as well as a glioblastoma cancer stem cell model.^{22,23} As well as potency, the complexes show remarkable selective activity towards cancer cells compared to healthy, non-cancerous cells (by up to 2000 fold; ARPE19,²⁴ MCF10a,²⁵ and NP1^{22,23} non-cancer cell models). Encapsulation of certain anions (notably phosphate or sulfate anions) further modulates potency and selectivity.

Mechanism of action studies show that the complexes have selective phosphatase activity towards phospho-serine, phospho-tyrosine and phospho-threonine amino acids resulting in the selective inhibition of multiple kinases, cancer cell ATP depletion, autophagy and cancer cell toxicity. Kinase inhibition by certain complexes is postulated to occur through binding to phospho-amino acids rather than dephosphorylation. These represent highly novel mechanisms of action with the ability to target multiple kinases whilst retaining selective cancer cell toxicity a feature that would be predicted to suppress the emergence of resistant cancer cell sub-populations.

Thus, according to a first aspect of the present invention there is provided a compound having a structure according to Formula I as defined herein, or a pharmaceutically acceptable salt, hydrate or solvate thereof, with the proviso that the compound is not Compound Ia defined herein or a pharmaceutically acceptable salt, hydrate or solvate thereof.

According to a further aspect of the present invention there is provided a compound having a structure according to Formula II as defined herein, or a pharmaceutically acceptable salt, hydrate or solvate thereof. Suitably, the compound not Compound IIa defined herein or a pharmaceutically acceptable salt, hydrate or solvate thereof. More suitably, M is selected from the group consisting of Zn²⁺, Mn²⁺, Fe²⁺, CO²⁺ and Ni²⁺.

According to a further aspect of the present invention there is provided a compound having a structure according to Formula III as defined herein, or a pharmaceutically acceptable salt, hydrate or solvate thereof, with the proviso that the compound does not have a structure according to Formula IIIa defined herein. Suitably, M is selected from the group consisting of Zn²⁺, Mn²⁺, Fe²⁺, CO²⁺ and Ni²⁺.

According to a further aspect of the present invention there is provided a pharmaceutical composition comprising a compound having a structure according to Formula I as defined herein, or a pharmaceutically acceptable salt, hydrate or solvate thereof, a source of M as defined herein, and one or more pharmaceutically acceptable diluents, excipients or carriers. Suitably, the pharmaceutical composition further comprises a source of Q as defined herein.

According to a further aspect of the present invention there is provided a pharmaceutical composition comprising a compound having a structure according to Formula II as defined herein, or a pharmaceutically acceptable salt, hydrate or solvate thereof, and one or more pharmaceutically acceptable diluents, excipients or carriers. Suitably, the pharmaceutical composition further comprises a source of Q as defined herein.

According to a further aspect of the present invention there is provided a pharmaceutical composition comprising a compound having a structure according to Formula III as defined herein or a pharmaceutically acceptable salt, hydrate or solvate thereof, and one or more pharmaceutically acceptable diluents, excipients or carriers.

According to a further aspect of the present invention there is provided a kit of parts comprising a compound having a structure according to Formula I as defined herein, or a pharmaceutically acceptable salt, hydrate or solvate thereof, and a source of M as defined herein.

According to a further aspect of the present invention there is provided a kit of parts comprising a compound having a structure according to Formula II as defined herein, or a pharmaceutically acceptable salt, hydrate or solvate thereof, and a source of Q as defined herein.

According to a further aspect of the present invention there is provided a compound of Formula I as defined herein, or a pharmaceutically acceptable salt, hydrate or solvate thereof, in combination with a source of M as defined herein, for use as a medicament. Suitably, the compound of Formula I and the source of M are in further combination with a source of Q as defined herein.

According to a further aspect of the present invention there is provided a compound of Formula II as defined herein, or a pharmaceutically acceptable salt, hydrate or solvate thereof, for use as a medicament. Suitably, the compound of Formula II is in combination with a source of Q as defined herein.

According to a further aspect of the present invention there is provided a compound of Formula III as defined herein, or a pharmaceutically acceptable salt, hydrate or solvate thereof, for use as a medicament.

According to a further aspect of the present invention there is provided a pharmaceutical composition as defined herein, or a pharmaceutically acceptable salt, hydrate or solvate thereof, for use as a medicament.

According to a further aspect of the present invention there is provided a compound of Formula I as defined herein, or a pharmaceutically acceptable salt, hydrate or solvate thereof, in combination with a source of M as defined herein, for use in the treatment of a proliferative disorder (e.g. cancer). Suitably, the compound of Formula I and the source of M are in further combination with a source of Q as defined herein.

According to a further aspect of the present invention there is provided a compound of Formula II as defined herein, or a pharmaceutically acceptable salt, hydrate or solvate thereof, for use in the treatment of a proliferative disorder (e.g. cancer). Suitably, the compound of Formula II is in combination with a source of Q as defined herein.

According to a further aspect of the present invention there is provided a compound of Formula III as defined herein, or a pharmaceutically acceptable salt, hydrate or solvate thereof, for use in the treatment of a proliferative disorder (e.g. cancer).

According to a further aspect of the present invention there is provided a pharmaceutical composition as defined herein, or a pharmaceutically acceptable salt, hydrate or solvate thereof, for use in the treatment of a proliferative disorder (e.g. cancer).

According to a further aspect of the present invention there is provided a method of treating a proliferative disorder (e.g. cancer) in a patient in need of such treatment, the method comprising administering to the patient a therapeutically effective amount of a compound of Formula I as defined herein, or a pharmaceutically acceptable salt, hydrate or solvate thereof, in combination with a source of M as defined herein. Suitably, the compound of Formula I and the source of M are administered in combination with a source of Q.

According to a further aspect of the present invention there is provided a method of treating a proliferative disorder (e.g. cancer) in a patient in need of such treatment, the method comprising administering to the patient a therapeutically effective amount of a compound of Formula II as defined herein, or a pharmaceutically acceptable salt, hydrate or solvate thereof. Suitably, the compound of Formula II is administered in combination with a source of Q.

According to a further aspect of the present invention there is provided a method of treating a proliferative disorder (e.g. cancer) in a patient in need of such treatment, the method comprising administering to the patient a therapeutically effective amount of a compound of Formula III as defined herein, or a pharmaceutically acceptable salt, hydrate or solvate thereof.

According to a further aspect of the present invention there is provided a method of treating a proliferative disorder (e.g. cancer) in a patient in need of such treatment, the method comprising administering to the patient a therapeutically effective amount of a pharmaceutical composition as defined herein.

According to a further aspect of the present invention there is provided the use of a compound of Formula I, II or III, or a pharmaceutically acceptable salt, hydrate or solvate thereof, in the manufacture of a medicament. Suitably, the medicament is for the treatment of a proliferative disorder (e.g. cancer).

In the above-outlined aspects of the invention, the proliferative disorder is suitably cancer, and the cancer is suitably a human cancer. In particular, the compounds of the present invention will be useful for the treatment of any cancer in which a mis-match repair inhibition is beneficial.

Any suitable cancer may be targeted (e.g. adenoid cystic carcinoma, adrenal gland tumor, amyloidosis, anal cancer, appendix cancer, astrocytoma, ataxia-telangiectasia, Beckwith-Wiedemann Syndrome, bile duct cancer (cholangiocarcinoma), Birt-Hogg-Dubé Syndrome, bladder cancer, bone cancer, brain stem glioma, brain tumor, breast cancer, Carney Complex, central nervous system tumors, cervical cancer, colorectal cancer, Cowden Syndrome, craniopharyngioma, desmoplastic infantile ganglioglioma, ependymoma, esophageal cancer, Ewing sarcoma, eye cancer, eyelid cancer, familial adenomatous polyposis, familial GIST, familial malignant melanoma, familial non-VHL clear cell renal cell carcinoma, familial pancreatic cancer, gallbladder cancer, gastrointestinal stromal tumor—GIST, germ cell tumor, gestational trophoblastic disease, head and neck cancer, hereditary breast and ovarian cancer, hereditary diffuse gastric cancer, hereditary leiomyomatosis and renal cell cancer, hereditary mixed polyposis syndrome, hereditary pancreatitis, hereditary papillary renal carcinoma, juvenile polyposis syndrome, kidney cancer, lacrimal gland tumor, laryngeal and hypopharyngeal cancer, leukemia (acute lymphoblastic leukamia (ALL), acute myeloid leukemia (AML), B-cell prolymphocytic leukemia, hairy cell leukemia, chronic lymphocytic leukemia (CLL), chronic myeloid leukemia (CML), chronic T-cell lymphocytic leukemia, eosinophilic leukemia), Li-Fraumeni Syndrome, liver cancer, lung cancer (non-small cell lung cancer, small cell lung cancer), Lymphoma (Hodgkin, non-Hodgkin), Lynch Syndrome, mastocytosis, medulloblastoma, melanoma, meningioma, mesothelioma, multiple endocrine neoplasia Type 1 & 2, multiple myeloma, MUTYH (or MYH)-associated polyposis, myelodysplastic syndromes (MDS), nasal cavity and paranasal sinus Cancer, nasopharyngeal Cancer, neuroblastoma, neuroendocrine tumors (e.g. of the gastrointestinal tract, lung or pancreas), neurofibromatosis Type 1 & 2, nevoid basal cell carcinoma syndrome, oral and oropharyngeal cancer, osteosarcoma, ovarian/fallopian tube/peritoneal cancer, pancreatic cancer, parathyroid cancer, penile cancer, Peutz-Jeghers Syndrome, pheochromocytoma, paraganglioma, pituitary gland tumor, pleuropulmonary blastoma, prostate cancer, retinoblastoma, rhabdomyosarcoma, salivary gland cancer, sarcoma (e.g. Kaposi or soft tissue), skin cancer, small bowel cancer, stomach cancer, testicular cancer, thymoma and thymic carcinoma, thyroid cancer, tuberous sclerosis complex, uterine cancer, vaginal cancer, Von Hippel-Lindau syndrome, vulvar cancer, Waldenstrom's macroglobulinemia, Werner syndrome, Wilms Tumor and xeroderma pigmentosum). Particular cancers of interest include haematological cancers such as lymphomas (including diffuse large B-cell lymphoma (DLBCL), follicular lymphoma (FL), Burkitt lymphoma (BL) and angioimmunoblastic T-cell lymphoma (AITL)), leukaemias (including acute lymphoblastic leukaemia (ALL) and chronic myeloid leukaemia (CML)), multiple myeloma, breast cancer, non-small cell lung cancer (NSCLC), colorectal cancer, endometrial cancer, gastro-oesophageal cancer, neuroendocrine cancers, osteosarcomas, prostate cancer, pancreatic cancer, small intestine cancer, bladder cancer, rectal cancer, cholangiocarcinoma, CNS cancer, thyroid cancer, head and neck cancer, oesophageal cancer, and ovarian cancer.

Components used in combination with one another (e.g. the compounds of Formula I, II or III, and sources of M and Q) may be administered simultaneously, separately or sequentially. In one aspect of the invention in combination with refers to simultaneous administration. In another aspect of the invention in combination with refers to separate administration. In a further aspect of the invention in combination with refers to sequential administration. Where the administration is sequential or separate, the delay in administering the second (and any subsequent) component should not be such as to lose the beneficial effect of the combination.

Features, including optional, suitable, and preferred features in relation to one aspect of the invention may also be features, including optional, suitable and preferred features in relation to any other aspect of the invention.

### DETAILED DESCRIPTION OF THE INVENTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated. It should be understood that in, for example, a human or other mammal, a therapeutically effective amount can be determined experimentally in a laboratory or clinical setting, or a therapeutically effective amount may be the amount required by the guidelines of the United States Food and Drug Administration (FDA) or equivalent foreign regulatory body, for the particular disease and subject being treated. It should be appreciated that determination of proper dosage forms, dosage amounts, and routes of administration is within the level of ordinary skill in the pharmaceutical and medical arts.

As used herein by themselves or in conjunction with another term or terms, “subject(s)” and “patient(s)”, refer to animals (e.g. mammals), particularly humans. Suitably, the “subject(s)” and “patient(s)” may be a non-human animal (e.g. livestock and domestic pets) or a human.

As used herein by itself or in conjunction with another term or terms, “pharmaceutically acceptable” refers to materials that are generally chemically and/or physically compatible with other ingredients (such as, for example, with reference to a formulation), and/or is generally physiologically compatible with the recipient (such as, for example, a subject) thereof.

In this specification the term “alkyl” includes both straight and branched chain alkyl groups. References to individual alkyl groups such as “propyl” are specific for the straight chain version only and references to individual branched chain alkyl groups such as “isopropyl” are specific for the branched chain version only. For example, “(1-6C)alkyl” includes (1-4C)alkyl, (1-3C)alkyl, propyl, isopropyl and t-butyl.

The term “(m-nC)” or “(m-nC) group” used alone or as a prefix, refers to any group having m to n carbon atoms.

An “alkylene” group is an alkyl group that is positioned between and serves to connect two other chemical groups. Thus, “(1-6C)alkylene” means a linear saturated divalent hydrocarbon radical of one to six carbon atoms or a branched saturated divalent hydrocarbon radical of three to six carbon atoms, for example, methylene (—CH₂—), the ethylene isomers (—CH(CH₃)— and —CH₂CH₂—), the propylene isomers (—CH(CH₃)CH₂—, —CH(CH₂CH₃)—, —C(CH₃)₂—, and —CH₂CH₂CH₂—), pentylene (—CH₂CH₂CH₂CH₂CH₂—), and the like.

The term “alkyenyl” refers to straight and branched chain alkyl groups comprising 2 or more carbon atoms, wherein at least one carbon-carbon double bond is present within the group. Examples of alkenyl groups include ethenyl, propenyl and but-2,3-enyl and includes all possible geometric (E/Z) isomers.

The term “alkynyl” refers to straight and branched chain alkyl groups comprising 2 or more carbon atoms, wherein at least one carbon-carbon triple bond is present within the group. Examples of alkynyl groups include acetylenyl and propynyl.

“(m-nC)cycloalkyl” means a saturated hydrocarbon ring system containing from m to n number of carbon atoms. Exemplary cycloalkyl groups include, for example, cyclopropyl, cyclobutyl, cyclopentyl, cyclohexyl, cycloheptyl and bicyclo[2.2.1]heptyl.

The term “alkoxy” refers to O-linked straight and branched chain alkyl groups. Examples of alkoxy groups include methoxy, ethoxy and t-butoxy.

The term “haloalkyl” is used herein to refer to an alkyl group in which one or more hydrogen atoms have been replaced by halogen (e.g. fluorine) atoms. Often, haloalkyl is fluoroalkyl. Examples of haloalkyl groups include —CH₂F, —CHF₂ and —CF₃.

The term “halo” or “halogeno” refers to fluoro, chloro, bromo and iodo, suitably fluoro, chloro and bromo, more suitably, fluoro and chloro.

The term “carbocyclyl”, “carbocyclic” or “carbocycle” means a non-aromatic saturated or partially saturated monocyclic, fused, bridged, or spiro bicyclic carbon-containing ring system(s). Monocyclic carbocyclic rings contain from about 3 to 12 (suitably from 3 to 7) ring atoms. Bicyclic carbocycles contain from 6 to 17 member atoms, suitably 7 to 12 member atoms, in the ring. Bicyclic carbocyclic(s) rings may be fused, spiro, or bridged ring systems. Examples of carbocyclic groups include cyclopropyl, cyclobutyl, cyclohexyl, cyclohexenyl and spiro[3.3]heptanyl.

The term “heterocyclyl”, “heterocyclic” or “heterocycle” means a non-aromatic saturated or partially saturated monocyclic, fused, bridged, or spiro bicyclic heterocyclic ring system(s). Monocyclic heterocyclic rings contain from about 3 to 12 (suitably from 3 to 7) ring atoms, with from 1 to 5 (suitably 1, 2 or 3) heteroatoms selected from nitrogen, oxygen or sulfur in the ring. Bicyclic heterocycles contain from 7 to 17 member atoms, suitably 7 to 12 member atoms, in the ring. Bicyclic heterocyclic(s) rings may be fused, spiro, or bridged ring systems. Examples of heterocyclic groups include cyclic ethers such as oxiranyl, oxetanyl, tetrahydrofuranyl, dioxanyl, and substituted cyclic ethers. Heterocycles containing nitrogen include, for example, azetidinyl, pyrrolidinyl, piperidinyl, piperazinyl, tetrahydrotriazinyl, tetrahydropyrazolyl, and the like. Typical sulfur containing heterocycles include tetrahydrothienyl, dihydro-1,3-dithiol, tetrahydro-2H-thiopyran, and hexahydrothiepine. Other heterocycles include dihydro-oxathiolyl, tetrahydro-oxazolyl, tetrahydro-oxadiazolyl, tetrahydrodioxazolyl, tetrahydro-oxathiazolyl, hexahydrotriazinyl, tetrahydro-oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as tetrahydrothiene 1,1-dioxide and thiomorpholinyl 1,1-dioxide. Heterocycles may comprise 1 or 2 oxo (═O) or thioxo (═S) substituents. A suitable value for a heterocyclyl group which bears 1 or 2 oxo (═O) or thioxo (═S) substituents is, for example, 2-oxopyrrolidinyl, 2-thioxopyrrolidinyl, 2-oxoimidazolidinyl, 2-thioxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a carbon or nitrogen atom. However, reference herein to piperidino or morpholino refers to a piperidin-1-yl or morpholin-4-yl ring that is linked via the ring nitrogen.

By “bridged ring systems” is meant ring systems in which two rings share more than two atoms, see for example Advanced Organic Chemistry, by Jerry March, 4^{th} Edition, Wiley Interscience, pages 131-133, 1992. Examples of bridged heterocyclyl ring systems include, aza-bicyclo[2.2.1]heptane, 2-oxa-5-azabicyclo[2.2.1]heptane, aza-bicyclo[2.2.2]octane, aza-bicyclo[3.2.1]octane and quinuclidine.

By “spiro bi-cyclic ring systems”, it is meant that the two ring systems share one common spiro carbon atom, i.e. the heterocyclic ring is linked to a further carbocyclic or heterocyclic ring through a single common spiro carbon atom. Examples of spiro ring systems include 6-azaspiro[3.4]octane, 2-oxa-6-azaspiro[3.4]octane, 2-azaspiro[3.3]heptanes, 2-oxa-6-azaspiro[3.3]heptanes, 7-oxa-2-azaspiro[3.5]nonane, 6-oxa-2-azaspiro[3.4]octane, 2-oxa-7-azaspiro[3.5]nonane and 2-oxa-6-azaspiro[3.5]nonane.

As used herein by itself or in conjunction with another term or terms, “aromatic” refers to monocyclic and polycyclic ring systems containing 4n+2 pi electrons, where n is an integer. Aromatic should be understood as referring to and including ring systems that contain only carbon atoms (i.e. “aryl”) as well as ring systems that contain at least one heteroatom selected from N, O or S (i.e. “heteroaromatic” or “heteroaryl”). An aromatic ring system can be substituted or unsubstituted.

As used herein by itself or in conjunction with another term or terms, “non-aromatic” refers to a monocyclic or polycyclic ring system having at least one double bond that is not part of an extended conjugated pi system. As used herein, non-aromatic refers to and includes ring systems that contain only carbon atoms as well as ring systems that contain at least one heteroatom selected from N, O or S. A non-aromatic ring system can be substituted or unsubstituted.

The term “aryl” means a cyclic or polycyclic aromatic ring having from 5 to 12 carbon atoms. The term aryl includes both monovalent species and divalent species. Examples of aryl groups include, but are not limited to, phenyl, biphenyl, naphthyl and the like. In a particular embodiment, an aryl is phenyl.

The term “heteroaryl” or “heteroaromatic” means an aromatic mono-, bi-, or polycyclic ring incorporating one or more (for example 1-4, particularly 1, 2 or 3) heteroatoms selected from nitrogen, oxygen or sulfur. The term heteroaryl includes both monovalent species and divalent species. Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 5H-pyrido[2,3-d]-o-oxazinyl, 1H-pyrazolo[4,3-d]-oxazolyl, 4H-imidazo[4,5-d]thiazolyl, pyrazino[2,3-d]pyridazinyl, imidazo[2,1-b]thiazolyl, imidazo[1,2-b][1,2,4]triazinyl. “Heteroaryl” also covers partially aromatic bi- or polycyclic ring systems wherein at least one ring is an aromatic ring and one or more of the other ring(s) is a non-aromatic, saturated or partially saturated ring, provided at least one ring contains one or more heteroatoms selected from nitrogen, oxygen or sulfur. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl and 3,4-dihydro-2H-pyrido[3,2-b][1,4]oxazinyl.

Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six membered heteroaryl groups include but are not limited to pyridyl, pyrazinyl, pyridazinyl, pyrimidinyl and triazinyl.

A bicyclic heteroaryl group may be, for example, a group selected from:

a benzene ring fused to a 5- or 6-membered ring containing 1, 2 or 3 ring heteroatoms;
a pyridine ring fused to a 5- or 6-membered ring containing 1, 2 or 3 ring heteroatoms;
a pyrimidine ring fused to a 5- or 6-membered ring containing 1 or 2 ring heteroatoms;
a pyrrole ring fused to a 5- or 6-membered ring containing 1, 2 or 3 ring heteroatoms;
a pyrazole ring fused to a 5- or 6-membered ring containing 1 or 2 ring heteroatoms;
a pyrazine ring fused to a 5- or 6-membered ring containing 1 or 2 ring heteroatoms;
an imidazole ring fused to a 5- or 6-membered ring containing 1 or 2 ring heteroatoms;
an oxazole ring fused to a 5- or 6-membered ring containing 1 or 2 ring heteroatoms;
an isoxazole ring fused to a 5- or 6-membered ring containing 1 or 2 ring heteroatoms;
a thiazole ring fused to a 5- or 6-membered ring containing 1 or 2 ring heteroatoms;
an isothiazole ring fused to a 5- or 6-membered ring containing 1 or 2 ring heteroatoms;
a thiophene ring fused to a 5- or 6-membered ring containing 1, 2 or 3 ring heteroatoms;
a furan ring fused to a 5- or 6-membered ring containing 1, 2 or 3 ring heteroatoms;
a cyclohexyl ring fused to a 5- or 6-membered heteroaromatic ring containing 1, 2 or 3 ring heteroatoms; and
a cyclopentyl ring fused to a 5- or 6-membered heteroaromatic ring containing 1, 2 or 3 ring heteroatoms.

Particular examples of bicyclic heteroaryl groups containing a six membered ring fused to a five membered ring include but are not limited to benzfuranyl, benzthiophenyl, benzimidazolyl, benzoxazolyl, benzisoxazolyl, benzthiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl and pyrazolopyridinyl groups.

Particular examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

This specification also makes use of several composite terms to describe groups comprising more than one functionality. Such terms will be understood by a person skilled in the art. For example (3-6C)cycloalkyl(m-nC)alkyl comprises (m-nC)alkyl substituted by (3-6C)cycloalkyl.

The term “optionally substituted” refers to either groups, structures, or molecules that are substituted and those that are not substituted. The term “wherein a/any CH, CH₂, CH₃ group or heteroatom (i.e. NH) within a R¹ group is optionally substituted” suitably means that (any) one of the hydrogen radicals of the R¹ group is substituted by a relevant stipulated group.

Where optional substituents are chosen from “one or more” groups it is to be understood that this definition includes all substituents being chosen from one of the specified groups or the substituents being chosen from two or more of the specified groups. In some embodiments, one or more refers to one, two or three. In another embodiment, one or more refers to one or two. In a particular embodiment, one or more refers to one.

The phrase “compound of the invention” means those compounds which are disclosed herein, both generically and specifically.

“About” when used herein in conjunction with a measurable value such as, for example, an amount or a period of time and the like, is meant to encompass reasonable variations of the value, for instance, to allow for experimental error in the measurement of said value.

### Compounds of the Invention

Unless otherwise specified, it will be understood that reference made herein to a compound of the invention refers to a compound of any one of Formula I, II or III (including all sub-formulae), or a pharmaceutically acceptable salt, hydrate or solvate thereof. Similarly, it will be understood that reference made herein to the compounds of the invention refers collectively to the compounds of Formula I, II and III (including all sub-formulae), as well as pharmaceutically acceptable salts, hydrates or solvates thereof.

### Compounds of Formula I

In one aspect, the present invention provides a compound having a structure according to Formula I shown below, or a pharmaceutically acceptable salt, hydrate or solvate thereof:

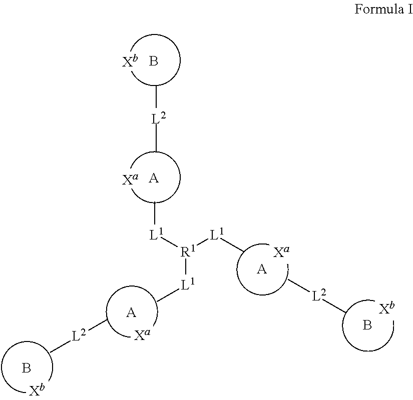

wherein
R¹ is selected from the group consisting of N, CR², aryl, heteroaryl, carbocyclyl and heterocyclyl, where any aryl, heteroaryl, carbocyclyl or heterocyclyl in R¹ is optionally substituted with one or more R³;
each R³ is independently selected from the group consisting of hydroxy, cyano, halogen, (1-4C)alkyl, (1-4C)haloalkyl, (2-4C)alkenyl, (2-4C)alkynyl, aryl, aryl(1-3C)alkyl, heteroaryl, heteroaryl(1-3C)alkyl, carbocyclyl, carbocyclyl(1-3C)alkyl, heterocyclyl, heterocyclyl(1-3C)alkyl, —OR^{3a}, —NR^{3a}R^{3b}, —C(O)—R^{3a}, —C(O)—OR^{3a}, —O—C(O)—R^{3a}, —C(O)—NR^{3a}R^{3b}, —N(R^{3a})C(O)—R^{3b} and —S(O)₀₋₂R^{3a}, where any (1-4C)alkyl, (1-4C)haloalkyl, (2-4C)alkenyl, (2-4C)alkynyl, aryl, aryl(1-3C)alkyl, heteroaryl, heteroaryl(1-3C)alkyl, carbocyclyl, carbocyclyl(1-3C)alkyl, heterocyclyl or heterocyclyl(1-3C)alkyl in R³ is optionally substituted with one or more R^{3c};
R^{3a} and R^{3b} are each independently selected from the group consisting of hydrogen, (1-3C)alkyl and (1-3C)haloalkyl;
each R^{3c} is independently selected from the group consisting of hydroxy, halogen, cyano, amino, (1-3C)alkyl, (1-3C)alkoxy and (1-3C)haloalkyl;
R² is selected from the group consisting of hydrogen, hydroxy, cyano, halogen, (1-4C)alkyl, (1-4C)haloalkyl, (2-4C)alkenyl, (2-4C)alkynyl, aryl, aryl(1-3C)alkyl, heteroaryl, heteroaryl(1-3C)alkyl, carbocyclyl, carbocyclyl(1-3C)alkyl, heterocyclyl, heterocyclyl(1-3C)alkyl, —OR^{2a}, —NR^{2a}R^{2b}, —C(O)—R^{2a}, —C(O)—OR^{2a}, —O—C(O)—R^{2a}, —C(O)—NR^{2a}R^{2b}, —N(R^{2a})C(O)—R^{2b} and —S(O)₀₋₂R^{9a}, where any (1-4C)alkyl, (1-4C)haloalkyl, (2-4C)alkenyl, (2-4C)alkynyl, aryl, aryl(1-3C)alkyl, heteroaryl, heteroaryl(1-3C)alkyl, carbocyclyl, carbocyclyl(1-3C)alkyl, heterocyclyl or heterocyclyl(1-3C)alkyl in R² is optionally substituted with one or more R^{2c};
R^{2a} and R^{2b} are each independently selected from the group consisting of hydrogen, (1-3C)alkyl and (1-3C)haloalkyl;
each R^{2c} is independently selected from the group consisting of hydroxy, halogen, cyano, amino, (1-3C)alkyl, (1-3C)alkoxy and (1-3C)haloalkyl;
each L¹ is a group:

—(W)ₙ—(X)ₘ—(Y)ₒ—(Z)ₚ—

in which
n and o are each independently 0, 1 or 2, and m and p are each independently 0 or 1, with the provisos that when m and p are both 1, o is not 0;
each W is selected from the group consisting of (1-3C)alkylene, (2-3C)alkenylene, (2-3C)alkynylene, arylene, heteroarylene, carbocyclylene and heterocyclylene, where any (1-3C)alkylene, (2-3C)alkenylene, (2-3C)alkynylene, arylene, heteroarylene, carbocyclylene or heterocyclylene in W is optionally substituted with one or more W^{a}, where each W^{a} is independently selected from the group consisting of hydroxy, cyano, halogen, amino, (1-2)alkoxy and (1-2C)haloalkyl;
X is selected from the group consisting of —O—, —C(O)—, —C(O)—O—, —O—C(O)—, —S(O)₀₋₂—, —C(O)—N(R^{x})—, —N(R^{x})—C(O)—, —NR^{x}—, —N(R^{x})—C(O)—NR^{x}—, —SO₂N(R^{x})—, and —N(R^{x})SO₂, where each R^{x} is independently selected from the group consisting of hydrogen, hydroxy, cyano, (1-4C)alkyl, (2-4C)alkenyl and (2-4C)alkynyl;
each Y is selected from the group consisting of (1-3C)alkylene, (2-3C)alkenylene, (2-3C)alkynylene, arylene, heteroarylene, carbocyclylene and heterocyclylene, where any (1-3C)alkylene, (2-3C)alkenylene, (2-3C)alkynylene, arylene, heteroarylene, carbocyclylene or heterocyclylene in Y is optionally substituted with one or more Y^{a}, where each Y^{a} is independently selected from the group consisting of hydroxy, cyano, halogen, amino, (1-2)alkoxy and (1-2C)haloalkyl;
Z is selected from the group consisting of —O—, —C(O)—, —C(O)—O—, —O—C(O)—, —S(O)₀₋₂—, —C(O)—N(R^{z})—, —N(R^{z})—C(O)—, —NR^{z}—, —N(R^{z})—C(O)—NR^{z}—, —SO₂N(R^{z})—, and —N(R^{z})SO₂, where each R^{z} is independently selected from the group consisting of hydrogen, hydroxy, cyano, (1-4C)alkyl, (2-4C)alkenyl and (2-4C)alkynyl;
X^{a} is a ring heteroatom located within ring A and is selected from N and O;
each ring A is a monocyclic heteroaryl, bicyclic heteroaryl, monocyclic heterocycle or bicyclic heterocycle, any one of which is optionally substituted with one or more R⁴, where each R⁴ is independently selected from the group consisting of hydroxy, cyano, halogen, (1-6C)alkyl, (1-6C)haloalkyl, (2-6C)alkenyl, (2-6C)alkynyl, aryl, aryl(1-3C)alkyl, heteroaryl, heteroaryl(1-3C)alkyl, carbocyclyl, carbocyclyl(1-3C)alkyl, heterocyclyl, heterocyclyl(1-3C)alkyl, —R^{4a}—OR^{4b}, —R^{4a}—NR^{4b}R^{4c}, —R^{4a}—C(O)—R^{4b}, —R^{4a}—C(O)—OR^{4b}, —R^{4a}—O—C(O)—R^{4b}, —R^{4a}—C(O)—NR^{4b}R^{4c}, —R^{4a}.
N(R^{4b})C(O)—R^{4c} and —R^{4a}—S(O)₀₋₂R^{4b}, where any (1-6C)alkyl, (1-6C)haloalkyl, (2-6C)alkenyl, (2-6C)alkynyl, aryl, aryl(1-3C)alkyl, heteroaryl, heteroaryl(1-3C)alkyl, carbocyclyl, carbocyclyl(1-3C)alkyl, heterocyclyl or heterocyclyl(1-3C)alkyl in R⁴ is optionally substituted with one or more R^{4d};
R^{4a} is absent or is (1-3C)alkylene that is optionally substituted with one or substituents selected from group consisting of hydroxy, halo and amino;
R^{4b} and R^{4c} are each independently selected from the group consisting of hydrogen, (1-3C)alkyl and (1-3C)haloalkyl;
each R^{4d} is independently selected from the group consisting of hydroxy, halogen, cyano, amino, (1-3C)alkyl, (1-3C)alkoxy and (1-3C)haloalkyl;
X^{b} is a ring heteroatom located within ring B and is selected from N and O
each ring B is a monocyclic heteroaryl, bicyclic heteroaryl, monocyclic heterocycle or bicyclic heterocycle, any one of which is optionally substituted with one or more R⁵, where each R⁵ is independently selected from the group consisting of hydroxy, cyano, halogen, (1-6C)alkyl, (1-6C)haloalkyl, (2-6C)alkenyl, (2-6C)alkynyl, aryl, aryl(1-3C)alkyl, heteroaryl, heteroaryl(1-3C)alkyl, carbocyclyl, carbocyclyl(1-3C)alkyl, heterocyclyl, heterocyclyl(1-3C)alkyl, —R^{5a}—OR^{5b}, —R^{5a}—NR^{5b}R^{5c}, —R^{5a}—C(O)—R^{5b}, —R^{5a}—C′(O)—OR^{5b}, —R^{5a}—O—C(O)—R^{5b}, —R^{5a}—C′(O)—NR^{5b}R^{5c}, —R^{5a}. N(R^{5b})C(O)—R^{5c} and —R^{5a}—S(O)₀₋₂R^{5b}, where any (1-6C)alkyl, (1-6C)haloalkyl, (2-6C)alkenyl, (2-6C)alkynyl, aryl, aryl(1-3C)alkyl, heteroaryl, heteroaryl(1-3C)alkyl, carbocyclyl, carbocyclyl(1-3C)alkyl, heterocyclyl or heterocyclyl(1-3C)alkyl in R⁵ is optionally substituted with one or more R^{5d};
R^{5a} is absent or is (1-3C)alkylene that is optionally substituted with one or substituents selected from group consisting of hydroxy, halo and amino;
R^{5b} and R^{5c} are each independently selected from the group consisting of hydrogen, (1-5)alkyl (e.g. (1-3C)alkyl) and (1-3C)haloalkyl;
each R^{5d} is independently selected from the group consisting of hydroxy, halogen, cyano, amino, (1-3C)alkyl, (1-3C)alkoxy and (1-3C)haloalkyl;
each L² is selected from the group consisting of absent (in which case ring A is bonded directly to ring B), (1-2C)alkylene, ethenylene and ethynylene, where any (1-2C)alkylene, ethenylene and ethynylene in L² is optionally substituted with one or more substituents selected form the group consisting of hydroxy, halogen, cyano, amino, (1-3C)alkyl, (1-3C)alkoxy and (1-3C)haloalkyl;
with the proviso that the compound of Formula I is not the following Compound Ia, or a pharmaceutically acceptable salt, hydrate or solvate thereof:

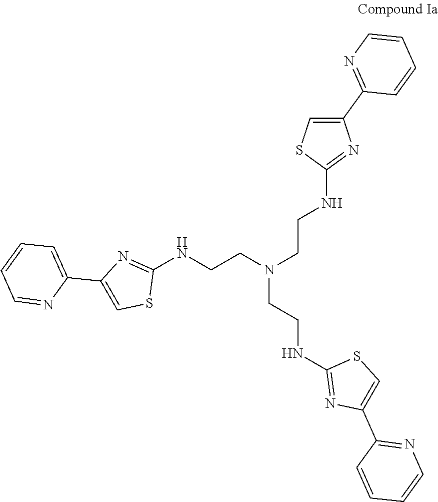 [SMILES: c1ccc(-c2csc(NCCN(CCNc3nc(-c4ccccn4)cs3)CCNc3nc(-c4ccccn4)cs3)n2)nc1]

Particular compounds of the invention include, for example, compounds of the Formula I, or pharmaceutically acceptable salts, hydrates and/or solvates thereof, wherein, unless otherwise stated, each of R¹, L¹, A, X^{a}, L², B and X^{b}, and any associated subgroup, has any of the meanings defined hereinbefore or in any of paragraphs (1) to (80) hereinafter:
(1) Each ring A is a 5-7 membered monocyclic heteroaryl containing 1, 2 or 3 ring heteroatoms in total independently selected from N, O and S, or a 5-7 membered monocyclic heterocycle containing 1, 2 or 3 ring heteroatoms in total independently selected from N, O and S, wherein each ring A is optionally substituted with one or more R⁴.
(2) Each ring A is a 5-6 membered monocyclic heteroaryl containing 1, 2 or 3 ring heteroatoms in total independently selected from N, O and S, or a 5-6 membered monocyclic heterocycle containing 1, 2 or 3 ring heteroatoms in total independently selected from N, O and S, wherein each ring A is optionally substituted with one or more R⁴.
(3) Each ring A is group:

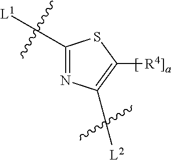

wherein a is 0 or 1.
(4) Each ring A is group:

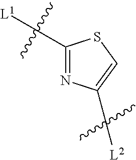

(5) X^{a} is N and ring A contains 0, 1 or 2 further ring heteroatoms selected from N, O and S.
(6) X^{a} is N and ring A contains 0 or 1 further ring heteroatoms selected from N, O and S.
(7) X^{a} is located immediately adjacent the carbon atom bonded to L¹.
(8) Each R⁴ is independently selected from the group consisting of hydroxy, halogen, (1-6C)alkyl, (1-6C)haloalkyl, (2-6C)alkenyl, phenyl, phenyl(1-3C)alkyl, 5-6 membered heteroaryl, 5-6 membered heteroaryl(1-3C)alkyl, —R^{4a}—OR^{4b}, —R^{4a}—NR^{4b}R^{4c}, —R^{4a}—C(O)—R^{4b}, —R^{4a}—C(O)—OR^{4b}, —R^{4a}—O—C(O)—R^{4b}, —R^{4a}—C(O)—NR^{4b}R^{4c}, —R^{4a}—N(R^{4b})C(O)—R^{4c} and —R^{4a}. S(O)₀₋₂R^{4b}, where any (1-6C)alkyl, (1-6C)haloalkyl, (2-6C)alkenyl, phenyl, phenyl(1-3C)alkyl, 5-6 membered heteroaryl or 5-6 membered heteroaryl(1-3C)alkyl in R⁴ is optionally substituted with one or more R^{4d}.
(9) Each R⁴ is independently selected from the group consisting of hydroxy, halogen, (1-3C)alkyl, (1-3C)haloalkyl, (2-3C)alkenyl, phenyl and —R^{4a}—OR^{4b}, where any (1-3C)alkyl, (1-3C)haloalkyl, (2-3C)alkenyl or phenyl in R⁴ is optionally substituted with one or more R^{4d}.
(10) Each R⁴ is independently selected from the group consisting of hydroxy, halogen, (1-3C)alkyl, (1-3C)haloalkyl and phenyl.
(11) Each R⁴ is independently selected from the group consisting of hydroxy, halogen, (1-2C)alkyl and (1-2C)haloalkyl.
(12) Each R^{4a} is absent or methylene.
(13) R^{4b} and R^{4c} are each independently selected from the group consisting of hydrogen, methyl and ethyl.
(14) Each R^{4d} is independently selected from the group consisting of hydroxy, halogen, amino, (1-2C)alkyl, (1-2C)alkoxy and (1-2C)haloalkyl.
(15) Each R^{4d} is independently selected from the group consisting of halogen, (1-2C)alkyl and (1-2C)haloalkyl. (16) Each ring B is:
   i) a 5-7 membered monocyclic heterocycle containing 1, 2 or 3 ring heteroatoms in total that are independently selected from N, O and S;
   ii) a 5-7 membered monocyclic heteroaryl containing 1, 2 or 3 ring heteroatoms in total that are independently selected from N, O and S;
   iii) a 8-10 membered bicyclic heterocycle containing 1, 2, 3 or 4 ring heteroatoms in total that are independently selected from N, O and S; or
   iv) a 8-10 membered bicyclic heteroaryl containing 1, 2, 3 or 4 ring heteroatoms in total that are independently selected from N, O and S, wherein any ring in B is optionally substituted with one or more R⁵.
(17) Each ring B is:
   i) a 5-6 membered monocyclic heterocycle containing 1, 2 or 3 ring heteroatoms in total that are independently selected from N, O and S;
   ii) a 5-6 membered monocyclic heteroaryl containing 1, 2 or 3 ring heteroatoms in total that are independently selected from N, O and S;
   iii) a 9-10 membered bicyclic heterocycle containing 1, 2 or 3 ring heteroatoms in total that are independently selected from N, O and S; or
   iv) a 9-10 membered bicyclic heteroaryl containing 1, 2 or 3 ring heteroatoms in total that are independently selected from N, O and S,
   wherein any ring in B is optionally substituted with one or more R⁵.
(18) Each ring B is any of the following:

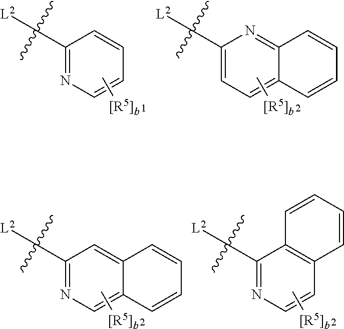

wherein
b¹ is 0, 1, 2 or 3, and
b² is 0, 1, 2, 3 or 4.
(19) Each ring B is any of the following:

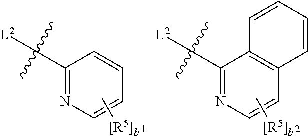

wherein
b¹ is 0, 1 or 2, and
b² is 0, 1, 2 or 3.
(20) Each ring B is any of the following:

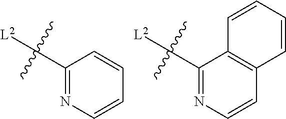

(21) Each ring B is any of the following:

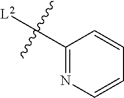

(22) X^{b} is N and ring A contains 0, 1 or 2 further ring heteroatoms selected from N, O and S.
(23) X^{b} is N and ring A contains 0 or 1 further ring heteroatoms selected from N, O and S.
(24) X^{b} is located immediately adjacent the carbon atom bonded to L².
(25) Each R⁵ is independently selected from the group consisting of hydroxy, halogen, (1-6C)alkyl, (1-6C)haloalkyl, (2-6C)alkenyl, phenyl, phenyl(1-3C)alkyl, 5-6 membered heteroaryl, 5-6 membered heteroaryl(1-3C)alkyl, —R^{5a}—OR^{5b}, —R^{5a}—NR^{5b}R^{5c}, —R^{5a}—C(O)—R^{5b}, —R^{5a}—C(O)—OR^{5b}, —R^{5a}—O—C(O)—R^{5b}, —R^{5a}—C(O)—NR^{5b}R^{5c}, —R^{5a}—N(R^{5b})C(O)—R^{5c} and —R^{5a}—S(O)₀₋₂R^{5b}, where any (1-6C)alkyl, (1-6C)haloalkyl, (2-6C)alkenyl, phenyl, phenyl(1-3C)alkyl, 5-6 membered heteroaryl or 5-6 membered heteroaryl(1-3C)alkyl in R⁵ is optionally substituted with one or more R^{5d}.
(26) Each R⁵ is independently selected from the group consisting of hydroxy, halogen, (1-6C)alkyl, (1-6C)haloalkyl, (2-6C)alkenyl, phenyl, phenyl(1-3C)alkyl, 5-6 membered heteroaryl, 5-6 membered heteroaryl(1-3C)alkyl, —R^{5a}—OR^{5b}, —R^{5a}—C(O)—R^{5b}, —R^{5a}—C(O)—OR^{5b}, —R^{5a}—O—C(O)—R^{5b}, where any (1-6C)alkyl, (1-6C)haloalkyl, (2-6C)alkenyl, phenyl, phenyl(1-3C)alkyl, 5-6 membered heteroaryl or 5-6 membered heteroaryl(1-3C)alkyl in R⁵ is optionally substituted with one or more R^{5d}.
(27) Each R⁵ is independently selected from the group consisting of hydroxy, halogen, (1-6C)alkyl, (1-6C)haloalkyl, phenyl, 5-6 membered heteroaryl, —R^{5a}—OR^{5b}, —R^{5a}—C(O)—R^{5b}, —R^{5a}—C(O)—OR^{5b}, —R^{5a}—O—C(O)—R^{5b}, where any (1-6C)alkyl, (1-6C)haloalkyl, phenyl or 5-6 membered heteroaryl in R⁵ is optionally substituted with one or more R^{5d}.
(28) Each R⁵ is independently selected from the group consisting of hydroxy, halogen, (1-3C)alkyl, (1-3C)haloalkyl, phenyl, 5-6 membered heteroaryl, —R^{5a}—OR^{5b}, —R^{5a}—C(O)—R^{5b}, —R^{5a}—C(O)—OR^{5b}, —R^{5a}—O—C(O)—R^{5b}, where any (1-3C)alkyl, (1-3C)haloalkyl, phenyl or 5-6 membered heteroaryl in R⁵ is optionally substituted with one or more R^{5d}.
(29) Each R⁵ is independently selected from the group consisting of hydroxy, halogen, (1-3C)alkyl, (1-3C)haloalkyl, —R^{5a}—OR^{5b}, —R^{5a}—C(O)—R^{5b}, —R^{5a}—C(O)—OR^{5b}, —R^{5a}—O—C(O)—R^{5b}, where any (1-3C)alkyl or (1-3C)haloalkyl in R⁵ is optionally substituted with one or more R^{5d}.
(30) Each R⁵ is independently —R^{5a}—O—C(O)—R^{5b}.
(31) Each R^{5a} is absent or methylene.
(32) R^{5b} and R^{5c} are each independently selected from the group consisting of hydrogen, methyl, ethyl and pentyl (e.g. hydrogen, methyl and ethyl).
(33) Each R^{5d} is independently selected from the group consisting of hydroxy, halogen, amino, (1-2C)alkyl, (1-2C)alkoxy and (1-2C)haloalkyl.
(34) Each R^{5d} is independently selected from the group consisting of halogen, (1-2C)alkyl and (1-2C)haloalkyl.
(35) Each W is selected from the group consisting of (1-3C)alkylene, phenylene, 5-6 membered heteroarylene, 5-6 membered carbocyclylene and 5-6 membered heterocyclylene, where any (1-3C)alkylene, phenylene, 5-6 membered heteroarylene, 5-6 membered carbocyclylene or 5-6 membered heterocyclylene in W is optionally substituted with one or more W^{a}.
(36) Each W is selected from the group consisting of (1-3C)alkylene or phenylene, where any (1-3C)alkylene or phenylene in W is optionally substituted with one or more W^{a}.
(37) Each W^{a} is independently selected from the group consisting of hydroxy, halogen, (1-2)alkoxy and (1-2C)haloalkyl.
(38) X is selected from the group consisting of —O—, —C(O)—, —C(O)—O—, —O—C(O)—, —C(O)—N(R^{x})—, —N(R^{x})—C(O)— and —NR^{x}—.
(39) X is selected from the group consisting of —C(O)—N(R^{x})—, —N(R^{x})—C(O)— and —NR^{x}—.
(40) X is —NR^{x}—.
(41) Each R^{x} is independently selected from the group consisting of hydrogen, hydroxy and (1-4C)alkyl.
(42) Each R^{x} is independently selected from the group consisting of hydrogen, hydroxy and methyl.
(43) Each R^{x} is hydrogen.
(44) Each Y is selected from the group consisting of (1-3C)alkylene, phenylene, 5-6 membered heteroarylene, 5-6 membered carbocyclylene and 5-6 membered heterocyclylene, where any (1-3C)alkylene, phenylene, 5-6 membered heteroarylene, 5-6 membered carbocyclylene or 5-6 membered heterocyclylene in W is optionally substituted with one or more Y^{a}.
(45) Each Y is selected from the group consisting of (1-3C)alkylene or phenylene, where any (1-3C)alkylene or phenylene in W is optionally substituted with one or more Y^{a}.
(46) Each Y^{a} is independently selected from the group consisting of hydroxy, halogen, (1-2)alkoxy and (1-2C)haloalkyl.
(47) Z is selected from the group consisting of —O—, —C(O)—, —C(O)—O—, —O—C(O)—, —C(O)—N(R^{z})—, —N(R^{z})—C(O)— and —NR^{z}—.
(48) Z is selected from the group consisting of —C(O)—N(R^{z})—, —N(R^{z})—C(O)— and —NR^{z}—.
(49) Z is —NR^{z}—.
(50) Each R^{z} is independently selected from the group consisting of hydrogen, hydroxy and (1-4C)alkyl.
(51) Each R^{z} is independently selected from the group consisting of hydrogen, hydroxy and methyl.
(52) Each R^{z} is hydrogen.
(53) n is 0 or 1 and o is 0 or 1.
(54) m is 0 and p is 1.
(55) at least one of m, n, o and p is not 0 (i.e. L¹ is not simply a bond).
(56) n is 0 or 1, m is 1, o is 0 or 1 and p is 1.
(57) n is 0 or 1, m is 0, o is 0 or 1 and p is 1.
(58) L¹ has a structure according to any one of the following:

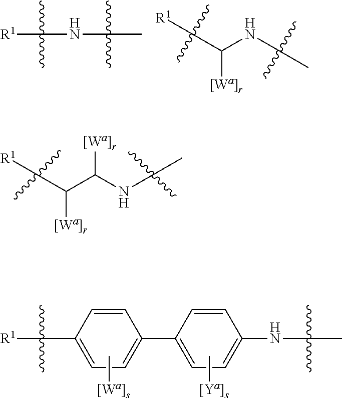

wherein each r is independent 0 or 1; and
each s is independently 0, 1 or 2.
(59) L¹ has a structure according to any one of the following:

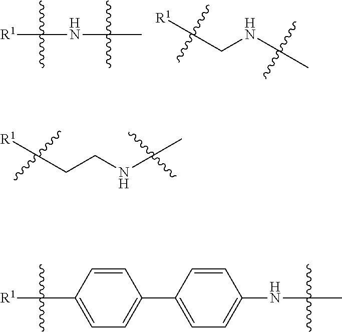

(60) L¹ has a structure according to any one of the following:

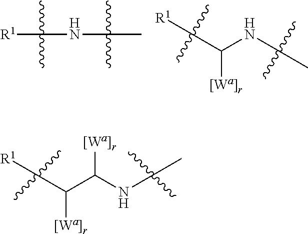

wherein each r is independent 0 or 1
(61) L¹ has a structure according to any one of the following:

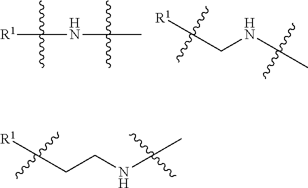

(62) L¹ has a structure according to any one of the following:

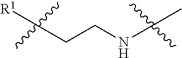

(63) L² is selected from the group consisting of absent (such that B is bonded directly to A) and (1-2C)alkylene, where any (1-2C)alkylene in L² is optionally substituted with one or more substituents selected form the group consisting of hydroxy, halogen, amino, (1-2C)alkyl, (1-2C)alkoxy and (1-2C)haloalkyl.
(64) L² is selected from the group consisting of absent and (1-2C)alkylene, where any (1-2C)alkylene in L² is optionally substituted with one or more substituents selected form the group consisting of hydroxy, halogen and (1-2C)haloalkyl.
(65) L² is selected from the group consisting of absent, methylene and ethylene.
(66) L² is absent (such that ring A is bonded directly to ring B).
(67) R¹ is selected from the group consisting of N, CR², phenyl, 6 membered heteroaryl, 6 membered carbocyclyl and 6 membered heterocyclyl, where any phenyl, 6 membered heteroaryl, 6 membered carbocyclyl or 6 membered heterocyclyl in R¹ is optionally substituted with one or more R³.
(68) R¹ is selected from the group consisting of N, CR², phenyl and cyclohexyl, where any phenyl or cyclohexyl in R¹ is optionally substituted with one or more R³.
(69) R¹ has a structure according to any one of the following:

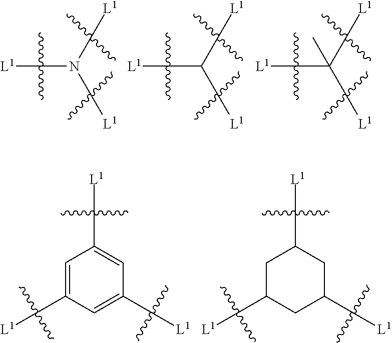

(70) R¹ has a structure according to the following:

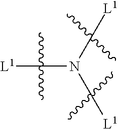

(71) R² is selected from the group consisting of hydrogen, hydroxy, halogen, (1-4C)alkyl, (1-4C)haloalkyl and —OR^{2a}, where any (1-4C)alkyl or (1-4C)haloalkyl in R² is optionally substituted with one or more R^{2c}.
(72) R² is selected from the group consisting of hydrogen, and (1-3C)alkyl, where any (1-4C)alkyl in R² is optionally substituted with one or more R^{2c}.
(73) R² is selected from the group consisting of hydrogen, methyl or ethyl.
(74) Each R^{2a} is independently selected from the group consisting of hydrogen and methyl.
(75) Each R^{2b} is independently selected from the group consisting of hydrogen and methyl.
(76) Each R^{2c} is independently selected from the group consisting of hydroxy, halogen, amino, (1-2C)alkoxy and (1-2C)haloalkyl.
(77) Each R³ is independently selected from the group consisting of hydroxy, halogen, (1-4C)alkyl, (1-4C)haloalkyl and —OR^{3a}, where any (1-4C)alkyl or (1-4C)haloalkyl in R³ is optionally substituted with one or more R^{3C}.
(78) Each R^{3a} is independently selected from the group consisting of hydrogen and methyl.
(79) Each R^{3b} is independently selected from the group consisting of hydrogen and methyl.
(80) Each R^{3c} is independently selected from the group consisting of hydroxy, halogen, amino, (1-2C)alkoxy and (1-2C)haloalkyl.

Suitably, each ring A is as defined in any one of numbered paragraphs (2) to (4). Most suitably, each ring A is as defined in numbered paragraph (4).

Suitably, each X^{a} is as defined in numbered paragraph (6) or (7). Most suitably, each X^{a} is as defined in both of numbered paragraphs (6) and (7).

Suitably, each R⁴ is as defined in any one of numbered paragraphs (9) to (11). Most suitably, each R⁴ is as defined in numbered paragraph (11).

Suitably, R^{4d} is as defined in numbered paragraph (15).

Suitably, each ring B is as defined in any one of numbered paragraphs (17) to (21). More suitably, each ring B is as defined in any one of numbered paragraphs (19) to (21). Most suitably, each ring B is as defined in numbered paragraph (21).

Suitably, each X^{b} is as defined in numbered paragraph (23) or (24). Most suitably, each X^{b} is as defined in both of numbered paragraphs (23) and (24).

Suitably, each R⁵ is as defined in any one of numbered paragraphs (27) to (30). More suitably, each R⁵ is as defined in numbered paragraph (29) or (30). Most suitably, each R⁵ is as defined in numbered paragraph (30).

Suitably, R^{5d} is as defined in numbered paragraph (34).

Suitably, each W is as defined in numbered paragraph (36).

Suitably, X is as defined in numbered paragraph (39) or (40). Most suitably, X is as defined in numbered paragraph (40).

Suitably, each R^{x} is as defined in numbered paragraph (42) or (43). Most suitably, each R^{x} is as defined in numbered paragraph (43).

Suitably, each Y is as defined in numbered paragraph (45).

Suitably, Z is as defined in numbered paragraph (48) or (49). Most suitably, Z is as defined in numbered paragraph (49).

Suitably, each R^{z} is as defined in numbered paragraph (51) or (52). Most suitably, each R^{z} is as defined in numbered paragraph (52).

Suitably, n, m, o and p are as defined in numbered paragraph (55), (56) or (57). Most suitably, n, m, o and p are as defined in numbered paragraph (57).

Suitably, L¹ is as defined in any one of numbered paragraphs (58) to (62). More suitably, L¹ is as defined in any one of numbered paragraphs (60) to (62). Most suitably, L¹ is as defined in numbered paragraph (62).

Suitably, L² is as defined in numbered paragraph (65) or (66). Most suitably, L² is as defined in numbered paragraph (66).

Suitably, R¹ is as defined in numbered paragraph (69) or (70). Most suitably, R¹ is as defined in numbered paragraph (70).

Suitably, R² is as defined in numbered paragraph (72) or (73). Most suitably, R² is as defined in numbered paragraph (73).

In a particular group of compounds of the invention, the compounds have a structure according to Formula I-I shown below (which is a sub-definition of Formula I), or a pharmaceutically-acceptable salt, hydrate and/or solvate thereof:

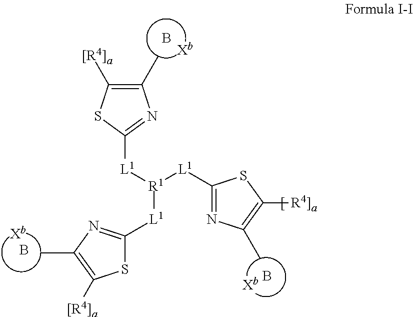

wherein R¹, L¹, R⁴, a, B, X^{b} and any associated subgroups are as defined in any of the numbered paragraphs appearing hereinbefore.

In an embodiment of the compounds of Formula 1-1, or a pharmaceutically-acceptable salt, hydrate and/or solvate thereof, R¹ is as defined in any one of numbered paragraphs (68) to (70).

In an embodiment of the compounds of Formula 1-1, or a pharmaceutically-acceptable salt, hydrate and/or solvate thereof, W is as defined in numbered paragraph (36), X is as defined in numbered paragraph (38), (39) or (40), Y is as defined in numbered paragraph (45) and Z is as defined in numbered paragraph (47), (48) or (49), where m, n, o and p are as defined in numbered paragraph (55).

In an embodiment of the compounds of Formula 1-1, or a pharmaceutically-acceptable salt, hydrate and/or solvate thereof, W is as defined in numbered paragraph (36), X is as defined in numbered paragraph (38), (39) or (40), Y is as defined in numbered paragraph (45) and Z is as defined in numbered paragraph (47), (48) or (49), where m, n, o and p are as defined in numbered paragraph (56) or (57).

In an embodiment of the compounds of Formula 1-1, or a pharmaceutically-acceptable salt, hydrate and/or solvate thereof, L¹ is as defined in any one of numbered paragraphs (58) to (62).

In an embodiment of the compounds of Formula 1-1, or a pharmaceutically-acceptable salt, hydrate and/or solvate thereof, R⁴ is as defined in any one of numbered paragraphs (9) to (11).

In an embodiment of the compounds of Formula 1-1, or a pharmaceutically-acceptable salt, hydrate and/or solvate thereof, each X^{b} is as defined in both of numbered paragraphs (23) and (24).

In an embodiment of the compounds of Formula 1-1, or a pharmaceutically-acceptable salt, hydrate and/or solvate thereof, each ring B is as defined in any one of numbered paragraphs (19) to (21).

In an embodiment of the compounds of Formula 1-1, or a pharmaceutically-acceptable salt, hydrate and/or solvate thereof:

R¹ is as defined in numbered paragraph (69) or (70); and
L¹ is as defined in any one of numbered paragraphs (58) to (62).

In an embodiment of the compounds of Formula 1-1, or a pharmaceutically-acceptable salt, hydrate and/or solvate thereof:

R¹ is as defined in numbered paragraph (69) or (70); and
each ring B is as defined in any one of numbered paragraphs (19) to (21).

In an embodiment of the compounds of Formula 1-1, or a pharmaceutically-acceptable salt, hydrate and/or solvate thereof:

L¹ is as defined in any one of numbered paragraphs (58) to (62); and each ring B is as defined in any one of numbered paragraphs (19) to (21).

In an embodiment of the compounds of Formula 1-1, or a pharmaceutically-acceptable salt, hydrate and/or solvate thereof:

L¹ is as defined in any one of numbered paragraphs (58) to (62); and
each X^{b} is as defined in both of numbered paragraphs (23) and (24).

In an embodiment of the compounds of Formula 1-1, or a pharmaceutically-acceptable salt, hydrate and/or solvate thereof:

R¹ is as defined in numbered paragraph (69) or (70); and
each X^{b} is as defined in both of numbered paragraphs (23) and (24).

In an embodiment of the compounds of Formula 1-1, or a pharmaceutically-acceptable salt, hydrate and/or solvate thereof:

R¹ is as defined in numbered paragraph (69) or (70);
L¹ is as defined in any one of numbered paragraphs (58) to (62); and
each ring B is as defined in any one of numbered paragraphs (19) to (21).

In an embodiment of the compounds of Formula 1-1, or a pharmaceutically-acceptable salt, hydrate and/or solvate thereof:

R¹ is as defined in numbered paragraph (69) or (70);
L¹ is as defined in any one of numbered paragraphs (60) to (62); and
each ring B is as defined in any one of numbered paragraphs (19) to (21).

In an embodiment of the compounds of Formula 1-1, or a pharmaceutically-acceptable salt, hydrate and/or solvate thereof:

R¹ is as defined in numbered paragraph (69) or (70);
L¹ is as defined in any one of numbered paragraphs (60) to (62); and
each ring B is as defined in any one of numbered paragraphs (20) to (21).

In a particular group of compounds of the invention, the compounds have a structure according to Formula I-II shown below (which is a sub-definition of Formula I), or a pharmaceutically-acceptable salt, hydrate and/or solvate thereof:

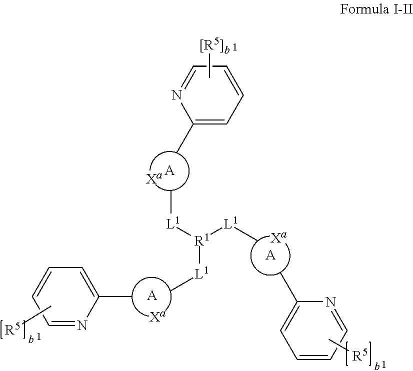

wherein
each R⁵ is as defined in any of the numbered paragraphs appearing hereinbefore, or two R⁵ groups located on adjacent carbon atoms are linked, such that when taken in combination with the atoms to which they are attached, they form a fused benzene ring that is optionally substituted with one or more groups R⁵; and
R¹, L¹, A, X^{a}, b¹ and any associated subgroups are as defined in any of the numbered paragraphs appearing hereinbefore.

In an embodiment of the compounds of Formula I-II, or a pharmaceutically-acceptable salt, hydrate and/or solvate thereof, R¹ is as defined in any one of numbered paragraphs (68) to (70).

In an embodiment of the compounds of Formula I-II, or a pharmaceutically-acceptable salt, hydrate and/or solvate thereof, W is as defined in numbered paragraph (36), X is as defined in numbered paragraph (38), (39) or (40), Y is as defined in numbered paragraph (45) and Z is as defined in numbered paragraph (47), (48) or (49), where m, n, o and p are as defined in numbered paragraph (55).

In an embodiment of the compounds of Formula I-II, or a pharmaceutically-acceptable salt, hydrate and/or solvate thereof, W is as defined in numbered paragraph (36), X is as defined in numbered paragraph (38), (39) or (40), Y is as defined in numbered paragraph (45) and Z is as defined in numbered paragraph (47), (48) or (49), where m, n, o and p are as defined in numbered paragraph (56) or (57).

In an embodiment of the compounds of Formula I-II, or a pharmaceutically-acceptable salt, hydrate and/or solvate thereof, L¹ is as defined in any one of numbered paragraphs (58) to (62).

In an embodiment of the compounds of Formula I-II, or a pharmaceutically-acceptable salt, hydrate and/or solvate thereof, R⁵ is as defined in any one of numbered paragraphs (27) to (30), or two R⁵ groups located on adjacent carbon atoms are linked, such that when taken in combination with the atoms to which they are attached, they form a fused benzene ring that is optionally substituted with one or more groups R⁵ as defined in any one of numbered paragraphs (27) to (30).

In an embodiment of the compounds of Formula I-II, or a pharmaceutically-acceptable salt, hydrate and/or solvate thereof, each X^{a} is as defined in both of numbered paragraphs (6) and (7).

In an embodiment of the compounds of Formula I-II, or a pharmaceutically-acceptable salt, hydrate and/or solvate thereof, each ring A is as defined in any one of numbered paragraphs (2) to (4).

In an embodiment of the compounds of Formula I-II, or a pharmaceutically-acceptable salt, hydrate and/or solvate thereof:

R¹ is as defined in numbered paragraph (69) or (70); and
L¹ is as defined in any one of numbered paragraphs (58) to (62).

In an embodiment of the compounds of Formula I-II, or a pharmaceutically-acceptable salt, hydrate and/or solvate thereof:

R¹ is as defined in numbered paragraph (69) or (70); and
each ring A is as defined in any one of numbered paragraphs (2) to (4).

In an embodiment of the compounds of Formula I-II, or a pharmaceutically-acceptable salt, hydrate and/or solvate thereof:

L¹ is as defined in any one of numbered paragraphs (58) to (62); and
each ring A is as defined in any one of numbered paragraphs (2) to (4).

In an embodiment of the compounds of Formula I-II, or a pharmaceutically-acceptable salt, hydrate and/or solvate thereof:

L¹ is as defined in any one of numbered paragraphs (58) to (62); and
each X^{a} is as defined in both of numbered paragraphs (6) and (7).

In an embodiment of the compounds of Formula I-II, or a pharmaceutically-acceptable salt, hydrate and/or solvate thereof:

R¹ is as defined in numbered paragraph (69) or (70); and
each X^{a} is as defined in both of numbered paragraphs (6) and (7).

In an embodiment of the compounds of Formula I-II, or a pharmaceutically-acceptable salt, hydrate and/or solvate thereof:

R¹ is as defined in numbered paragraph (69) or (70);
L¹ is as defined in any one of numbered paragraphs (58) to (62); and
each ring A is as defined in any one of numbered paragraphs (2) to (4).

In an embodiment of the compounds of Formula I-II, or a pharmaceutically-acceptable salt, hydrate and/or solvate thereof:

R¹ is as defined in numbered paragraph (69) or (70);
L¹ is as defined in any one of numbered paragraphs (60) to (62); and
each ring A is as defined in any one of numbered paragraphs (2) to (4).

In an embodiment of the compounds of Formula I-II, or a pharmaceutically-acceptable salt, hydrate and/or solvate thereof:

R¹ is as defined in numbered paragraph (69) or (70);
L¹ is as defined in any one of numbered paragraphs (60) to (62); and
each ring A is as defined in any one of numbered paragraphs (3) to (4).

In a particular group of compounds of the invention, the compounds have a structure according to Formula I-III shown below (which is a sub-definition of Formula I), or a pharmaceutically-acceptable salt, hydrate and/or solvate thereof:

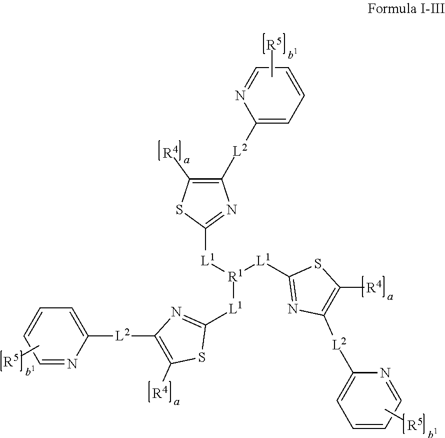

wherein
each R⁵ is as defined in any of the numbered paragraphs appearing hereinbefore, or two R⁵ groups located on adjacent carbon atoms are linked, such that when taken in combination with the atoms to which they are attached, they form a fused benzene ring that is optionally substituted with one or more groups R⁵ as defined in any of the numbered paragraphs appearing hereinbefore; and
R¹, L¹, R⁴, a, L², b¹ and any associated subgroups are as defined in any of the numbered paragraphs appearing hereinbefore.

In an embodiment of the compounds of Formula I-III, or a pharmaceutically-acceptable salt, hydrate and/or solvate thereof, R¹ is as defined in any one of numbered paragraphs (68) to (70).

In an embodiment of the compounds of Formula I-III, or a pharmaceutically-acceptable salt, hydrate and/or solvate thereof, W is as defined in numbered paragraph (36), X is as defined in numbered paragraph (38), (39) or (40), Y is as defined in numbered paragraph (45) and Z is as defined in numbered paragraph (47), (48) or (49), where m, n, o and p are as defined in numbered paragraph (55).

In an embodiment of the compounds of Formula I-III, or a pharmaceutically-acceptable salt, hydrate and/or solvate thereof, W is as defined in numbered paragraph (36), X is as defined in numbered paragraph (38), (39) or (40), Y is as defined in numbered paragraph (45) and Z is as defined in numbered paragraph (47), (48) or (49), where m, n, o and p are as defined in numbered paragraph (56) or (57).

In an embodiment of the compounds of Formula I-III, or a pharmaceutically-acceptable salt, hydrate and/or solvate thereof, L¹ is as defined in any one of numbered paragraphs (58) to (62).

In an embodiment of the compounds of Formula I-III, or a pharmaceutically-acceptable salt, hydrate and/or solvate thereof, R⁴ is as defined in any one of numbered paragraphs (9) to (11).

In an embodiment of the compounds of Formula I-III, or a pharmaceutically-acceptable salt, hydrate and/or solvate thereof, R⁵ is as defined in any one of numbered paragraphs (27) to (30), or two R⁵ groups located on adjacent carbon atoms are linked, such that when taken in combination with the atoms to which they are attached, they form a fused benzene ring that is optionally substituted with one or more groups R⁵ as defined in any one of numbered paragraphs (27) to (30).

In an embodiment of the compounds of Formula I-III, or a pharmaceutically-acceptable salt, hydrate and/or solvate thereof, each L² is as defined in numbered paragraph (65) or (66).

In an embodiment of the compounds of Formula I-III, or a pharmaceutically-acceptable salt, hydrate and/or solvate thereof:

R¹ is as defined in numbered paragraph (69) or (70); and
L¹ is as defined in any one of numbered paragraphs (58) to (62).

In an embodiment of the compounds of Formula I-III, or a pharmaceutically-acceptable salt, hydrate and/or solvate thereof:

R¹ is as defined in numbered paragraph (69) or (70); and
L² is as defined in numbered paragraph (65) or (66).

In an embodiment of the compounds of Formula I-III, or a pharmaceutically-acceptable salt, hydrate and/or solvate thereof:

L² is as defined in numbered paragraph (65) or (66); and
L¹ is as defined in any one of numbered paragraphs (58) to (62).

In an embodiment of the compounds of Formula I-III, or a pharmaceutically-acceptable salt, hydrate and/or solvate thereof:

R¹ is as defined in numbered paragraph (69) or (70);
L¹ is as defined in any one of numbered paragraphs (58) to (62); and
L² is as defined in numbered paragraph (65) or (66).

In an embodiment of the compounds of Formula I-III, or a pharmaceutically-acceptable salt, hydrate and/or solvate thereof:

R¹ is as defined in numbered paragraph (69) or (70);
L¹ is as defined in any one of numbered paragraphs (60) to (62); and
L² is as defined in numbered paragraph (65) or (66).

In an embodiment of the compounds of Formula I-III, or a pharmaceutically-acceptable salt, hydrate and/or solvate thereof:

R¹ is as defined in numbered paragraph (69) or (70);
L¹ is as defined in any one of numbered paragraphs (60) to (62); and
L² is as defined in numbered paragraph (66).

In a particular group of compounds of the invention, the compounds have a structure according to Formula I-IV shown below (which is a sub-definition of Formula I), or a pharmaceutically-acceptable salt, hydrate and/or solvate thereof:

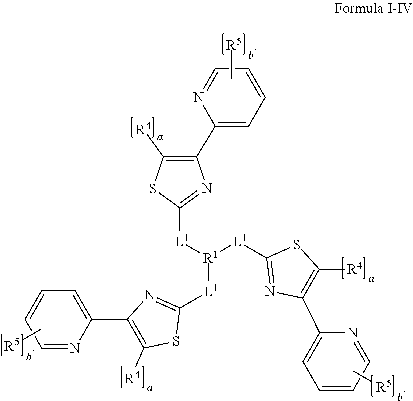

wherein
each R⁵ is as defined in any of the numbered paragraphs appearing hereinbefore, or two R⁵ groups located on adjacent carbon atoms are linked, such that when taken in combination with the atoms to which they are attached, they form a fused benzene ring that is optionally substituted with one or more groups R⁵ as defined in any of the numbered paragraphs appearing hereinbefore; and
R¹, L¹, R⁴, a, b¹ and any associated subgroups are as defined in any of the numbered paragraphs appearing hereinbefore.

In an embodiment of the compounds of Formula I-IV, or a pharmaceutically-acceptable salt, hydrate and/or solvate thereof, R¹ is as defined in any one of numbered paragraphs (68) to (70).

In an embodiment of the compounds of Formula I-IV, or a pharmaceutically-acceptable salt, hydrate and/or solvate thereof, W is as defined in numbered paragraph (36), X is as defined in numbered paragraph (38), (39) or (40), Y is as defined in numbered paragraph (45) and Z is as defined in numbered paragraph (47), (48) or (49), where m, n, o and p are as defined in numbered paragraph (55).

In an embodiment of the compounds of Formula I-IV, or a pharmaceutically-acceptable salt, hydrate and/or solvate thereof, W is as defined in numbered paragraph (36), X is as defined in numbered paragraph (38), (39) or (40), Y is as defined in numbered paragraph (45) and Z is as defined in numbered paragraph (47), (48) or (49), where m, n, o and p are as defined in numbered paragraph (56) or (57).

In an embodiment of the compounds of Formula I-IV, or a pharmaceutically-acceptable salt, hydrate and/or solvate thereof, L¹ is as defined in any one of numbered paragraphs (58) to (62).

In an embodiment of the compounds of Formula I-IV, or a pharmaceutically-acceptable salt, hydrate and/or solvate thereof, R⁴ is as defined in any one of numbered paragraphs (9) to (11).

In an embodiment of the compounds of Formula I-IV, or a pharmaceutically-acceptable salt, hydrate and/or solvate thereof, R⁵ is as defined in any one of numbered paragraphs (27) to (30), or two R⁵ groups located on adjacent carbon atoms are linked, such that when taken in combination with the atoms to which they are attached, they form a fused benzene ring that is optionally substituted with one or more groups R⁵ as defined in any one of numbered paragraphs (27) to (30).

In an embodiment of the compounds of Formula I-IV, or a pharmaceutically-acceptable salt, hydrate and/or solvate thereof:

R¹ is as defined in any one of numbered paragraphs (68) to (70); and
L¹ is as defined in any one of numbered paragraphs (58) to (62).

In an embodiment of the compounds of Formula I-IV, or a pharmaceutically-acceptable salt, hydrate and/or solvate thereof:

R¹ is as defined in numbered paragraph (69) or (70); and
L¹ is as defined in any one of numbered paragraphs (60) to (62).

In a particular group of compounds of the invention, the compounds have a structure according to Formula I-V shown below (which is a sub-definition of Formula I), or a pharmaceutically-acceptable salt, hydrate and/or solvate thereof:

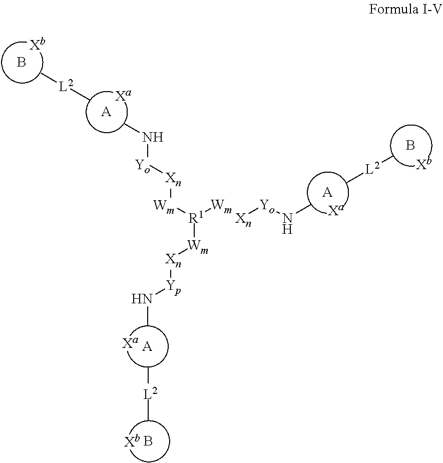

wherein R¹, W, X, Y, m, n, o, A, X^{a}, L², B, X^{b} and any associated subgroups are as defined in any of the numbered paragraphs appearing hereinbefore.

In an embodiment of the compounds of Formula I-V, or a pharmaceutically-acceptable salt, hydrate and/or solvate thereof, R¹ is as defined in any one of numbered paragraphs (68) to (70).

In an embodiment of the compounds of Formula I-V, or a pharmaceutically-acceptable salt, hydrate and/or solvate thereof, W is as defined in numbered paragraph (36), X is as defined in numbered paragraph (38), (39) or (40) and Y is as defined in numbered paragraph (45).

In an embodiment of the compounds of Formula I-V, or a pharmaceutically-acceptable salt, hydrate and/or solvate thereof, W is as defined in numbered paragraph (36), X is as defined in numbered paragraph (38), (39) or (40), Y is as defined in numbered paragraph (45) and m, n and o are as defined in numbered paragraph (56) or (57).

In an embodiment of the compounds of Formula I-V, or a pharmaceutically-acceptable salt, hydrate and/or solvate thereof, each X^{a} is as defined in both of numbered paragraphs (6) and (7).

In an embodiment of the compounds of Formula I-V, or a pharmaceutically-acceptable salt, hydrate and/or solvate thereof, each ring A is as defined in any one of numbered paragraphs (2) to (4).

In an embodiment of the compounds of Formula I-V, or a pharmaceutically-acceptable salt, hydrate and/or solvate thereof, each X^{b} is as defined in both of numbered paragraphs (23) and (24).

In an embodiment of the compounds of Formula I-V, or a pharmaceutically-acceptable salt, hydrate and/or solvate thereof, each ring B is as defined in any one of numbered paragraphs (19) to (21).

In an embodiment of the compounds of Formula I-V, or a pharmaceutically-acceptable salt, hydrate and/or solvate thereof, each L² is as defined in numbered paragraph (65) or (66).

In an embodiment of the compounds of Formula I-V, or a pharmaceutically-acceptable salt, hydrate and/or solvate thereof:

R¹ is as defined in numbered paragraph (69) or (70); and
each ring B is as defined in any one of numbered paragraphs (19) to (21).

In an embodiment of the compounds of Formula I-V, or a pharmaceutically-acceptable salt, hydrate and/or solvate thereof:

R¹ is as defined in numbered paragraph (69) or (70); and
each X^{b} is as defined in both of numbered paragraphs (23) and (24).

In an embodiment of the compounds of Formula I-V, or a pharmaceutically-acceptable salt, hydrate and/or solvate thereof:

R¹ is as defined in numbered paragraph (69) or (70); and
each ring A is as defined in any one of numbered paragraphs (2) to (4).

In an embodiment of the compounds of Formula I-V, or a pharmaceutically-acceptable salt, hydrate and/or solvate thereof:

R¹ is as defined in numbered paragraph (69) or (70); and
each X^{a} is as defined in both of numbered paragraphs (6) and (7).

In an embodiment of the compounds of Formula I-V, or a pharmaceutically-acceptable salt, hydrate and/or solvate thereof:

R¹ is as defined in numbered paragraph (69) or (70); and
W is as defined in numbered paragraph (36), X is as defined in numbered paragraph (38), (39) or (40) and Y is as defined in numbered paragraph (45).

In an embodiment of the compounds of Formula I-V, or a pharmaceutically-acceptable salt, hydrate and/or solvate thereof:

each X^{a} is as defined in both of numbered paragraphs (6) and (7);
each X^{b} is as defined in both of numbered paragraphs (23) and (24); and
W is as defined in numbered paragraph (36), X is as defined in numbered paragraph (38), (39) or (40) and Y is as defined in numbered paragraph (45).

In an embodiment of the compounds of Formula I-V, or a pharmaceutically-acceptable salt, hydrate and/or solvate thereof:

each ring A is as defined in any one of numbered paragraphs (2) to (4);
each ring B is as defined in any one of numbered paragraphs (19) to (21); and
W is as defined in numbered paragraph (36), X is as defined in numbered paragraph (38), (39) or (40) and Y is as defined in numbered paragraph (45).

In an embodiment of the compounds of Formula I-V, or a pharmaceutically-acceptable salt, hydrate and/or solvate thereof:

R¹ is as defined in numbered paragraph (69) or (70);
each X^{a} is as defined in both of numbered paragraphs (6) and (7);
each X^{b} is as defined in both of numbered paragraphs (23) and (24); and
W is as defined in numbered paragraph (36), X is as defined in numbered paragraph (38), (39) or (40) and Y is as defined in numbered paragraph (45).

In an embodiment of the compounds of Formula I-V, or a pharmaceutically-acceptable salt, hydrate and/or solvate thereof:

R¹ is as defined in numbered paragraph (69) or (70);
each ring A is as defined in any one of numbered paragraphs (2) to (4);
each ring B is as defined in any one of numbered paragraphs (19) to (21); and
W is as defined in numbered paragraph (36), X is as defined in numbered paragraph (38), (39) or (40) and Y is as defined in numbered paragraph (45).

In an embodiment of the compounds of Formula I-V, or a pharmaceutically-acceptable salt, hydrate and/or solvate thereof:

L² is as defined in numbered paragraph (65) or (66);
each X^{a} is as defined in both of numbered paragraphs (6) and (7);
each X^{b} is as defined in both of numbered paragraphs (23) and (24); and
W is as defined in numbered paragraph (36), X is as defined in numbered paragraph (38), (39) or (40) and Y is as defined in numbered paragraph (45).

In an embodiment of the compounds of Formula I-V, or a pharmaceutically-acceptable salt, hydrate and/or solvate thereof:

L² is as defined in numbered paragraph (65) or (66);
each ring A is as defined in any one of numbered paragraphs (2) to (4);
each ring B is as defined in any one of numbered paragraphs (19) to (21); and
W is as defined in numbered paragraph (36), X is as defined in numbered paragraph (38), (39) or (40) and Y is as defined in numbered paragraph (45).

In an embodiment of the compounds of Formula I-V, or a pharmaceutically-acceptable salt, hydrate and/or solvate thereof:

R¹ is as defined in numbered paragraph (69) or (70);
L² is as defined in numbered paragraph (65) or (66);
each X^{a} is as defined in both of numbered paragraphs (6) and (7);
each X^{b} is as defined in both of numbered paragraphs (23) and (24); and
W is as defined in numbered paragraph (36), X is as defined in numbered paragraph (38), (39) or (40) and Y is as defined in numbered paragraph (45).

In an embodiment of the compounds of Formula I-V, or a pharmaceutically-acceptable salt, hydrate and/or solvate thereof:

R¹ is as defined in numbered paragraph (69) or (70);
L² is as defined in numbered paragraph (65) or (66);
each ring A is as defined in any one of numbered paragraphs (2) to (4);
each ring B is as defined in any one of numbered paragraphs (19) to (21); and
W is as defined in numbered paragraph (36), X is as defined in numbered paragraph (38), (39) or (40) and Y is as defined in numbered paragraph (45).

In a particular group of compounds of the invention, the compounds have a structure according to Formula I-VI shown below (which is a sub-definition of Formula I), or a pharmaceutically-acceptable salt, hydrate and/or solvate thereof:

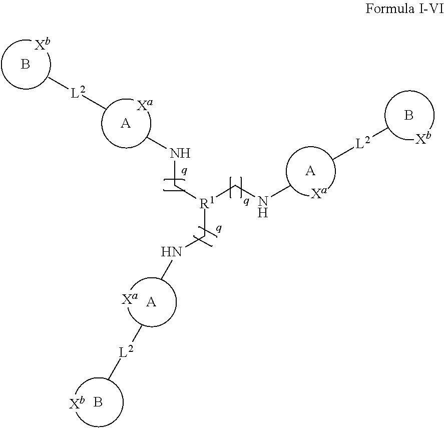

wherein q is 0, 1, 2 or 3; and
R¹, A, X^{a}, L², B, X^{b} and any associated subgroups are as defined in any of the numbered paragraphs appearing hereinbefore.

In an embodiment of the compounds of Formula I-VI, or a pharmaceutically-acceptable salt, hydrate and/or solvate thereof, q is 0, 1 or 2.

In an embodiment of the compounds of Formula I-VI, or a pharmaceutically-acceptable salt, hydrate and/or solvate thereof, R¹ is as defined in any one of numbered paragraphs (68) to (70).

In an embodiment of the compounds of Formula I-VI, or a pharmaceutically-acceptable salt, hydrate and/or solvate thereof, each X^{a} is as defined in both of numbered paragraphs (6) and (7).

In an embodiment of the compounds of Formula I-VI, or a pharmaceutically-acceptable salt, hydrate and/or solvate thereof, each ring A is as defined in any one of numbered paragraphs (2) to (4).

In an embodiment of the compounds of Formula I-VI, or a pharmaceutically-acceptable salt, hydrate and/or solvate thereof, each X^{b} is as defined in both of numbered paragraphs (23) and (24).

In an embodiment of the compounds of Formula I-VI, or a pharmaceutically-acceptable salt, hydrate and/or solvate thereof, each ring B is as defined in any one of numbered paragraphs (19) to (21).

In an embodiment of the compounds of Formula I-VI, or a pharmaceutically-acceptable salt, hydrate and/or solvate thereof, each L² is as defined in numbered paragraph (65) or (66).

In an embodiment of the compounds of Formula I-VI, or a pharmaceutically-acceptable salt, hydrate and/or solvate thereof:

R¹ is as defined in numbered paragraph (69) or (70); and
each ring B is as defined in any one of numbered paragraphs (19) to (21).

In an embodiment of the compounds of Formula I-VI, or a pharmaceutically-acceptable salt, hydrate and/or solvate thereof:

R¹ is as defined in numbered paragraph (69) or (70); and
each X^{b} is as defined in both of numbered paragraphs (23) and (24).

In an embodiment of the compounds of Formula I-VI, or a pharmaceutically-acceptable salt, hydrate and/or solvate thereof:

R¹ is as defined in numbered paragraph (69) or (70); and
each ring A is as defined in any one of numbered paragraphs (2) to (4).

In an embodiment of the compounds of Formula I-VI, or a pharmaceutically-acceptable salt, hydrate and/or solvate thereof:

R¹ is as defined in numbered paragraph (69) or (70); and
each X^{a} is as defined in both of numbered paragraphs (6) and (7).

In an embodiment of the compounds of Formula I-VI, or a pharmaceutically-acceptable salt, hydrate and/or solvate thereof:

R¹ is as defined in numbered paragraph (69) or (70);
each X^{a} is as defined in both of numbered paragraphs (6) and (7); and
each X^{b} is as defined in both of numbered paragraphs (23) and (24).

In an embodiment of the compounds of Formula I-VI, or a pharmaceutically-acceptable salt, hydrate and/or solvate thereof:

R¹ is as defined in numbered paragraph (69) or (70);
each ring A is as defined in any one of numbered paragraphs (2) to (4); and
each ring B is as defined in any one of numbered paragraphs (19) to (21).

In an embodiment of the compounds of Formula I-VI, or a pharmaceutically-acceptable salt, hydrate and/or solvate thereof:

L² is as defined in numbered paragraph (65) or (66);
each X^{a} is as defined in both of numbered paragraphs (6) and (7); and
each X^{b} is as defined in both of numbered paragraphs (23) and (24).

In an embodiment of the compounds of Formula I-VI, or a pharmaceutically-acceptable salt, hydrate and/or solvate thereof:

L² is as defined in numbered paragraph (65) or (66);
each ring A is as defined in any one of numbered paragraphs (2) to (4); and
each ring B is as defined in any one of numbered paragraphs (19) to (21).

In an embodiment of the compounds of Formula I-VI, or a pharmaceutically-acceptable salt, hydrate and/or solvate thereof:

R¹ is as defined in numbered paragraph (69) or (70);
L² is as defined in numbered paragraph (65) or (66);
each X^{a} is as defined in both of numbered paragraphs (6) and (7); and
each X^{b} is as defined in both of numbered paragraphs (23) and (24).

In an embodiment of the compounds of Formula I-VI, or a pharmaceutically-acceptable salt, hydrate and/or solvate thereof:

R¹ is as defined in numbered paragraph (69) or (70);
L² is as defined in numbered paragraph (65) or (66);
each ring A is as defined in any one of numbered paragraphs (2) to (4); and
each ring B is as defined in any one of numbered paragraphs (19) to (21).

In a particular group of compounds of the invention, the compounds have a structure according to Formula I-VII shown below (which is a sub-definition of Formula I), or a pharmaceutically-acceptable salt, hydrate and/or solvate thereof:

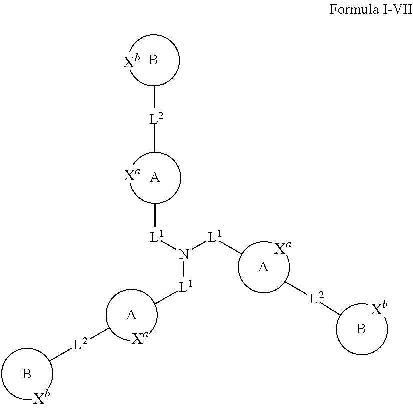

wherein L¹, A, X^{a}, L², B, X^{b} and any associated subgroups are as defined in any of the numbered paragraphs appearing hereinbefore.

In an embodiment of the compounds of Formula I-VII, or a pharmaceutically-acceptable salt, hydrate and/or solvate thereof, W is as defined in numbered paragraph (36), X is as defined in numbered paragraph (38), (39) or (40), Y is as defined in numbered paragraph (45) and Z is as defined in numbered paragraph (47), (48) or (49), where m, n, o and p are as defined in numbered paragraph (55).

In an embodiment of the compounds of Formula I-VII, or a pharmaceutically-acceptable salt, hydrate and/or solvate thereof, W is as defined in numbered paragraph (36), X is as defined in numbered paragraph (38), (39) or (40), Y is as defined in numbered paragraph (45) and Z is as defined in numbered paragraph (47), (48) or (49), where m, n, o and p are as defined in numbered paragraph (56) or (57).

In an embodiment of the compounds of Formula I-VII, or a pharmaceutically-acceptable salt, hydrate and/or solvate thereof, L¹ is as defined in any one of numbered paragraphs (58) to (62).

In an embodiment of the compounds of Formula I-VII, or a pharmaceutically-acceptable salt, hydrate and/or solvate thereof, each X^{a} is as defined in both of numbered paragraphs (6) and (7).

In an embodiment of the compounds of Formula I-VII, or a pharmaceutically-acceptable salt, hydrate and/or solvate thereof, each ring A is as defined in any one of numbered paragraphs (2) to (4).

In an embodiment of the compounds of Formula I-VII, or a pharmaceutically-acceptable salt, hydrate and/or solvate thereof, each X^{b} is as defined in both of numbered paragraphs (23) and (24).

In an embodiment of the compounds of Formula I-VII, or a pharmaceutically-acceptable salt, hydrate and/or solvate thereof, each ring B is as defined in any one of numbered paragraphs (19) to (21).

In an embodiment of the compounds of Formula I-VII, or a pharmaceutically-acceptable salt, hydrate and/or solvate thereof, each L² is as defined in numbered paragraph (65) or (66).

In an embodiment of the compounds of Formula I-VII, or a pharmaceutically-acceptable salt, hydrate and/or solvate thereof:

L¹ is as defined in any one of numbered paragraphs (58) to (62); and
each ring B is as defined in any one of numbered paragraphs (19) to (21).

In an embodiment of the compounds of Formula I-VII, or a pharmaceutically-acceptable salt, hydrate and/or solvate thereof:

L¹ is as defined in any one of numbered paragraphs (58) to (62); and
each X^{b} is as defined in both of numbered paragraphs (23) and (24).

In an embodiment of the compounds of Formula I-VII, or a pharmaceutically-acceptable salt, hydrate and/or solvate thereof:

L¹ is as defined in any one of numbered paragraphs (58) to (62); and
each ring A is as defined in any one of numbered paragraphs (2) to (4).

In an embodiment of the compounds of Formula I-VII, or a pharmaceutically-acceptable salt, hydrate and/or solvate thereof:

L¹ is as defined in any one of numbered paragraphs (58) to (62); and
each X^{a} is as defined in both of numbered paragraphs (6) and (7).

In an embodiment of the compounds of Formula I-VII, or a pharmaceutically-acceptable salt, hydrate and/or solvate thereof:

L¹ is as defined in any one of numbered paragraphs (58) to (62);
each X^{a} is as defined in both of numbered paragraphs (6) and (7); and
each X^{b} is as defined in both of numbered paragraphs (23) and (24).

In an embodiment of the compounds of Formula I-VII, or a pharmaceutically-acceptable salt, hydrate and/or solvate thereof:

L¹ is as defined in any one of numbered paragraphs (58) to (62);
each ring A is as defined in any one of numbered paragraphs (2) to (4); and
each ring B is as defined in any one of numbered paragraphs (19) to (21).

In an embodiment of the compounds of Formula I-VII, or a pharmaceutically-acceptable salt, hydrate and/or solvate thereof:

L² is as defined in numbered paragraph (65) or (66);
each X^{a} is as defined in both of numbered paragraphs (6) and (7); and
each X^{b} is as defined in both of numbered paragraphs (23) and (24).

In an embodiment of the compounds of Formula I-VII, or a pharmaceutically-acceptable salt, hydrate and/or solvate thereof:

L² is as defined in numbered paragraph (65) or (66);
each ring A is as defined in any one of numbered paragraphs (2) to (4); and
each ring B is as defined in any one of numbered paragraphs (19) to (21).

In an embodiment of the compounds of Formula I-VII, or a pharmaceutically-acceptable salt, hydrate and/or solvate thereof:

L¹ is as defined in any one of numbered paragraphs (60) to (62);
L² is as defined in numbered paragraph (65) or (66);
each X^{a} is as defined in both of numbered paragraphs (6) and (7); and
each X^{b} is as defined in both of numbered paragraphs (23) and (24).

In an embodiment of the compounds of Formula I-VII, or a pharmaceutically-acceptable salt, hydrate and/or solvate thereof:

L¹ is as defined in any one of numbered paragraphs (60) to (62);
L² is as defined in numbered paragraph (65) or (66);
each ring A is as defined in any one of numbered paragraphs (2) to (4); and
each ring B is as defined in any one of numbered paragraphs (19) to (21).

In a particular group of compounds of the invention, the compounds have a structure according to Formula I-VIII shown below (which is a sub-definition of Formula I), or a pharmaceutically-acceptable salt, hydrate and/or solvate thereof:

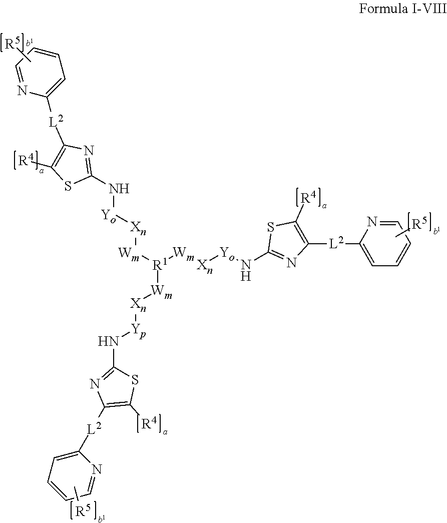

wherein each R⁵ is as defined in any of the numbered paragraphs appearing hereinbefore, or two R⁵ groups located on adjacent carbon atoms are linked, such that when taken in combination with the atoms to which they are attached, they form a fused benzene ring that is optionally substituted with one or more groups R⁵ as defined in any of the numbered paragraphs appearing hereinbefore; and
R¹, W, X, Y, m, n, o, R⁴, a, L², b¹ and any associated subgroups are as defined in any of the numbered paragraphs appearing hereinbefore.

In an embodiment of the compounds of Formula I-VIII, or a pharmaceutically-acceptable salt, hydrate and/or solvate thereof, R¹ is as defined in any one of numbered paragraphs (68) to (70).

In an embodiment of the compounds of Formula I-VIII, or a pharmaceutically-acceptable salt, hydrate and/or solvate thereof, W is as defined in numbered paragraph (36), X is as defined in numbered paragraph (38), (39) or (40) and Y is as defined in numbered paragraph (45).

In an embodiment of the compounds of Formula I-VIII, or a pharmaceutically-acceptable salt, hydrate and/or solvate thereof, W is as defined in numbered paragraph (36), X is as defined in numbered paragraph (38), (39) or (40), Y is as defined in numbered paragraph (45) and m, n and o are as defined in numbered paragraph (56) or (57).

In an embodiment of the compounds of Formula I-VIII, or a pharmaceutically-acceptable salt, hydrate and/or solvate thereof, R⁴ is as defined in any one of numbered paragraphs (9) to (11).

In an embodiment of the compounds of Formula I-VIII, or a pharmaceutically-acceptable salt, hydrate and/or solvate thereof, R⁵ is as defined in any one of numbered paragraphs (27) to (30), or two R⁵ groups located on adjacent carbon atoms are linked, such that when taken in combination with the atoms to which they are attached, they form a fused benzene ring that is optionally substituted with one or more groups R⁵ as defined in any one of numbered paragraphs (27) to (30).

In an embodiment of the compounds of Formula I-VIII, or a pharmaceutically-acceptable salt, hydrate and/or solvate thereof, each L² is as defined in numbered paragraph (65) or (66).

In an embodiment of the compounds of Formula I-VIII, or a pharmaceutically-acceptable salt, hydrate and/or solvate thereof:

R¹ is as defined in numbered paragraph (69) or (70); and
L² is as defined in numbered paragraph (65) or (66).

In an embodiment of the compounds of Formula I-VIII, or a pharmaceutically-acceptable salt, hydrate and/or solvate thereof:

R¹ is as defined in numbered paragraph (69) or (70); and
W is as defined in numbered paragraph (36), X is as defined in numbered paragraph (38), (39) or (40) and Y is as defined in numbered paragraph (45).

In an embodiment of the compounds of Formula I-VIII, or a pharmaceutically-acceptable salt, hydrate and/or solvate thereof:

L² is as defined in numbered paragraph (65) or (66); and
W is as defined in numbered paragraph (36), X is as defined in numbered paragraph (38), (39) or (40) and Y is as defined in numbered paragraph (45).

In an embodiment of the compounds of Formula I-VIII, or a pharmaceutically-acceptable salt, hydrate and/or solvate thereof:

R¹ is as defined in numbered paragraph (69) or (70);
L² is as defined in numbered paragraph (65) or (66); and
W is as defined in numbered paragraph (36), X is as defined in numbered paragraph (38), (39) or (40) and Y is as defined in numbered paragraph (45).

In a particular group of compounds of the invention, the compounds have a structure according to Formula I-IX shown below (which is a sub-definition of Formula I), or a pharmaceutically-acceptable salt, hydrate and/or solvate thereof:

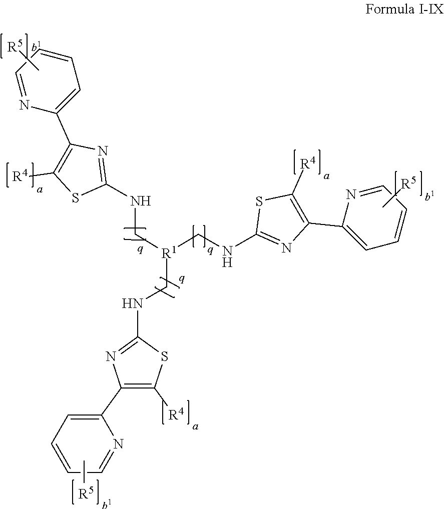

wherein
q is 0 (in which case R¹ is bonded directly to N), 1, 2 or 3;
each R⁵ is as defined in any of the numbered paragraphs appearing hereinbefore, or two R⁵ groups located on adjacent carbon atoms are linked, such that when taken in combination with the atoms to which they are attached, they form a fused benzene ring that is optionally substituted with one or more groups R⁵ as defined in any of the numbered paragraphs appearing hereinbefore; and
R¹, R⁴, a, b¹ and any associated subgroups are as defined in any of the numbered paragraphs appearing hereinbefore.

In an embodiment of the compounds of Formula I-IX, or a pharmaceutically-acceptable salt, hydrate and/or solvate thereof, q is 0, 1 or 2.

In an embodiment of the compounds of Formula I-IX, or a pharmaceutically-acceptable salt, hydrate and/or solvate thereof, R¹ is as defined in any one of numbered paragraphs (68) to (70).

In an embodiment of the compounds of Formula I-IX, or a pharmaceutically-acceptable salt, hydrate and/or solvate thereof, R⁴ is as defined in any one of numbered paragraphs (9) to (11).

In an embodiment of the compounds of Formula I-IX, or a pharmaceutically-acceptable salt, hydrate and/or solvate thereof, R⁵ is as defined in any one of numbered paragraphs (27) to (30), or two R⁵ groups located on adjacent carbon atoms are linked, such that when taken in combination with the atoms to which they are attached, they form a fused benzene ring that is optionally substituted with one or more groups R⁵ as defined in any one of numbered paragraphs (27) to (30).

In an embodiment of the compounds of Formula I-IX, or a pharmaceutically-acceptable salt, hydrate and/or solvate thereof:

R¹ is as defined in numbered paragraph (69) or (70); and
R⁴ is as defined in any one of numbered paragraphs (9) to (11).

In an embodiment of the compounds of Formula I-IX, or a pharmaceutically-acceptable salt, hydrate and/or solvate thereof:

R¹ is as defined in numbered paragraph (69) or (70); and
R⁵ is as defined in any one of numbered paragraphs (27) to (30), or two R⁵ groups located on adjacent carbon atoms are linked, such that when taken in combination with the atoms to which they are attached, they form a fused benzene ring that is optionally substituted with one or more groups R⁵ as defined in any one of numbered paragraphs (27) to (30).

In an embodiment of the compounds of Formula I-IX, or a pharmaceutically-acceptable salt, hydrate and/or solvate thereof:

R⁴ is as defined in any one of numbered paragraphs (9) to (11); and
R⁵ is as defined in any one of numbered paragraphs (27) to (30), or two R⁵ groups located on adjacent carbon atoms are linked, such that when taken in combination with the atoms to which they are attached, they form a fused benzene ring that is optionally substituted with one or more groups R⁵ as defined in any one of numbered paragraphs (27) to (30).

In an embodiment of the compounds of Formula I-IX, or a pharmaceutically-acceptable salt, hydrate and/or solvate thereof:

R¹ is as defined in numbered paragraph (69) or (70);
R⁴ is as defined in any one of numbered paragraphs (9) to (11); and
R⁵ is as defined in any one of numbered paragraphs (27) to (30), or two R⁵ groups located on adjacent carbon atoms are linked, such that when taken in combination with the atoms to which they are attached, they form a fused benzene ring that is optionally substituted with one or more groups R⁵ as defined in any one of numbered paragraphs (27) to (30).

In a particular group of compounds of the invention, the compounds have a structure according to Formula I-X shown below (which is a sub-definition of Formula I), or a pharmaceutically-acceptable salt, hydrate and/or solvate thereof:

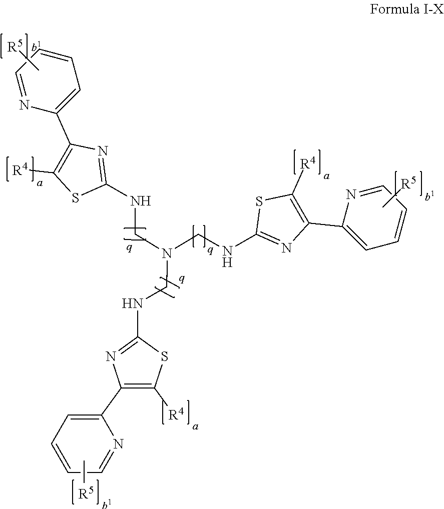

wherein
q is 0 (in which case R¹ is bonded directly to N), 1, 2 or 3;
each R⁵ is as defined in any of the numbered paragraphs appearing hereinbefore, or two R⁵ groups located on adjacent carbon atoms are linked, such that when taken in combination with the atoms to which they are attached, they form a fused benzene ring that is optionally substituted with one or more groups R⁵ as defined in any of the numbered paragraphs appearing hereinbefore; and
R⁴, a, b¹ and any associated subgroups are as defined in any of the numbered paragraphs appearing hereinbefore.

In an embodiment of the compounds of Formula I-X, or a pharmaceutically-acceptable salt, hydrate and/or solvate thereof, q is 0, 1 or 2.

In an embodiment of the compounds of Formula I-X, or a pharmaceutically-acceptable salt, hydrate and/or solvate thereof, R⁴ is as defined in any one of numbered paragraphs (9) to (11).

In an embodiment of the compounds of Formula I-X, or a pharmaceutically-acceptable salt, hydrate and/or solvate thereof, R⁵ is as defined in any one of numbered paragraphs (27) to (30), or two R⁵ groups located on adjacent carbon atoms are linked, such that when taken in combination with the atoms to which they are attached, they form a fused benzene ring that is optionally substituted with one or more groups R⁵ as defined in any one of numbered paragraphs (27) to (30).

In an embodiment of the compounds of Formula I-X, or a pharmaceutically-acceptable salt, hydrate and/or solvate thereof:

R⁴ is as defined in any one of numbered paragraphs (9) to (11); and
R⁵ is as defined in any one of numbered paragraphs (27) to (30), or two R⁵ groups located on adjacent carbon atoms are linked, such that when taken in combination with the atoms to which they are attached, they form a fused benzene ring that is optionally substituted with one or more groups R⁵ as defined in any one of numbered paragraphs (27) to (30).

In a particularly suitable embodiment, the compound of Formula I has a structure according to any one of the following, or is a pharmaceutically acceptable salt, hydrate or solvate thereof:

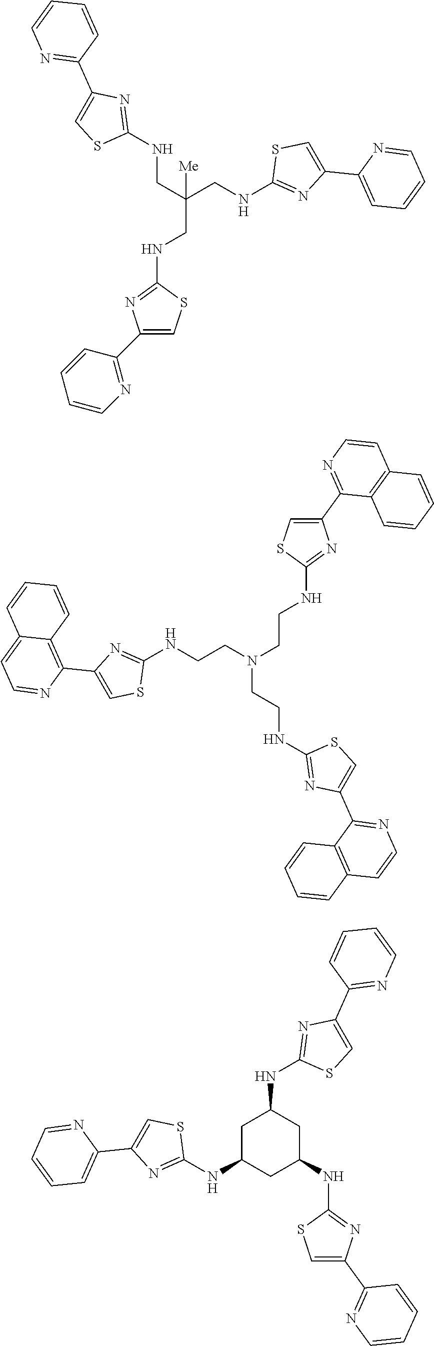

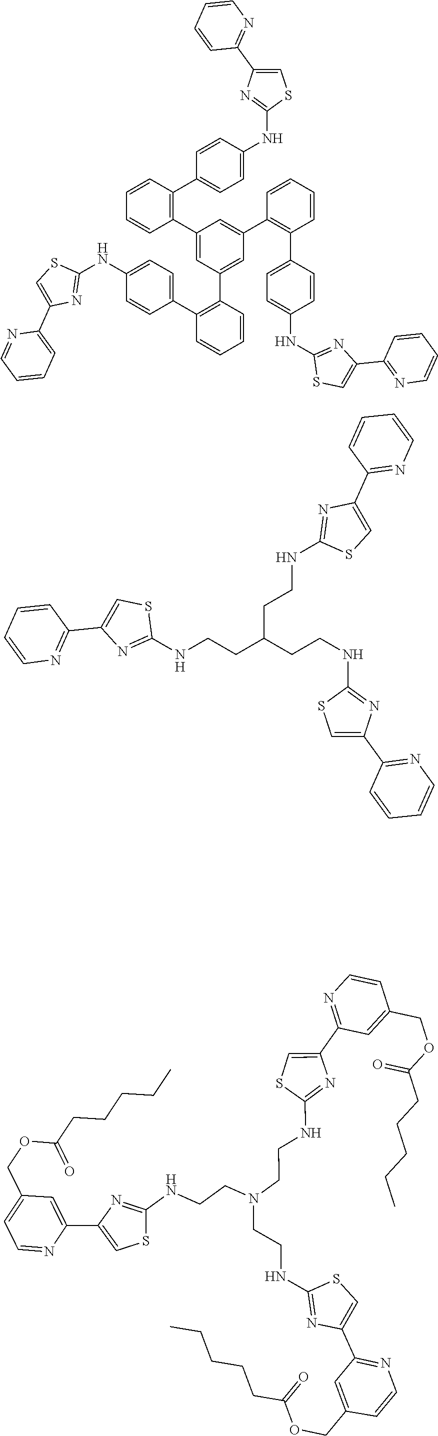

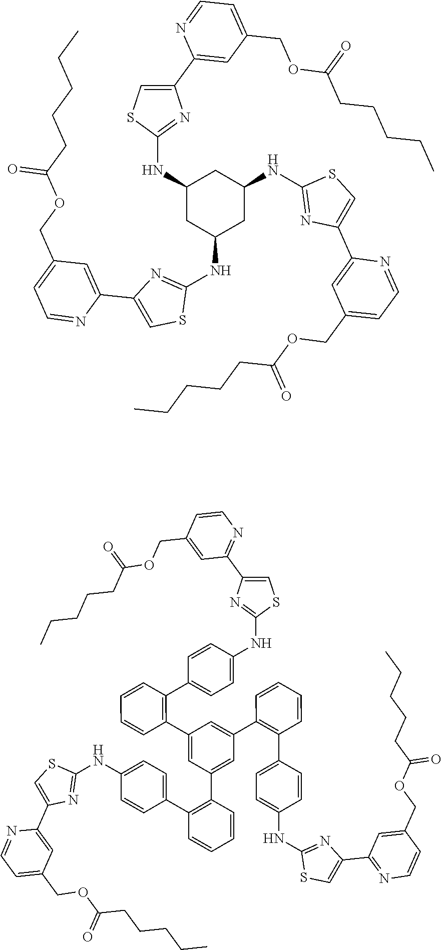 [SMILES: CCCCCC(=O)OCc1ccnc(-c2csc(N[C@H]3C[C@@H](Nc4nc(-c5cc(COC(=O)CCCCC)ccn5)cs4)C[C@@H](Nc4nc(-c5cc(COC(=O)CCCCC)ccn5)cs4)C3)n2)c1 ; CCCCCC(=O)OCc1ccnc(-c2csc(Nc3ccc(-c4ccccc4-c4cc(-c5ccccc5-c5ccc(Nc6nc(-c7cc(COC(=O)CCCCC)ccn7)cs6)cc5)cc(-c5ccccc5-c5ccc(Nc6nc(-c7cc(COC(=O)CCCCC)ccn7)cs6)cc5)c4)cc3)n2)c1]

In an embodiment, the compound of Formula I is not one of the following:

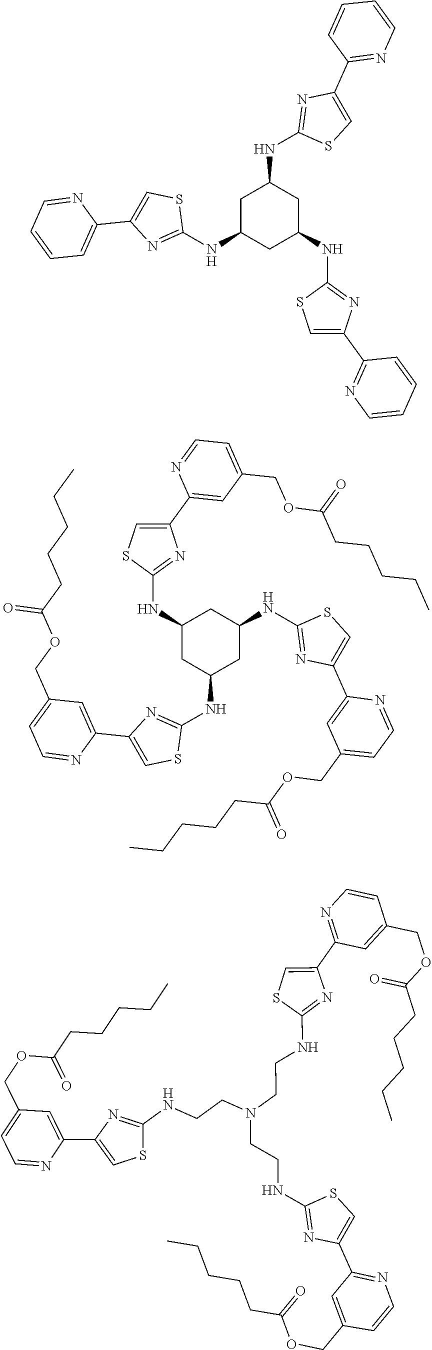 [SMILES: c1ccc(-c2csc(N[C@H]3C[C@@H](Nc4nc(-c5ccccn5)cs4)C[C@@H](Nc4nc(-c5ccccn5)cs4)C3)n2)nc1 ; CCCCCC(=O)OCc1ccnc(-c2csc(N[C@H]3C[C@@H](Nc4nc(-c5cc(COC(=O)CCCCC)ccn5)cs4)C[C@@H](Nc4nc(-c5cc(COC(=O)CCCCC)ccn5)cs4)C3)n2)c1 ; CCCCCC(=O)OCc1ccnc(-c2csc(NCCN(CCNc3nc(-c4cc(COC(=O)CCCCC)ccn4)cs3)CCNc3nc(-c4cc(COC(=O)CCCCC)ccn4)cs3)n2)c1]

### Compounds of Formula II

In another aspect, the present invention provides a compound having a structure according to Formula II shown below, or a pharmaceutically acceptable salt, hydrate or solvate thereof:

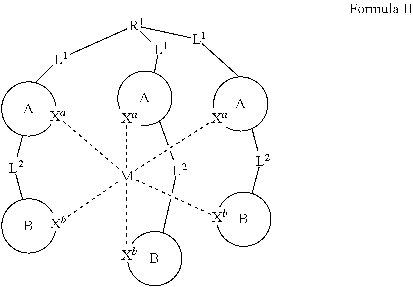

wherein
M is selected from the group consisting of Zn²⁺, Mn²⁺, Cu²⁺, Fe²⁺, CO²⁺ and Ni²⁺; and
R¹, L¹, A, X^{a}, L², B, X^{b} and any associated subgroups are as defined hereinbefore in relation to the compounds of Formula I.

As described hereinbefore, the inventors have surprisingly discovered that compounds of Formula I described herein function as tripodal ligands that are able to self-assemble in the presence of certain metals to form mononuclear complexes, i.e. compounds of Formula II.

Accordingly, it will be appreciated that R¹, L¹, A, X^{a}, L², B, X^{b} and any associated subgroups may have any of those definitions outlined hereinbefore in relation to the compounds of Formula I (including sub-formulae I-I to I-X). Suitable and preferred definitions of R¹, L¹, A, X^{a}, L², B, X^{b} in the context of compounds of Formula I are therefore suitable and preferred features definitions of R¹, L¹, A, X^{a}, L², B, X^{b} in the context of compounds of Formula II.

It will be understood that the compounds of Formula II, which are positively charged complexes, exist in association with one or more charge balancing anions. Any suitable charge balancing anions may be used.

In a particular embodiment, the compound, pharmaceutically acceptable salt, hydrate or solvate of Formula II is not Compound IIa shown below, or a pharmaceutically acceptable salt, hydrate or solvate thereof:

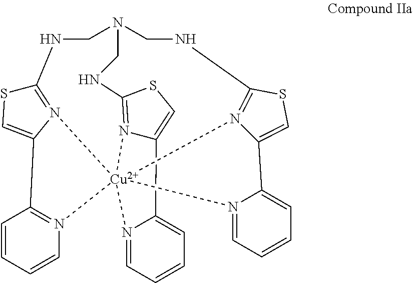

In an embodiment, M is selected from the group consisting of Zn²⁺, Mn²⁺, Fe²⁺, CO²⁺ and Ni²⁺. Suitably, M is selected from the group consisting of Zn²⁺, Mn²⁺ and Fe²⁺. Most suitably, M is selected from the group consisting of Zn²⁺ and Mn²⁺.

In an embodiment, M is selected from the group consisting of Zn²⁺, Cu²⁺, Mn²⁺, Fe²⁺, CO²⁺ and Ni²⁺. Suitably, M is selected from the group consisting of Zn²⁺, Cu²⁺, Mn²⁺ and Fe²⁺. Most suitably, M is selected from the group consisting of Zn²⁺, Cu²⁺ and Mn²⁺.

M is associated with X^{a} and X^{b} (shown in Formula II as “- - - -”). It will, however, be understood that depending on the nature of M, M may not be associated with all instances of X^{a} and X^{b}. For example, oxophilic and hard metal cations (e.g. M=Mn²⁺) may only be associated with 2 X^{a} and 2 X^{b}, with their coordination sphere being completed by complexed solvent, such as water. It will be understood that such solvated forms (including hydrates) of Formula II are within the scope of the invention.

In a particular embodiment, the compound has a structure according to either of the following:

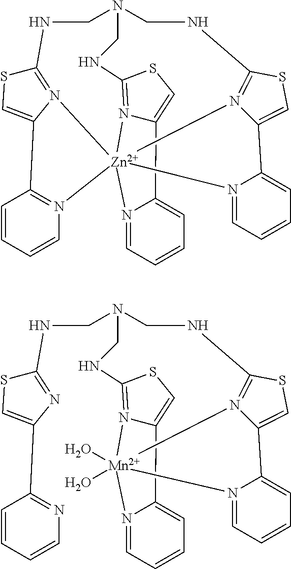

or a pharmaceutically-acceptable salt, hydrate and/or solvate thereof.

In an embodiment, when M is Cu²⁺, R¹, L¹, A, X^{a}, L², B, X^{b}, and any associated subgroups, do not form a compound having a structure:

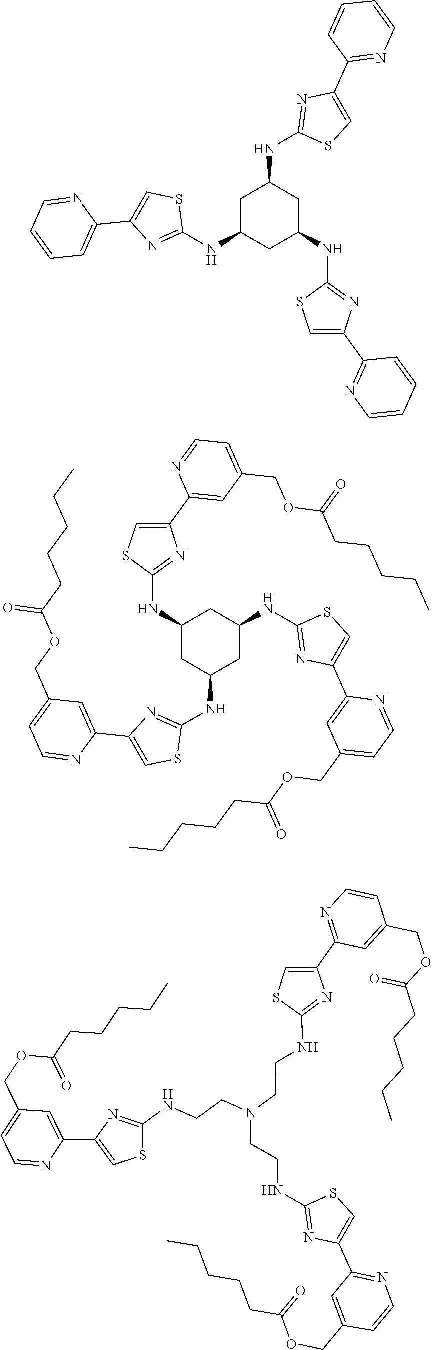 [SMILES: c1ccc(-c2csc(N[C@H]3C[C@@H](Nc4nc(-c5ccccn5)cs4)C[C@@H](Nc4nc(-c5ccccn5)cs4)C3)n2)nc1 ; CCCCCC(=O)OCc1ccnc(-c2csc(N[C@H]3C[C@@H](Nc4nc(-c5cc(COC(=O)CCCCC)ccn5)cs4)C[C@@H](Nc4nc(-c5cc(COC(=O)CCCCC)ccn5)cs4)C3)n2)c1 ; CCCCCC(=O)OCc1ccnc(-c2csc(NCCN(CCNc3nc(-c4cc(COC(=O)CCCCC)ccn4)cs3)CCNc3nc(-c4cc(COC(=O)CCCCC)ccn4)cs3)n2)c1]

In an embodiment, R¹, L¹, A, X^{a}, L², B, X^{b}, and any associated subgroups, do not form a compound having a structure:

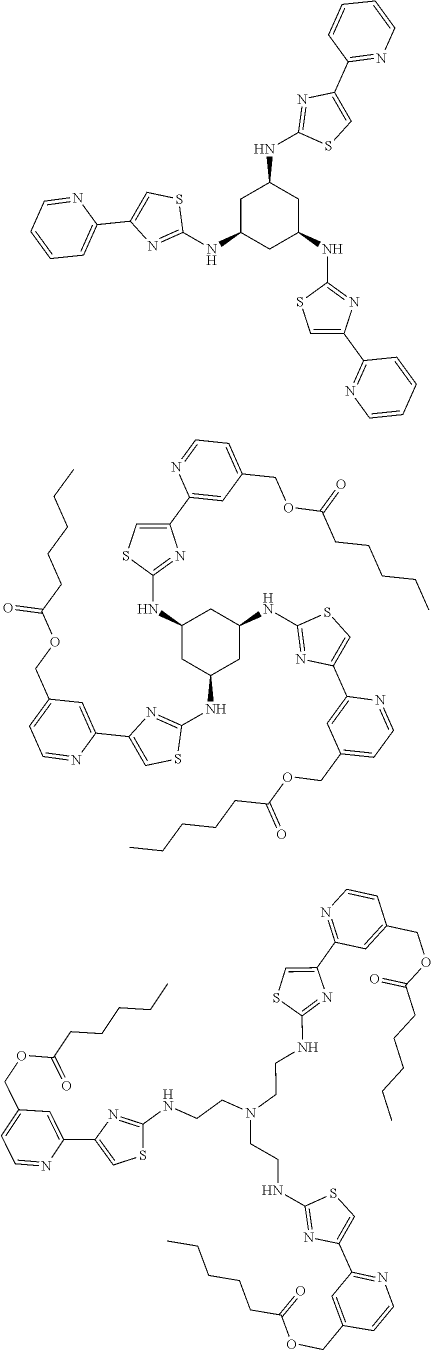 [SMILES: c1ccc(-c2csc(N[C@H]3C[C@@H](Nc4nc(-c5ccccn5)cs4)C[C@@H](Nc4nc(-c5ccccn5)cs4)C3)n2)nc1 ; CCCCCC(=O)OCc1ccnc(-c2csc(N[C@H]3C[C@@H](Nc4nc(-c5cc(COC(=O)CCCCC)ccn5)cs4)C[C@@H](Nc4nc(-c5cc(COC(=O)CCCCC)ccn5)cs4)C3)n2)c1 ; CCCCCC(=O)OCc1ccnc(-c2csc(NCCN(CCNc3nc(-c4cc(COC(=O)CCCCC)ccn4)cs3)CCNc3nc(-c4cc(COC(=O)CCCCC)ccn4)cs3)n2)c1]

### Compounds of Formula III

In another aspect, the present invention provides a compound having a structure according to Formula III shown below, or a pharmaceutically acceptable salt, hydrate or solvate thereof:

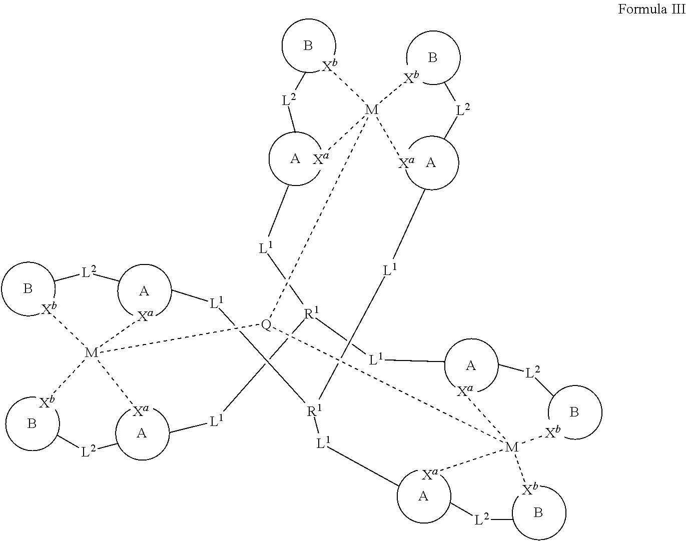

wherein
M is selected from the group consisting of Zn²⁺, Mn²⁺, Cu²⁺, Fe²⁺, Co²⁺ and Ni²⁺;
Q is an anion selected from the group consisting of spherical monoanionic anions, trigonal planar anions, dianionic tetrahedral anions, trianionic tetrahedral anions, dianionic octahedral anions and trianionic octahedral anions; and
R¹, L¹, A, X^{a}, L², B, X^{b} and any associated subgroups are as defined hereinbefore in relation to the compounds of Formula I;
with the proviso that the compound does not have a structure according to Formula IIIa:

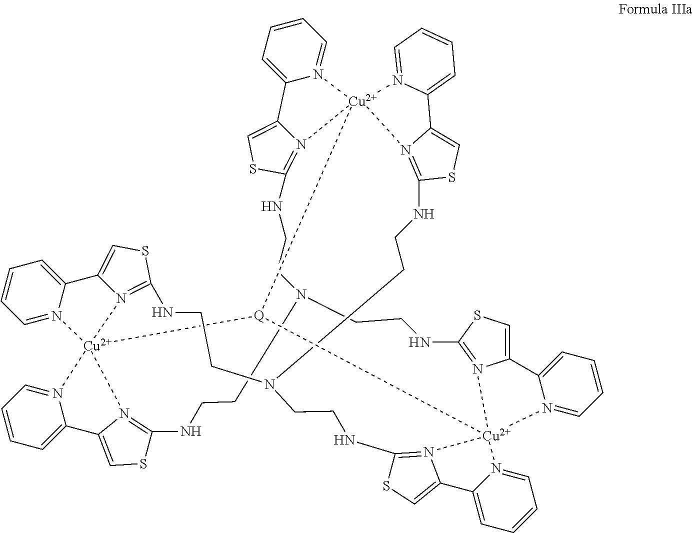

in which Q is Br⁻, I⁻, CO₃²⁻, SiF₆²⁻, IO₆³⁻, VO₄³⁻, WO₄²⁻, CrO₄²⁻, SO₄²⁻, HSO₄⁻, AsO₄³⁻, PO₄³⁻ or HPO₄²⁻.

As described hereinbefore, the inventors have surprisingly discovered that compounds of Formula I described herein function as tripodal ligands that are able to self-assemble in the presence of certain metals and anions to form trinuclear complexes, i.e. compounds of Formula III.

Accordingly, it will be appreciated that R¹, L¹, A, X^{a}, L², B, X^{b} and any associated subgroups may have any of those definitions outlined hereinbefore in relation to the compounds of Formula I (including sub-formulae I-I to I-X). Suitable and preferred definitions of R¹, L¹, A, X^{a}, L², B, X^{b} in the context of compounds of Formula I are therefore suitable and preferred features definitions of R¹, L¹, A, X^{a}, L², B, X^{b} in the context of compounds of Formula III.

It will be understood that the compounds of Formula III, which are positively charged complexes, exist in association with one or more charge balancing anions. Any suitable charge balancing anions may be used.

In an embodiment, M is selected from the group consisting of Zn²⁺, Mn²⁺, Fe²⁺, CO²⁺ and Ni²⁺. Suitably, M is selected from the group consisting of Zn²⁺, Mn²⁺ and Fe²⁺. Most suitably, M is selected from the group consisting of Zn²⁺ and Mn²⁺.

In an embodiment, M is selected from the group consisting of Zn²⁺, Cu²⁺, Mn²⁺, Fe²⁺, CO²⁺ and Ni²⁺. Suitably, M is selected from the group consisting of Zn²⁺, Cu²⁺, Mn²⁺ and Fe²⁺. Most suitably, M is selected from the group consisting of Zn²⁺, Cu²⁺ and Mn²⁺.

M is associated with X^{a} and X^{b} (shown in Formula III as “- - - -”). It will, however, be understood that depending on the nature of M, M may additionally be associated with complexed solvent. For example, oxophilic and hard metal cations (e.g. M=Mn²⁺) may have their coordination sphere completed by complexed solvent, such as water. It will be understood that such solvated forms (including hydrates) of Formula III are within the scope of the invention.

In an embodiment, Q is Br⁻, I⁻, CO₃²⁻, SiF₆²⁻, IO₆³⁻, VO₄³⁻, WO₄²⁻, CrO₄²⁻, SO₄²⁻, HSO₄⁻, AsO₄³⁻, PO₄³⁻ or HPO₄²⁻.

In an embodiment, Q is an anion selected from dianionic tetrahedral anions and trianionic tetrahedral anions. Suitably, Q is an anion selected from dianionic tetrahedral oxoanions and trianionic tetrahedral oxoanions.

In a particular embodiment, Q is sulfate (SO₄²⁻), phosphate (PO₄³⁻) or organophosphate (RPO₄²⁻). Particular, non-limiting examples of organophosphates include monophenylphosphate.

In a particular embodiment, the compound has a structure according to any of the following:

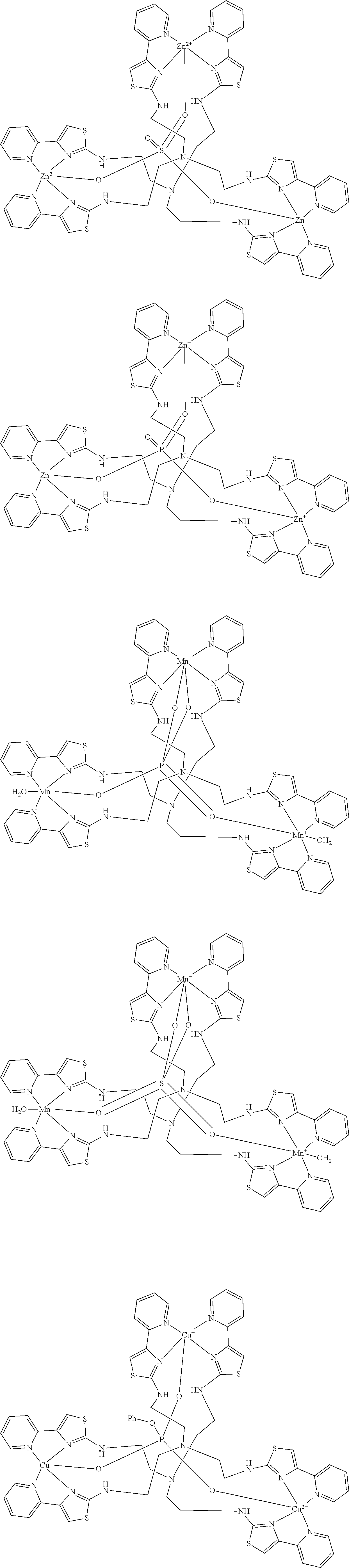

or a pharmaceutically-acceptable salt, hydrate and/or solvate thereof.

In an embodiment, when M is Cu²⁺, R¹, L¹, A, X^{a}, L², B, X^{b}, and any associated subgroups, do not form a compound having a structure:

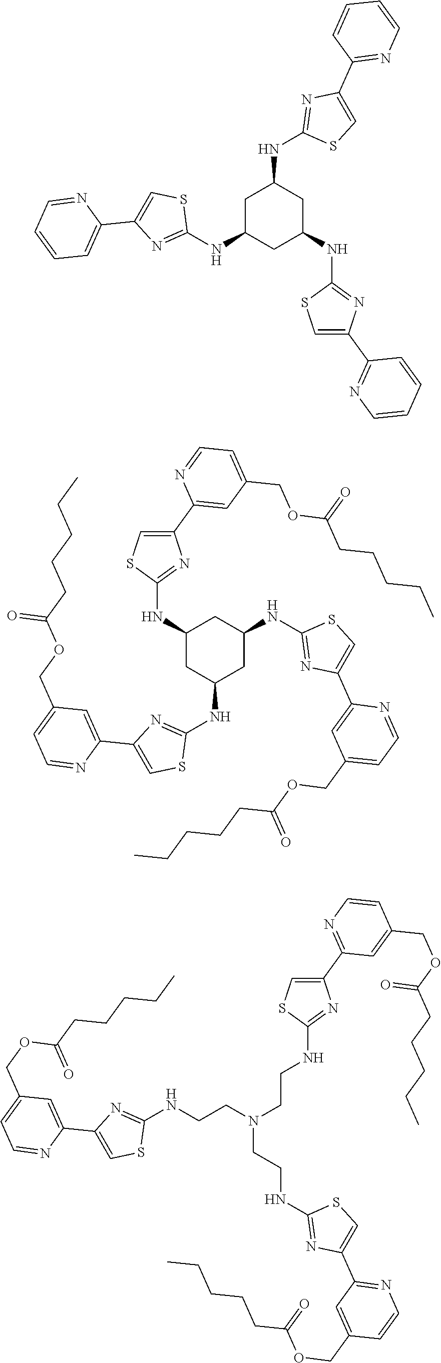 [SMILES: c1ccc(-c2csc(N[C@H]3C[C@@H](Nc4nc(-c5ccccn5)cs4)C[C@@H](Nc4nc(-c5ccccn5)cs4)C3)n2)nc1 ; CCCCCC(=O)OCc1ccnc(-c2csc(N[C@H]3C[C@@H](Nc4nc(-c5cc(COC(=O)CCCCC)ccn5)cs4)C[C@@H](Nc4nc(-c5cc(COC(=O)CCCCC)ccn5)cs4)C3)n2)c1 ; CCCCCC(=O)OCc1ccnc(-c2csc(NCCN(CCNc3nc(-c4cc(COC(=O)CCCCC)ccn4)cs3)CCNc3nc(-c4cc(COC(=O)CCCCC)ccn4)cs3)n2)c1]

In an embodiment, R¹, L¹, A, X^{a}, L², B, X^{b}, and any associated subgroups, do not form a compound having a structure:

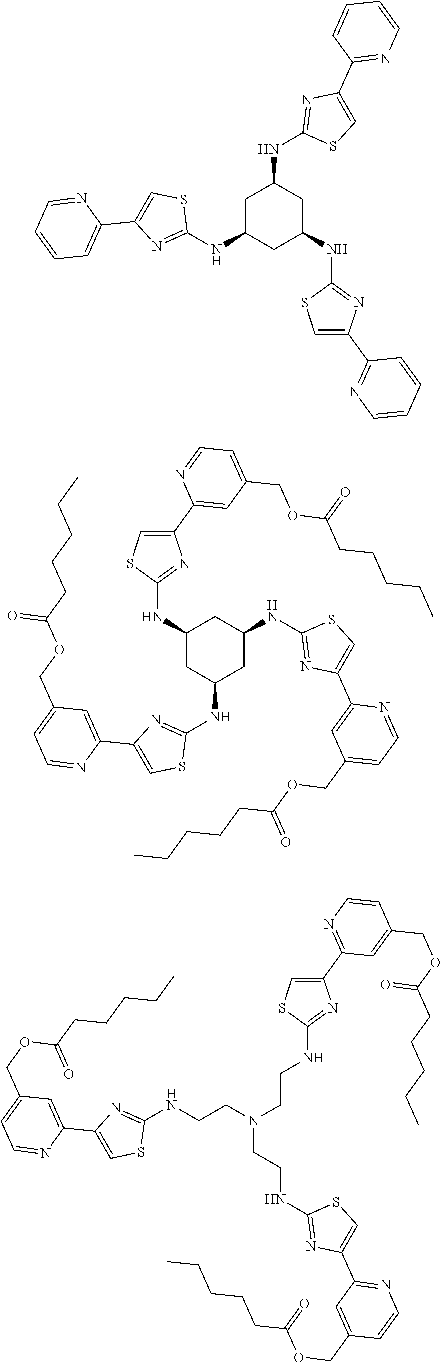 [SMILES: c1ccc(-c2csc(N[C@H]3C[C@@H](Nc4nc(-c5ccccn5)cs4)C[C@@H](Nc4nc(-c5ccccn5)cs4)C3)n2)nc1 ; CCCCCC(=O)OCc1ccnc(-c2csc(N[C@H]3C[C@@H](Nc4nc(-c5cc(COC(=O)CCCCC)ccn5)cs4)C[C@@H](Nc4nc(-c5cc(COC(=O)CCCCC)ccn5)cs4)C3)n2)c1 ; CCCCCC(=O)OCc1ccnc(-c2csc(NCCN(CCNc3nc(-c4cc(COC(=O)CCCCC)ccn4)cs3)CCNc3nc(-c4cc(COC(=O)CCCCC)ccn4)cs3)n2)c1]

The various functional groups and substituents making up the compounds of the invention, are typically chosen such that the molecular weight of the compound does not exceed 1000. More usually, the molecular weight of the compound will be less than 900, for example less than 800, or less than 750, or less than 700, or less than 650. More preferably, the molecular weight is less than 600 and, for example, is 550 or less.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulfuric, phosphoric, trifluoroacetic, formic, citric methane sulfonate or maleic acid. In addition, a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, trimethylamine, piperidine, morpholine or tris-(2-hydroxyethyl)amine.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of the invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E- and Z-isomers).

It is to be understood that the present invention encompasses all optical, diastereoisomers and geometric isomers of the compounds of the invention and mixtures thereof that possess activity.

The present invention also encompasses compounds of the invention as defined herein which comprise one or more isotopic substitutions. For example, H may be in any isotopic form, including 1H, 2H(D), and 3H (T); C may be in any isotopic form, including 12C, 13C, and 14C; and O may be in any isotopic form, including 16O and 18O; and the like.

It is also to be understood that certain compounds of the invention may exist in solvated as well as unsolvated forms such as, for example, hydrated forms. It is to be understood that the invention encompasses all such solvated forms that possess activity.

It is also to be understood that certain compounds of the invention may exhibit polymorphism, and that the invention encompasses all such forms that possess activity.

Compounds of the invention may exist in a number of different tautomeric forms and references to compounds of the invention include all such forms. For the avoidance of doubt, where a compound can exist in one of several tautomeric forms, and only one is specifically described or shown, all others are nevertheless embraced by Formula I, II or III (or sub-formulae thereof). Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

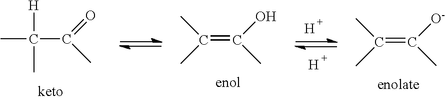

Compounds of the invention containing an amine function may also form N-oxides. A reference herein to a compound of the invention that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (mCPBA), for example, in an inert solvent such as dichloromethane.

The compounds of the invention may be administered in the form of a pro-drug which is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the invention, and in-vivo cleavable amide derivatives that may be formed at a carboxy group or an amino group in a compound of the invention.

Accordingly, the present invention includes those compounds of the invention as defined hereinbefore, when made available by organic synthesis and when made available within the human or animal body by way of cleavage of a pro-drug thereof. Accordingly, the present invention includes those compounds of the invention that are produced by organic synthetic means and also such compounds that are produced in the human or animal body by way of metabolism of a precursor compound. The compounds of the invention may be synthetically-produced compounds or metabolically-produced compounds.

A suitable pharmaceutically acceptable pro-drug of a compound of the invention is one that is based on reasonable medical judgement as being suitable for administration to the human or animal body without undesirable pharmacological activities and without undue toxicity.

Various forms of pro-drug have been described, for example in the following documents:—
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically acceptable pro-drug of a compound of the invention that possesses a carboxy group is, for example, an in vivo cleavable ester thereof. An in vivo cleavable ester of a compound of the invention containing a carboxy group is, for example, a pharmaceutically acceptable ester which is cleaved in the human or animal body to produce the parent acid or parent alcohol. Suitable pharmaceutically acceptable esters for carboxy include (1-6C)alkyl esters such as methyl, ethyl and tert-butyl, (1-6C)alkoxymethyl esters such as methoxymethyl esters, (1-6C)alkanoyloxymethyl esters such as pivaloyloxymethyl esters, 3-phthalidyl esters, (3-8C)cycloalkylcarbonyloxy-(1-6C)alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and (1-6C)alkoxycarbonyloxy-(1-6C)alkyl esters such as methoxycarbonyloxymethyl and 1-methoxycarbonyloxyethyl esters.

A suitable pharmaceutically acceptable pro-drug of a compound of the invention that possesses a hydroxy group is, for example, an in vivo cleavable ester or ether thereof. An in vivo cleavable ester or ether of a compound of the invention containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include (1-10C)alkanoyl groups such as acetyl, benzoyl, phenylacetyl and substituted benzoyl and phenylacetyl groups, (1-10C)alkoxycarbonyl groups such as ethoxycarbonyl, N,N-(1-6C)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(1-4C)alkylpiperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable pro-drug of a compound of the invention that possesses a carboxy group is, for example, an in vivo cleavable amide thereof, for example an amide formed with an amine such as ammonia, a (1-4C)alkylamine such as methylamine, a [(1-4C)alkyl]₂amine such as dimethylamine, N-ethyl-N-methylamine or diethylamine, a (1-4C)alkoxy-(2-4C)alkylamine such as 2-methoxyethylamine, a phenyl-(1-4C)alkylamine such as benzylamine and amino acids such as glycine or an ester thereof.

A suitable pharmaceutically acceptable pro-drug of a compound of the invention that possesses an amino group is, for example, an in vivo cleavable amide derivative thereof. Suitable pharmaceutically acceptable amides from an amino group include, for example an amide formed with (1-10C)alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(1-4C)alkyl)piperazin-1-ylmethyl.

The in vivo effects of a compound of the invention may be exerted in part by one or more metabolites that are formed within the human or animal body after administration of a compound of invention. As stated hereinbefore, the in vivo effects of a compound of the invention may also be exerted by way of metabolism of a precursor compound (a pro-drug).

Though the present invention may relate to any compound or particular group of compounds defined herein by way of optional, preferred or suitable features or otherwise in terms of particular embodiments, the present invention may also relate to any compound or particular group of compounds that specifically excludes said optional, preferred or suitable features or particular embodiments.

Suitably, the present invention excludes any individual compounds not possessing the biological activity defined herein.

### Synthesis

The compounds of the invention can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

It will be appreciated that during the synthesis of the compounds of the invention in the processes defined herein, or during the synthesis of certain starting materials, it may be desirable to protect certain substituent groups to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

Resins may also be used as a protecting group.

The methodology employed to synthesise a compound of the invention will vary depending on the nature of R¹, L¹, A, X^{a}, L², B, X^{b}, M, Q and any substituent groups or subgroups associated therewith. Suitable processes for their preparation are described further in the accompanying Examples.

Once a compound of the invention has been synthesised by any one of the processes defined herein, the processes may then further comprise the additional steps of:
(i) removing any protecting groups present;
(ii) converting the compound of the invention into another compound of the invention;
(iii) forming a pharmaceutically acceptable salt, hydrate or solvate thereof; and/or
(iv) forming a prodrug thereof.

An example of (ii) above is when a compound of the invention is synthesised and then one or more of the groups R¹, L¹, A, X^{a}, L², B, X^{b}, M and Q may be further reacted to change the nature of the group and provide an alternative compound of the invention.

The resultant compounds of the invention can be isolated and purified using techniques well known in the art.

The compounds of the invention may be synthesised by the synthetic routes shown in the Examples section below.

### Biological Activity

The biological assays described in the Examples section herein may be used to measure the pharmacological effects of the compounds of the invention.

Although the pharmacological properties of the compounds of the invention vary with structural change, as expected, the compounds of the invention were found to be selectively active towards a range of human cancer cells compared to healthy, non-cancerous cells, according to the in vitro assays described in the Examples section.

### Pharmaceutical Compositions

According to a further aspect of the present invention there is provided a pharmaceutical composition comprising a compound having a structure according to Formula I as defined herein, or a pharmaceutically acceptable salt, hydrate or solvate thereof, a source of M as defined herein, and one or more pharmaceutically acceptable diluents, excipients or carriers. Suitably, the pharmaceutical composition further comprises a source of Q as defined herein.

According to a further aspect of the present invention there is provided a pharmaceutical composition comprising a compound having a structure according to Formula II as defined herein, or a pharmaceutically acceptable salt, hydrate or solvate thereof, and one or more pharmaceutically acceptable diluents, excipients or carriers. Suitably, the pharmaceutical composition further comprises a source of Q as defined herein.

According to a further aspect of the present invention there is provided a pharmaceutical composition comprising a compound having a structure according to Formula III as defined herein or a pharmaceutically acceptable salt, hydrate or solvate thereof, and one or more pharmaceutically acceptable diluents, excipients or carriers.

For the avoidance of doubt, it will be understood that pharmaceutical compositions comprising compounds of Formula I extend to (i.e. encompass) pharmaceutical compositions comprising compound Ia defined herein.

For the avoidance of doubt, it will be understood that pharmaceutical compositions comprising compounds of Formula II extend to (i.e. encompass) pharmaceutical compositions comprising compound IIa defined herein.

For the avoidance of doubt, it will be understood that pharmaceutical compositions comprising compounds of Formula III extend to (i.e. encompass) pharmaceutical compositions comprising compounds of Formula IIIa defined herein in which Q is Br⁻, I⁻, CO₃²⁻, SiF₆²⁻, IO₆³⁻, VO₄³⁻, WO₄²⁻, CrO₄²⁻, SO₄²⁻, HSO₄⁻, AsO₄³⁻, PO₄³⁻ or HPO₄²⁻.

It will be understand that any pharmaceutically acceptable source of M may be used, examples of which will be familiar to the skilled person. For example, the source of M may be an organic salt (e.g. the salt of M and a carboxylic acid, such as M acetate or M propionate, or the salt of M and an organosulfonic acid, such as M trifluoromethanesulfonate) or an inorganic salt (e.g. M perchlorate, M nitrate, M tetrafluoroborate). In a particular embodiment, the source of M is M acetate.

It will be understand that any pharmaceutically acceptable source of Q may be used, examples of which will be familiar to the skilled person. For example, the source of Q may be organic, such Et₃NX, where X is a halide (i.e. a spherical monoanionic anion) or Bu₄NH₂PO₄ (where PO₄³⁻ is a trianionic tetrahedral anion) or Bu₄NHSO₄ (where SO₄²⁻ is a dianionic tetrahedral anion). Alternatively, the source of Q may be inorganic, such as Na₂SiF₆ (where SiF₆²⁻ is a dianionic octahedral anion) or Na₂O₃POPh (where PhPO₄²⁻ is a dianionic tetrahedral anion).

It will be understood that the source of M may also be the source of Q (e.g. ZnSO₄). In a particular embodiment, the source of M is also the source of Q.

The pharmaceutical compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The pharmaceutical compositions of the invention may be obtained by conventional procedures using conventional pharmaceutical excipients, well known in the art. Thus, compositions intended for oral use may contain, for example, one or more colouring, sweetening, flavouring and/or preservative agents.

The pharmaceutical compositions of the invention comprise an effective amount of a compound of the invention, the source of M and source of Q, as required. An effective amount of a compound of the present invention for use in therapy is an amount sufficient to treat or prevent a proliferative condition referred to herein, slow its progression and/or reduce the symptoms associated with the condition.

The amount of active ingredient that is combined with one or more excipients to produce a single dosage form will necessarily vary depending upon the individual treated and the particular route of administration. For example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the invention will naturally vary according to the nature and severity of the conditions, the age and sex of the animal or patient and the route of administration, according to well-known principles of medicine.

In using a compound of the invention for therapeutic or prophylactic purposes it will generally be administered so that a daily dose in the range, for example, 0.1 mg/kg to 75 mg/kg body weight is received, given if required in divided doses. In general lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration may also be suitable, particularly in tablet form. Typically, unit dosage forms will contain about 0.5 mg to 0.5 g of a compound of this invention.

### Therapeutic Uses and Applications

The present invention provides compounds that are active against a range of human cancer cell lines. The compounds of the invention also show remarkable selective activity towards cancer cell lines compared to non-cancerous cells. Without wishing to be bound by theory, the inventors believe that the activity of the compound is attributable to their ability to bind phosphate anions and/or hydrolyse phosphate esters. The compounds of the invention also exhibit selective inhibition of a number of kinases.

The present invention therefore provides a compound of Formula I as defined herein, or a pharmaceutically acceptable salt, hydrate or solvate thereof, in combination with a source of M as defined herein, for use as a medicament. Suitably, the compound of Formula I and the source of M are used in further combination with a source of Q as defined herein.

The present invention also provides a compound of Formula I as defined herein, or a pharmaceutically acceptable salt, hydrate or solvate thereof, in combination with a source of M as defined herein, for use in the treatment of a proliferative disorder (e.g. cancer). Suitably, the compound of Formula I and the source of M are in further combination with a source of Q as defined herein.

The present invention also provides a method of treating a proliferative disorder (e.g. cancer) in a patient in need of such treatment, the method comprising administering to the patient a therapeutically effective amount of a compound of Formula I as defined herein, or a pharmaceutically acceptable salt, hydrate or solvate thereof, in combination with a source of M as defined herein. Suitably, the compound of Formula I and the source of M are administered in combination with a source of Q.

The present invention also provides the use of a compound of Formula I, or a pharmaceutically acceptable salt, hydrate or solvate thereof, in the manufacture of a medicament. Suitably, the medicament is for the treatment of a proliferative disorder (e.g. cancer).

As described hereinbefore, the compounds of Formula I demonstrate the ability to self-assemble with M to form the mononuclear compounds of Formula II. When contacted with a suitable anion, either in vitro or in vivo (such as a biological source of phosphate), the compounds of Formula II can be readily converted into the trinuclear compounds of Formula III. The data presented herein show that the compounds of Formula II and III are active against a range of human cancer cell lines and/or inhibit of a number of kinases.. Thus, it will be understood that in the therapeutic uses of the invention involving compounds of Formula I, said compound is used in combination with a source of M. Suitably, the compound of Formula I and the source of M are used in combination with a source of Q. It will be understood that simultaneous, separate or sequential use (e.g. administration) of the compound of Formula I, the source of M and optionally the source of Q are encompassed.

Exemplary definitions of M and Q, as well as sources of them, are discussed hereinbefore.

For the avoidance of doubt, therapeutic uses of compounds of Formula I extend to (i.e. encompass) the therapeutic uses of compound Ia shown below:

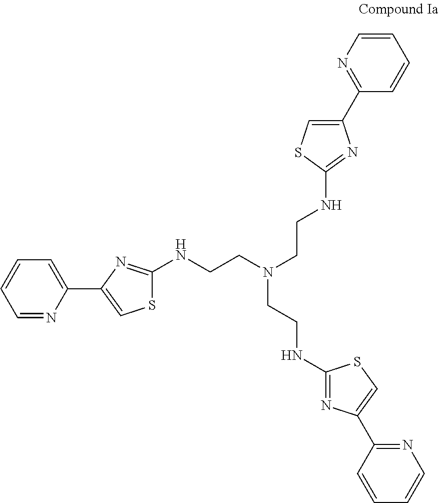 [SMILES: c1ccc(-c2csc(NCCN(CCNc3nc(-c4ccccn4)cs3)CCNc3nc(-c4ccccn4)cs3)n2)nc1]

The present invention also provides a compound of Formula II as defined herein, or a pharmaceutically acceptable salt, hydrate or solvate thereof, for use as a medicament. Suitably, the compound of Formula II is in combination with a source of Q as defined herein.

The present invention also provides a compound of Formula II as defined herein, or a pharmaceutically acceptable salt, hydrate or solvate thereof, for use in the treatment of a proliferative disorder (e.g. cancer). Suitably, the compound of Formula II is in combination with a source of Q as defined herein.

The present invention also provides a method of treating a proliferative disorder (e.g. cancer) in a patient in need of such treatment, the method comprising administering to the patient a therapeutically effective amount of a compound of Formula II as defined herein, or a pharmaceutically acceptable salt, hydrate or solvate thereof. Suitably, the compound of Formula II is administered in combination with a source of Q.

The present invention also provides the use of a compound of Formula II, or a pharmaceutically acceptable salt, hydrate or solvate thereof, in the manufacture of a medicament. Suitably, the medicament is for the treatment of a proliferative disorder (e.g. cancer).

As described herein, compounds of Formula II exhibit selective activity against a range of human cancer cell lines and/or inhibition of a number of kinases.

In a particular embodiment, the compound of Formula II is used in combination with a source of Q. As described herein, compounds of Formula II, when contacted with a source of Q (either in vitro or in vivo), undergo a self-assembly process to yield compounds of Formula III, which exhibit selective activity against a range of human cancer cell lines and/or inhibition of a number of kinases. It will be understood that this embodiment encompasses simultaneous, separate or sequential use (e.g. administration) of the compound of Formula II and the source of Q.

Exemplary definitions of M and Q, as well as sources of them, are discussed hereinbefore.

For the avoidance of doubt, therapeutic uses of compounds of Formula II extend to (i.e. encompass) the therapeutic uses of Compound IIa shown below:

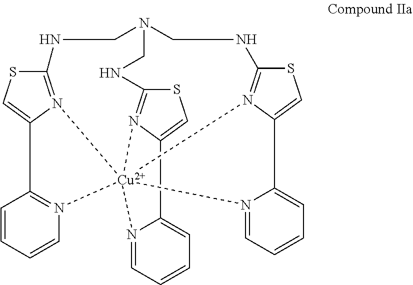

The present invention also provides a compound of Formula III as defined herein, or a pharmaceutically acceptable salt, hydrate or solvate thereof, for use as a medicament.

The present invention also provides a compound of Formula III as defined herein, or a pharmaceutically acceptable salt, hydrate or solvate thereof, for use in the treatment of a proliferative disorder (e.g. cancer).

The present invention also provides a method of treating a proliferative disorder (e.g. cancer) in a patient in need of such treatment, the method comprising administering to the patient a therapeutically effective amount of a compound of Formula III as defined herein, or a pharmaceutically acceptable salt, hydrate or solvate thereof.

The present invention also provides the use of a compound of Formula III, or a pharmaceutically acceptable salt, hydrate or solvate thereof, in the manufacture of a medicament. Suitably, the medicament is for the treatment of a proliferative disorder (e.g. cancer).

As described herein, compounds of Formula III exhibit selective activity against a range of human cancer cell lines and/or inhibition of a number of kinases.

Exemplary definitions of M and Q, as well as sources of them, are discussed hereinbefore.

For the avoidance of doubt, therapeutic uses of compounds of Formula III extend to (i.e. encompass) the therapeutic uses of compounds of Formula IIIa shown below:

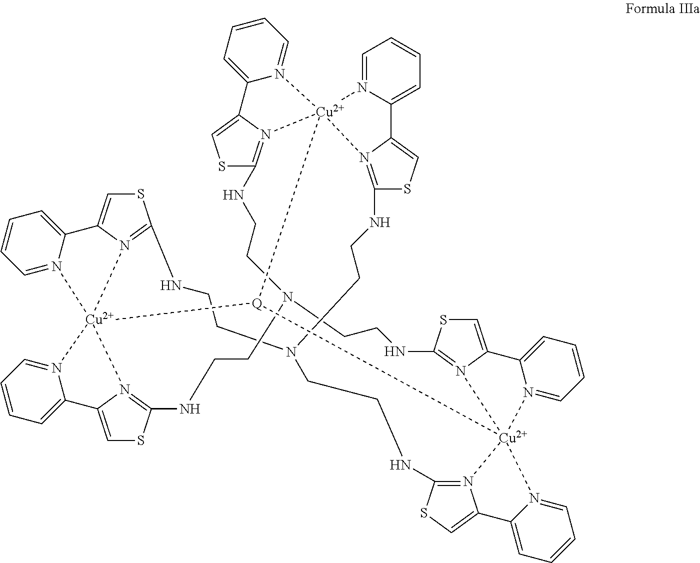

in which Q is Br⁻, I⁻, CO₃²⁻, SiF₆²⁻, IO₆³⁻, VO₄³⁻, WO₄²⁻, CrO₄²⁻, SO₄²⁻, HSO₄⁻, AsO₄³⁻, PO₄³⁻ or HPO₄²⁻.

The present invention also provides a pharmaceutical composition as defined herein, or a pharmaceutically acceptable salt, hydrate or solvate thereof, for use as a medicament.

The present invention also provides a pharmaceutical composition as defined herein, or a pharmaceutically acceptable salt, hydrate or solvate thereof, for use in the treatment of a proliferative disorder (e.g. cancer).

The present invention also provides a method of treating a proliferative disorder (e.g. cancer) in a patient in need of such treatment, the method comprising administering to the patient a therapeutically effective amount of a pharmaceutical composition as defined herein.

The term “proliferative disorder”, “proliferative condition” and “proliferative disease” are used interchangeably herein and pertain to an unwanted or uncontrolled cellular proliferation of excessive or abnormal cells which is undesired, such as, neoplastic or hyperplastic growth, whether in vitro or in vivo.

In the above-outlined aspects of the invention, the proliferative disorder is suitably cancer, and the cancer is suitably a human cancer. In particular, the compounds of the present invention will be useful for the treatment of any cancer in which a mis-match repair inhibition is beneficial. Any suitable cancer may be targeted (e.g. adenoid cystic carcinoma, adrenal gland tumor, amyloidosis, anal cancer, appendix cancer, astrocytoma, ataxia-telangiectasia, Beckwith-Wiedemann Syndrome, bile duct cancer (cholangiocarcinoma), Birt-Hogg-Dubé Syndrome, bladder cancer, bone cancer, brain stem glioma, brain tumor, breast cancer, Carney Complex, central nervous system tumors, cervical cancer, colorectal cancer, Cowden Syndrome, craniopharyngioma, desmoplastic infantile ganglioglioma, ependymoma, esophageal cancer, Ewing sarcoma, eye cancer, eyelid cancer, familial adenomatous polyposis, familial GIST, familial malignant melanoma, familial non-VHL clear cell renal cell carcinoma, familial pancreatic cancer, gallbladder cancer, gastrointestinal stromal tumor—GIST, germ cell tumor, gestational trophoblastic disease, head and neck cancer, hereditary breast and ovarian cancer, hereditary diffuse gastric cancer, hereditary leiomyomatosis and renal cell cancer, hereditary mixed polyposis syndrome, hereditary pancreatitis, hereditary papillary renal carcinoma, juvenile polyposis syndrome, kidney cancer, lacrimal gland tumor, laryngeal and hypopharyngeal cancer, leukemia (acute lymphoblastic leukamia (ALL), acute myeloid leukemia (AML), B-cell prolymphocytic leukemia, hairy cell leukemia, chronic lymphocytic leukemia (CLL), chronic myeloid leukemia (CML), chronic T-cell lymphocytic leukemia, eosinophilic leukemia), Li-Fraumeni Syndrome, liver cancer, lung cancer (non-small cell lung cancer, small cell lung cancer), Lymphoma (Hodgkin, non-Hodgkin), Lynch Syndrome, mastocytosis, medulloblastoma, melanoma, meningioma, mesothelioma, multiple endocrine neoplasia Type 1 & 2, multiple myeloma, MUTYH (or MYH)-associated polyposis, myelodysplastic syndromes (MDS), nasal cavity and paranasal sinus Cancer, nasopharyngeal Cancer, neuroblastoma, neuroendocrine tumors (e.g. of the gastrointestinal tract, lung or pancreas), neurofibromatosis Type 1 & 2, nevoid basal cell carcinoma syndrome, oral and oropharyngeal cancer, osteosarcoma, ovarian/fallopian tube/peritoneal cancer, pancreatic cancer, parathyroid cancer, penile cancer, Peutz-Jeghers Syndrome, pheochromocytoma, paraganglioma, pituitary gland tumor, pleuropulmonary blastoma, prostate cancer, retinoblastoma, rhabdomyosarcoma, salivary gland cancer, sarcoma (e.g. Kaposi or soft tissue), skin cancer, small bowel cancer, stomach cancer, testicular cancer, thymoma and thymic carcinoma, thyroid cancer, tuberous sclerosis complex, uterine cancer, vaginal cancer, Von Hippel-Lindau syndrome, vulvar cancer, Waldenstrom's macroglobulinemia, Werner syndrome, Wilms Tumor and xeroderma pigmentosum). Particular cancers of interest include haematological cancers such as lymphomas (including diffuse large B-cell lymphoma (DLBCL), follicular lymphoma (FL), Burkitt lymphoma (BL) and angioimmunoblastic T-cell lymphoma (AITL)), leukaemias (including acute lymphoblastic leukaemia (ALL) and chronic myeloid leukaemia (CML)), multiple myeloma, breast cancer, non-small cell lung cancer (NSCLC), colorectal cancer, endometrial cancer, gastro-oesophageal cancer, neuroendocrine cancers, osteosarcomas, prostate cancer, pancreatic cancer, small intestine cancer, bladder cancer, rectal cancer, cholangiocarcinoma, CNS cancer, thyroid cancer, head and neck cancer, oesophageal cancer, and ovarian cancer.

The compounds of the present invention (when used in combination with the source of M and/or Q, as required) may also be used to treat triplet diseases or disorders.

### Routes of Administration

The compounds of the invention or pharmaceutical compositions comprising these compounds may be administered to a subject by any convenient route of administration, whether systemically, peripherally or topically (i.e., at the site of desired action).

Routes of administration include, but are not limited to, oral (e.g, by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including intratumoral, subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### Combination Therapies

The compounds of the present invention (and the source of M and/or Q, as required) may be administered as a sole therapy or may involve, in addition to a compound of the invention, conventional surgery or radiotherapy or chemotherapy or a targeted agent.

Such chemotherapy or targeted agent may include one or more of the following categories:
(i) Antiproliferative/antineoplastic drugs and combinations thereof, as used in medical oncology, such as, but not limited to, alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
(ii) cytostatic agents such as, but not limited to, antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), steroid hormones, including progestogens (for example megestrol acetate) and corticosteroids (for example dexamethasone, prednisone and prednisolone), aromatase inhibitors (for example as anastrozole, letrozole, vorazole and exemestane) and inhibitors of 5α-reductase such as finasteride;
(iii) anti-invasion agents such as, but not limited to, c-Src kinase family inhibitors 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341), N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661), bosutinib (SKI-606), and metalloproteinase inhibitors such as marimastat, inhibitors of urokinase plasminogen activator receptor function or antibodies to Heparanase;
(iv) inhibitors of growth factor function such as, but not limited to, growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stern et al. (Critical reviews in oncology/haematology, 2005, Vol. 54, pp¹ 1-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib); inhibitors of the hepatocyte growth factor family; inhibitors of the insulin growth factor family; inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib (BAY 43-9006), tipifarnib (R115777) and lonafarnib (SCH66336)), inhibitors of cell signalling through MEK and/or AKT kinases, c-kit inhibitors, abl kinase inhibitors, PI3 kinase inhibitors, Plt3 kinase inhibitors, CSF-1R kinase inhibitors, IGF receptor (insulin-like growth factor) kinase inhibitors; aurora kinase inhibitors and cyclin dependent kinase inhibitors such as CDK2 and/or CDK4 inhibitors;
(v) antiangiogenic agents such as, but not limited to, those which inhibit the effects of vascular endothelial growth factor, [for example the anti-vascular endothelial cell growth factor antibody bevacizumab (Avastin™) and for example, a VEGF receptor tyrosine kinase inhibitor such as vandetanib (ZD6474), vatalanib (PTK787), sunitinib (SU11248), axitinib (AG-013736) and pazopanib (GW 786034).
(vi) vascular damaging agents such as, but not limited to, Combretastatin A4 and compounds disclosed in International Patent Applications WO 99/02166, WO 00/40529, WO 00/41669, WO 01/92224, WO 02/04434 and WO 02/08213;
(vii) an endothelin receptor antagonist, for example zibotentan (ZD4054) or atrasentan;
(viii) antisense therapies, such as, but not limited to, those directed to targets listed above, such as ISIS 2503, an anti-ras antisense;
(ix) immunotherapy approaches, including for example cancer vaccines, antibody, viral (oncolytic viruses) and small molecule or cell therapy approaches to increase the immunogenicity of patient tumour cells and/or facilitate a cell mediated anti-tumour response. Such therapies could include, but are not limited to, immune checkpoint inhibitors (e.g. CTLA4, LAG3, PD1, PD-L1, TIM-3 and/or TIGIT inhibitors), OX40 agonists, cGAS-STING agonists, A2a receptor antagonists, PI3 kinase inhibitors, TLR7/8 agonists, IDO inhibitors, immune stimulators (e.g. 4-1BB, OX40, cGAS-STING, CD27, CD40, and DR3 that enhance anti-tumour immunity), Arginase inhibitors, BTK inhibitors and Bromodomain inhibitors; transduction with microbial vectors of cancer antigens, direct transduction of cancer antigens into antigen presenting cells, treatment with immune cells specific for cancer antigens (e.g. CAR-T), treatment with antibodies, antibody fragments and antibody drug conjugates that enable the immune system to recognise tumour cells.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

According to this aspect of the invention there is provided a combination for use in the treatment of a cancer (for example a cancer involving a solid tumour) comprising a compound of the invention as defined hereinbefore, or a pharmaceutically acceptable salt or solvate thereof, and an anti-tumour agent. The compound of the invention is used combination with a source of M and/or Q, as required.

According to this aspect of the invention there is provided a combination for use in the treatment of a proliferative condition, such as cancer (for example a cancer involving a solid tumour), comprising a compound of the invention as defined hereinbefore, or a pharmaceutically acceptable salt or solvate thereof, and any one of the anti-tumour agents listed herein above. The compound of the invention is used combination with a source of M and/or Q, as required.

In a further aspect of the invention there is provided a compound of the invention or a pharmaceutically acceptable salt or solvate thereof, for use in the treatment of cancer in combination with another anti-tumour agent, optionally selected from one listed herein above. The compound of the invention is used combination with a source of M and/or Q, as required.

In a further aspect of the invention there is provided a compound of the invention or a pharmaceutically acceptable salt or solvate thereof, for use in the treatment of cancer in combination with a tyrosine kinase inhibitor, optionally selected from one listed herein above. The compound of the invention is used combination with a source of M and/or Q, as required.

Components used in combination with one another (e.g. the compounds of Formula I, II or III, sources of M and Q, anti-tumour agents and tyrosine kinase inhibitors) may be administered simultaneously, separately or sequentially. In one aspect of the invention in combination with refers to simultaneous administration. In another aspect of the invention in combination with refers to separate administration. In a further aspect of the invention in combination with refers to sequential administration. Where the administration is sequential or separate, the delay in administering the second (and any subsequent) component should not be such as to lose the beneficial effect of the combination.

According to a further aspect of the invention there is provided a pharmaceutical composition which comprises a compound of the invention, or a pharmaceutically acceptable salt or solvate thereof, in combination with an anti-tumour agent (optionally selected from one listed herein above) and/or a tyrosine kinase inhibitor, in association with a pharmaceutically acceptable diluent or carrier.

### PARTICULAR ASPECTS AND EMBODIMENTS

The following numbered statements 1-118 are not claims, but instead serve to define particular aspects and embodiments of the claimed invention.
1. A compound having a structure according to Formula I shown below, or a pharmaceutically acceptable salt, hydrate or solvate thereof:

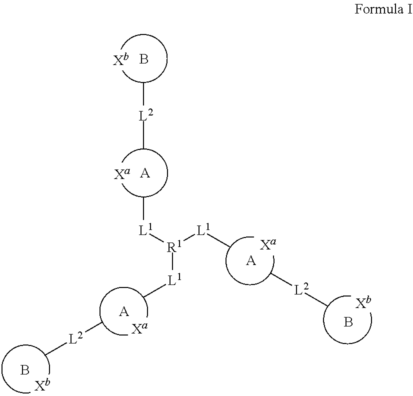

wherein
R¹ is selected from the group consisting of N, CR², aryl, heteroaryl, carbocyclyl and heterocyclyl, where any aryl, heteroaryl, carbocyclyl or heterocyclyl in R¹ is optionally substituted with one or more R³;
each R³ is independently selected from the group consisting of hydroxy, cyano, halogen, (1-4C)alkyl, (1-4C)haloalkyl, (2-4C)alkenyl, (2-4C)alkynyl, aryl, aryl(1-3C)alkyl, heteroaryl, heteroaryl(1-3C)alkyl, carbocyclyl, carbocyclyl(1-3C)alkyl, heterocyclyl, heterocyclyl(1-3C)alkyl, —OR^{3a}, —NR^{3a}R^{3b}, —C(O)—R^{3a}, —C(O)—OR^{3a}, —O—C(O)—R^{3a}, —C(O)—NR^{3a}R^{3b}, —N(R^{3a})C(O)—R^{3b} and —S(O)₀₋₂R^{3a}, where any (1-4C)alkyl, (1-4C)haloalkyl, (2-4C)alkenyl, (2-4C)alkynyl, aryl, aryl(1-3C)alkyl, heteroaryl, heteroaryl(1-3C)alkyl, carbocyclyl, carbocyclyl(1-3C)alkyl, heterocyclyl or heterocyclyl(1-3C)alkyl in R³ is optionally substituted with one or more R^{3c};
R^{3a} and R^{3b} are each independently selected from the group consisting of hydrogen, (1-3C)alkyl and (1-3C)haloalkyl;
each R^{3c} is independently selected from the group consisting of hydroxy, halogen, cyano, amino, (1-3C)alkyl, (1-3C)alkoxy and (1-3C)haloalkyl;
R² is selected from the group consisting of hydrogen, hydroxy, cyano, halogen, (1-4C)alkyl, (1-4C)haloalkyl, (2-4C)alkenyl, (2-4C)alkynyl, aryl, aryl(1-3C)alkyl, heteroaryl, heteroaryl(1-3C)alkyl, carbocyclyl, carbocyclyl(1-3C)alkyl, heterocyclyl, heterocyclyl(1-3C)alkyl, —OR^{2a}, —NR^{2a}R^{2b}, —C(O)—R^{2a}, —C(O)—OR^{2a}, —O—C(O)—R^{2a}, —C(O)—NR^{2a}R^{2b}, —N(R^{2a})C(O)—R^{2b} and —S(O)₀₋₂R^{9a}, where any (1-4C)alkyl, (1-4C)haloalkyl, (2-4C)alkenyl, (2-4C)alkynyl, aryl, aryl(1-3C)alkyl, heteroaryl, heteroaryl(1-3C)alkyl, carbocyclyl, carbocyclyl(1-3C)alkyl, heterocyclyl or heterocyclyl(1-3C)alkyl in R² is optionally substituted with one or more R^{2c};
R^{2a} and R^{2b} are each independently selected from the group consisting of hydrogen, (1-3C)alkyl and (1-3C)haloalkyl;
each R^{2c} is independently selected from the group consisting of hydroxy, halogen, cyano, amino, (1-3C)alkyl, (1-3C)alkoxy and (1-3C)haloalkyl;
each L¹ is a group:

—(W)ₙ—(X)ₘ—(Y)ₒ—(Z)ₚ—

in which
n and o are each independently 0, 1 or 2, and m and p are each independently 0 or 1, with the provisos that when m and p are both 1, o is not 0;
each W is selected from the group consisting of (1-3C)alkylene, (2-3C)alkenylene, (2-3C)alkynylene, arylene, heteroarylene, carbocyclylene and heterocyclylene, where any (1-3C)alkylene, (2-3C)alkenylene, (2-3C)alkynylene, arylene, heteroarylene, carbocyclylene or heterocyclylene in W is optionally substituted with one or more W^{a}, where each W^{a} is independently selected from the group consisting of hydroxy, cyano, halogen, amino, (1-2)alkoxy and (1-2C)haloalkyl;
X is selected from the group consisting of —O—, —C(O)—, —C(O)—O—, —O—C(O)—, —S(O)₀₋₂—, —C(O)—N(R^{x})—, —N(R^{x})—C(O)—, —NR^{x}—, —N(R^{x})—C(O)—NR^{x}—, —SO₂N(R^{x})—, and —N(R^{x})SO₂, where each R^{x} is independently selected from the group consisting of hydrogen, hydroxy, cyano, (1-4C)alkyl, (2-4C)alkenyl and (2-4C)alkynyl;
each Y is selected from the group consisting of (1-3C)alkylene, (2-3C)alkenylene, (2-3C)alkynylene, arylene, heteroarylene, carbocyclylene and heterocyclylene, where any (1-3C)alkylene, (2-3C)alkenylene, (2-3C)alkynylene, arylene, heteroarylene, carbocyclylene or heterocyclylene in Y is optionally substituted with one or more Y^{a}, where each Y^{a} is independently selected from the group consisting of hydroxy, cyano, halogen, amino, (1-2)alkoxy and (1-2C)haloalkyl;
Z is selected from the group consisting of —O—, —C(O)—, —C(O)—O—, —O—C(O)—, —S(O)₀₋₂—, —C(O)—N(R^{z})—, —N(R^{z})—C(O)—, —NR^{z}—, —N(R^{z})—C(O)—NR^{z}—, —SO₂N(R^{z})—, and —N(R^{z})SO₂, where each R^{z} is independently selected from the group consisting of hydrogen, hydroxy, cyano, (1-4C)alkyl, (2-4C)alkenyl and (2-4C)alkynyl;
X^{a} is a ring heteroatom located within ring A and is selected from N and O;
each ring A is a monocyclic heteroaryl, bicyclic heteroaryl, monocyclic heterocycle or bicyclic heterocycle, any one of which is optionally substituted with one or more R⁴, where each R⁴ is independently selected from the group consisting of hydroxy, cyano, halogen, (1-6C)alkyl, (1-6C)haloalkyl, (2-6C)alkenyl, (2-6C)alkynyl, aryl, aryl(1-3C)alkyl, heteroaryl, heteroaryl(1-3C)alkyl, carbocyclyl, carbocyclyl(1-3C)alkyl, heterocyclyl, heterocyclyl(1-3C)alkyl, —R^{4a}—OR^{4b}, —R^{4a}—NR^{4b}R^{4c}, —R^{4a}—C(O)—R^{4b}, —R^{4a}—C(O)—OR^{4b}, —R^{4a}—O—C(O)—R^{4b}, —R^{4a}—C(O)—NR^{4b}R^{4c}, —R^{4a}. N(R^{4b})C(O)—R^{4c} and —R^{4a}—S(O)₀₋₂R^{4b}, where any (1-6C)alkyl, (1-6C)haloalkyl, (2-6C)alkenyl, (2-6C)alkynyl, aryl, aryl(1-3C)alkyl, heteroaryl, heteroaryl(1-3C)alkyl, carbocyclyl, carbocyclyl(1-3C)alkyl, heterocyclyl or heterocyclyl(1-3C)alkyl in R⁴ is optionally substituted with one or more R^{4d};
R^{4a} is absent or is (1-3C)alkylene that is optionally substituted with one or substituents selected from group consisting of hydroxy, halo and amino;
R^{4b} and R^{4c} are each independently selected from the group consisting of hydrogen, (1-3C)alkyl and (1-3C)haloalkyl;
each R^{4d} is independently selected from the group consisting of hydroxy, halogen, cyano, amino, (1-3C)alkyl, (1-3C)alkoxy and (1-3C)haloalkyl;
X^{b} is a ring heteroatom located within ring B and is selected from N and O
each ring B is a monocyclic heteroaryl, bicyclic heteroaryl, monocyclic heterocycle or bicyclic heterocycle, any one of which is optionally substituted with one or more R⁵, where each R⁵ is independently selected from the group consisting of hydroxy, cyano, halogen, (1-6C)alkyl, (1-6C)haloalkyl, (2-6C)alkenyl, (2-6C)alkynyl, aryl, aryl(1-3C)alkyl, heteroaryl, heteroaryl(1-3C)alkyl, carbocyclyl, carbocyclyl(1-3C)alkyl, heterocyclyl, heterocyclyl(1-3C)alkyl, —R^{5a}—OR^{5b}, —R^{5a}—NR^{5b}R^{5c}, —R^{5a}—C(O)—R^{5b}, —R^{5a}—C(O)—OR^{5b}, —R^{5a}—O—C(O)—R^{5b}, —R^{5a}—C(O)—NR^{5b}R^{5c}, —R^{5a}. N(R^{5b})C(O)—R^{5c} and —R^{5a}—S(O)₀₋₂R^{5b}, where any (1-6C)alkyl, (1-6C)haloalkyl, (2-6C)alkenyl, (2-6C)alkynyl, aryl, aryl(1-3C)alkyl, heteroaryl, heteroaryl(1-3C)alkyl, carbocyclyl, carbocyclyl(1-3C)alkyl, heterocyclyl or heterocyclyl(1-3C)alkyl in R⁵ is optionally substituted with one or more R^{5d};
R^{5a} is absent or is (1-3C)alkylene that is optionally substituted with one or substituents selected from group consisting of hydroxy, halo and amino;
R^{5b} and R^{5c} are each independently selected from the group consisting of hydrogen, (1-5C)alkyl (e.g. (1-3C)alkyl) and (1-3C)haloalkyl;
each R^{5d} is independently selected from the group consisting of hydroxy, halogen, cyano, amino, (1-3C)alkyl, (1-3C)alkoxy and (1-3C)haloalkyl;
each L² is selected from the group consisting of absent (in which case ring A is bonded directly to ring B), (1-2C)alkylene, ethenylene and ethynylene, where any (1-2C)alkylene, ethenylene and ethynylene in L² is optionally substituted with one or more substituents selected form the group consisting of hydroxy, halogen, cyano, amino, (1-3C)alkyl, (1-3C)alkoxy and (1-3C)haloalkyl;
with the proviso that the compound of Formula I is not the following Compound Ia, or a pharmaceutically acceptable salt, hydrate or solvate thereof:

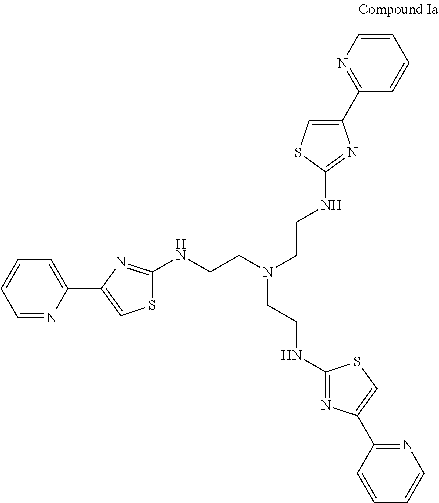 [SMILES: c1ccc(-c2csc(NCCN(CCNc3nc(-c4ccccn4)cs3)CCNc3nc(-c4ccccn4)cs3)n2)nc1]

2. The compound, pharmaceutically acceptable salt, hydrate or solvate of statement 1, wherein each ring A is a 5-7 membered monocyclic heteroaryl containing 1, 2 or 3 ring heteroatoms in total independently selected from N, O and S, or a 5-7 membered monocyclic heterocycle containing 1, 2 or 3 ring heteroatoms in total independently selected from N, O and S, wherein each ring A is optionally substituted with one or more R⁴.
3. The compound, pharmaceutically acceptable salt, hydrate or solvate of statement 1 or 2, wherein each ring A is a 5-6 membered monocyclic heteroaryl containing 1, 2 or 3 ring heteroatoms in total independently selected from N, O and S, or a 5-6 membered monocyclic heterocycle containing 1, 2 or 3 ring heteroatoms in total independently selected from N, O and S, wherein each ring A is optionally substituted with one or more R⁴.
4. The compound, pharmaceutically acceptable salt, hydrate or solvate of statement 1, 2 or 3, wherein X^{a} is N and ring A contains 0, 1 or 2 further ring heteroatoms selected from N, O and S.
5. The compound, pharmaceutically acceptable salt, hydrate or solvate of statement 1, 2 or 3, wherein X^{a} is N and ring A contains 0 or 1 further ring heteroatoms selected from N, O and S.
6. The compound, pharmaceutically acceptable salt, hydrate or solvate of any preceding statement, wherein X^{a} is located immediately adjacent the carbon atom bonded to L¹.
7. The compound, pharmaceutically acceptable salt, hydrate or solvate of any preceding statement, wherein each ring A is group:

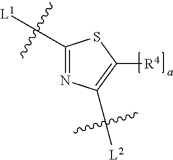

wherein a is 0 or 1.
8. The compound, pharmaceutically acceptable salt, hydrate or solvate of any preceding statement, wherein each R⁴ is independently selected from the group consisting of hydroxy, halogen, (1-6C)alkyl, (1-6C)haloalkyl, (2-6C)alkenyl, phenyl, phenyl(1-3C)alkyl, 5-6 membered heteroaryl, 5-6 membered heteroaryl(1-3C)alkyl, —R^{4a}—OR^{4b}, —R^{4a}—NR^{4b}R^{4c}, —R^{4a}—C(O)—R^{4b}, —R^{4a}. C(O)—OR^{4b}, —R^{4a}—O—C(O)—R^{4b}, —R^{4a}—C(O)—NR^{4b}R^{4c}, —R^{4a}—N(R^{4b})C(O)—R^{4c} and —R^{4a}—S(O)₀₋₂R^{4b}, where any (1-6C)alkyl, (1-6C)haloalkyl, (2-6C)alkenyl, phenyl, phenyl(1-3C)alkyl, 5-6 membered heteroaryl or 5-6 membered heteroaryl(1-3C)alkyl in R⁴ is optionally substituted with one or more R^{4d}.
9. The compound, pharmaceutically acceptable salt, hydrate or solvate of any preceding statement, wherein each R⁴ is independently selected from the group consisting of hydroxy, halogen, (1-3C)alkyl, (1-3C)haloalkyl, (2-3C)alkenyl, phenyl and —R^{4a}—OR^{4b}, where any (1-3C)alkyl, (1-3C)haloalkyl, (2-3C)alkenyl or phenyl in R⁴ is optionally substituted with one or more R^{4d}.
10. The compound, pharmaceutically acceptable salt, hydrate or solvate of any preceding statement, wherein each R^{4a} is absent or methylene.
11. The compound, pharmaceutically acceptable salt, hydrate or solvate of any preceding statement, wherein R^{4b} and R^{4c} are each independently selected from the group consisting of hydrogen, methyl and ethyl.
12. The compound, pharmaceutically acceptable salt, hydrate or solvate of any preceding statement, wherein each R^{4d} is independently selected from the group consisting of hydroxy, halogen, amino, (1-2C)alkyl, (1-2C)alkoxy and (1-2C)haloalkyl.
13. The compound, pharmaceutically acceptable salt, hydrate or solvate of any preceding statement, wherein each ring B is:
   i) a 5-7 membered monocyclic heterocycle containing 1, 2 or 3 ring heteroatoms in total that are independently selected from N, O and S;
   ii) a 5-7 membered monocyclic heteroaryl containing 1, 2 or 3 ring heteroatoms in total that are independently selected from N, O and S;
   iii) a 8-10 membered bicyclic heterocycle containing 1, 2, 3 or 4 ring heteroatoms in total that are independently selected from N, O and S; or
   iv) a 8-10 membered bicyclic heteroaryl containing 1, 2, 3 or 4 ring heteroatoms in total that are independently selected from N, O and S,
   wherein any ring in B is optionally substituted with one or more R⁵.
14. The compound, pharmaceutically acceptable salt, hydrate or solvate of any preceding statement, wherein each ring B is:
   i) a 5-6 membered monocyclic heterocycle containing 1, 2 or 3 ring heteroatoms in total that are independently selected from N, O and S;
   ii) a 5-6 membered monocyclic heteroaryl containing 1, 2 or 3 ring heteroatoms in total that are independently selected from N, O and S;
   iii) a 9-10 membered bicyclic heterocycle containing 1, 2 or 3 ring heteroatoms in total that are independently selected from N, O and S; or
   iv) a 9-10 membered bicyclic heteroaryl containing 1, 2 or 3 ring heteroatoms in total that are independently selected from N, O and S,
   wherein any ring in B is optionally substituted with one or more R⁵.
15. The compound, pharmaceutically acceptable salt, hydrate or solvate of any preceding statement, wherein X^{b} is N and ring A contains 0, 1 or 2 further ring heteroatoms selected from N, O and S.
16. The compound, pharmaceutically acceptable salt, hydrate or solvate of any preceding statement, wherein X^{b} is N and ring A contains 0 or 1 further ring heteroatoms selected from N, O and S.
17. The compound, pharmaceutically acceptable salt, hydrate or solvate of any preceding statement, wherein X^{b} is located immediately adjacent the carbon atom bonded to L².
18. The compound, pharmaceutically acceptable salt, hydrate or solvate of any preceding statement, wherein each ring B is any of the following:

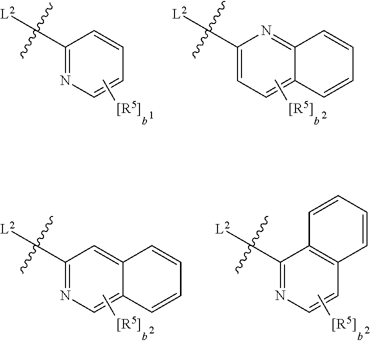

wherein:
b¹ is 0, 1, 2 or 3, and
b² is 0, 1, 2, 3 or 4.
19. The compound, pharmaceutically acceptable salt, hydrate or solvate of any preceding statement, wherein each ring B is any of the following:

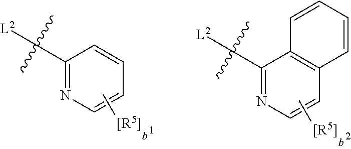

wherein:
b¹ is 0, 1 or 2, and
b² is 0, 1, 2 or 3.
20. The compound, pharmaceutically acceptable salt, hydrate or solvate of any preceding statement, wherein each R⁵ is independently selected from the group consisting of hydroxy, halogen, (1-6C)alkyl, (1-6C)haloalkyl, (2-6C)alkenyl, phenyl, phenyl(1-3C)alkyl, 5-6 membered heteroaryl, 5-6 membered heteroaryl(1-3C)alkyl, —R^{5a}—OR^{5b}, —R^{5a}—NR^{5b}R^{5c}, —R^{5a}—C(O)—R^{5b}, —R^{5a}—C(O)—OR^{5b}, —R^{5a}—O—C(O)—R^{5b}, —R^{5a}—C(O)—NR^{5b}R^{5c}, —R^{5a}—N(R^{5b})C(O)—R^{5c} and —R^{5a}—S(O)₀₋₂R^{5b}, where any (1-6C)alkyl, (1-6C)haloalkyl, (2-6C)alkenyl, phenyl, phenyl(1-3C)alkyl, 5-6 membered heteroaryl or 5-6 membered heteroaryl(1-3C)alkyl in R⁵ is optionally substituted with one or more R^{5d}.
21. The compound, pharmaceutically acceptable salt, hydrate or solvate of any preceding statement, wherein each R⁵ is independently —R^{5a}—O—C(O)—R^{5b}.
22. The compound, pharmaceutically acceptable salt, hydrate or solvate of any preceding statement, wherein each R^{5a} is absent or methylene.
23. The compound, pharmaceutically acceptable salt, hydrate or solvate of any preceding statement, wherein R^{5b} and R^{5c} are each independently selected from the group consisting of hydrogen, methyl, ethyl and pentyl (e.g. hydrogen, methyl and ethyl).
24. The compound, pharmaceutically acceptable salt, hydrate or solvate of any preceding statement, wherein each R^{5d} is independently selected from the group consisting of hydroxy, halogen, amino, (1-2C)alkyl, (1-2C)alkoxy and (1-2C)haloalkyl.
25. The compound, pharmaceutically acceptable salt, hydrate or solvate of any preceding statement, wherein each W is selected from the group consisting of (1-3C)alkylene, phenylene, 5-6 membered heteroarylene, 5-6 membered carbocyclylene and 5-6 membered heterocyclylene, where any (1-3C)alkylene, phenylene, 5-6 membered heteroarylene, 5-6 membered carbocyclylene or 5-6 membered heterocyclylene in W is optionally substituted with one or more W^{a}.
26. The compound, pharmaceutically acceptable salt, hydrate or solvate of any preceding statement, wherein each W is selected from the group consisting of (1-3C)alkylene or phenylene, where any (1-3C)alkylene or phenylene in W is optionally substituted with one or more W^{a}.
27. The compound, pharmaceutically acceptable salt, hydrate or solvate of any preceding statement, wherein each W^{a} is independently selected from the group consisting of hydroxy, halogen, (1-2)alkoxy and (1-2C)haloalkyl.
28. The compound, pharmaceutically acceptable salt, hydrate or solvate of any preceding statement, wherein X is selected from the group consisting of —O—, —C(O)—, —C(O)—O—, —O—C(O)—, —C(O)—N(R^{x})—, —N(R^{x})—C(O)— and —NR^{x}—.
29. The compound, pharmaceutically acceptable salt, hydrate or solvate of any preceding statement, wherein X is —NR^{x}—.
30. The compound, pharmaceutically acceptable salt, hydrate or solvate of any preceding statement, wherein each R^{x} is independently selected from the group consisting of hydrogen, hydroxy and (1-4C)alkyl.
31. The compound, pharmaceutically acceptable salt, hydrate or solvate of any preceding statement, wherein each R^{x} is hydrogen.
32. The compound, pharmaceutically acceptable salt, hydrate or solvate of any preceding statement, wherein each Y is selected from the group consisting of (1-3C)alkylene, phenylene, 5-6 membered heteroarylene, 5-6 membered carbocyclylene and 5-6 membered heterocyclylene, where any (1-3C)alkylene, phenylene, 5-6 membered heteroarylene, 5-6 membered carbocyclylene or 5-6 membered heterocyclylene in Y is optionally substituted with one or more Y^{a}.
33. The compound, pharmaceutically acceptable salt, hydrate or solvate of any preceding statement, wherein each Y is selected from the group consisting of (1-3C)alkylene or phenylene, where any (1-3C)alkylene or phenylene in Y is optionally substituted with one or more Y^{a}.
34. The compound, pharmaceutically acceptable salt, hydrate or solvate of any preceding statement, wherein each Y^{a} is independently selected from the group consisting of hydroxy, halogen, (1-2)alkoxy and (1-2C)haloalkyl.
35. The compound, pharmaceutically acceptable salt, hydrate or solvate of any preceding statement, wherein Z is selected from the group consisting of —O—, —C(O)—, —C(O)—O—, —O—C(O)—, —C(O)—N(R^{z})—, —N(R^{z})—C(O)— and —NR^{z}—.
36. The compound, pharmaceutically acceptable salt, hydrate or solvate of any preceding statement, wherein Z is —NR^{z}—.
37. The compound, pharmaceutically acceptable salt, hydrate or solvate of any preceding statement, wherein each R^{z} is independently selected from the group consisting of hydrogen, hydroxy and (1-4C)alkyl.
38. The compound, pharmaceutically acceptable salt, hydrate or solvate of any preceding statement, wherein each R^{z} is hydrogen.
39. The compound, pharmaceutically acceptable salt, hydrate or solvate of any preceding statement, wherein:
   n is 0 or 1;
   m is 0;
   o is 0 or 1; and
   p is 1.
40. The compound, pharmaceutically acceptable salt, hydrate or solvate of any preceding statement, wherein:
   n is 0 or 1 and W is selected from the group consisting of (1-3C)alkylene or phenylene, where any (1-3C)alkylene or phenylene in W is optionally substituted with one or more W^{a}, where each W^{a} is independently selected from the group consisting of hydroxy, halogen, (1-2)alkoxy and (1-2C)haloalkyl;
   m is 0;
   o is 0 or 1 and Y is selected from the group consisting of (1-3C)alkylene or phenylene, where any (1-3C)alkylene or phenylene in Y is optionally substituted with one or more Y^{a}, where each Y^{a} is independently selected from the group consisting of hydroxy, halogen, (1-2)alkoxy and (1-2C)haloalkyl; and
   p is 1 and Z is —NR^{z}—, where R^{z} is hydrogen.
41. The compound, pharmaceutically acceptable salt, hydrate or solvate of any preceding statement, wherein L¹ has a structure according to any one of the following:

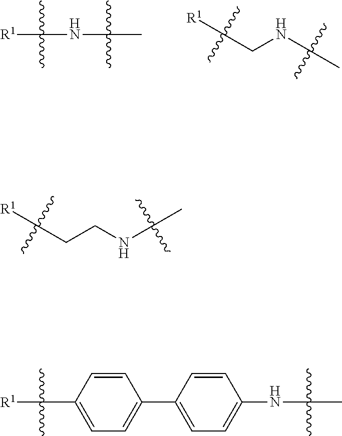

42. The compound, pharmaceutically acceptable salt, hydrate or solvate of any preceding statement, wherein L² is selected from the group consisting of absent and (1-2C)alkylene, where any (1-2C)alkylene in L² is optionally substituted with one or more substituents selected form the group consisting of hydroxy, halogen, amino, (1-2C)alkyl, (1-2C)alkoxy and (1-2C)haloalkyl.
43. The compound, pharmaceutically acceptable salt, hydrate or solvate of any preceding statement, wherein L² is selected from the group consisting of absent and (1-2C)alkylene, where any (1-2C)alkylene in L² is optionally substituted with one or more substituents selected form the group consisting of hydroxy, halogen and (1-2C)haloalkyl.
44. The compound, pharmaceutically acceptable salt, hydrate or solvate of any preceding statement, wherein L² is selected from the group consisting of absent, methylene and ethylene.
45. The compound, pharmaceutically acceptable salt, hydrate or solvate of any preceding statement, wherein L² is absent.
46. The compound, pharmaceutically acceptable salt, hydrate or solvate of any preceding statement, wherein R¹ is selected from the group consisting of N, CR², phenyl, 6 membered heteroaryl, 6 membered carbocyclyl and 6 membered heterocyclyl, where any phenyl, 6 membered heteroaryl, 6 membered carbocyclyl or 6 membered heterocyclyl in R¹ is optionally substituted with one or more R³.
47. The compound, pharmaceutically acceptable salt, hydrate or solvate of any preceding statement, wherein R¹ is selected from the group consisting of N, CR², phenyl and cyclohexyl, where any phenyl or cyclohexyl in R¹ is optionally substituted with one or more R³.
48. The compound, pharmaceutically acceptable salt, hydrate or solvate of any preceding statement, wherein each R³ is independently selected from the group consisting of hydroxy, halogen, (1-4C)alkyl, (1-4C)haloalkyl and —OR^{3a}, where any (1-4C)alkyl or (1-4C)haloalkyl in R³ is optionally substituted with one or more R^{3c}
49. The compound, pharmaceutically acceptable salt, hydrate or solvate of any preceding statement, wherein each R^{3c} is independently selected from the group consisting of hydroxy, halogen, amino, (1-2C)alkoxy and (1-2C)haloalkyl.
50. The compound, pharmaceutically acceptable salt, hydrate or solvate of any preceding statement, wherein R² is selected from the group consisting of hydrogen, hydroxy, halogen, (1-4C)alkyl, (1-4C)haloalkyl and —OR^{2a}, where any (1-4C)alkyl or (1-4C)haloalkyl in R² is optionally substituted with one or more R^{2c}.
51. The compound, pharmaceutically acceptable salt, hydrate or solvate of any preceding statement, wherein R² is selected from the group consisting of hydrogen, and (1-3C)alkyl, where any (1-4C)alkyl in R² is optionally substituted with one or more R^{2c}.
52. The compound, pharmaceutically acceptable salt, hydrate or solvate of any preceding statement, wherein R² is selected from the group consisting of hydrogen, methyl or ethyl.
53. The compound, pharmaceutically acceptable salt, hydrate or solvate of any preceding statement, wherein each R^{2c} is independently selected from the group consisting of hydroxy, halogen, amino, (1-2C)alkoxy and (1-2C)haloalkyl
54. The compound, pharmaceutically acceptable salt, hydrate or solvate of any preceding statement, wherein R¹ has a structure according to any one of the following:

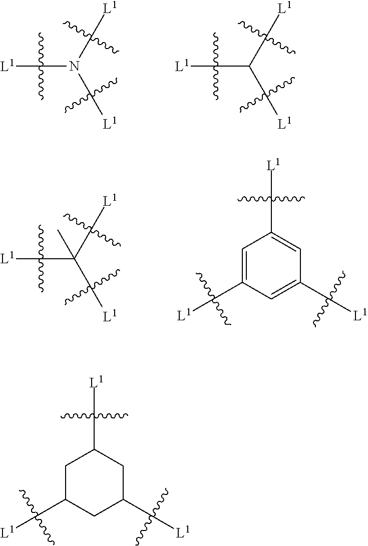

55. The compound, pharmaceutically acceptable salt, hydrate or solvate of any preceding statement, wherein R¹ has a structure according to the following:

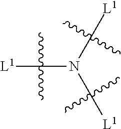

56. The compound, pharmaceutically acceptable salt, hydrate or solvate of any preceding statement, wherein the compound has a structure according to Formula I-I:

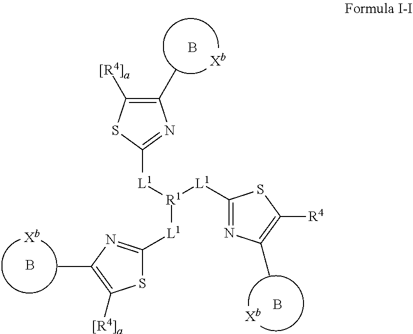

wherein R¹, L¹, R⁴, a, B, X^{b} and any associated subgroups are as defined in any preceding statement.
57. The compound, pharmaceutically acceptable salt, hydrate or solvate of any one of statements 1-55, wherein the compound has a structure according to Formula I-II:

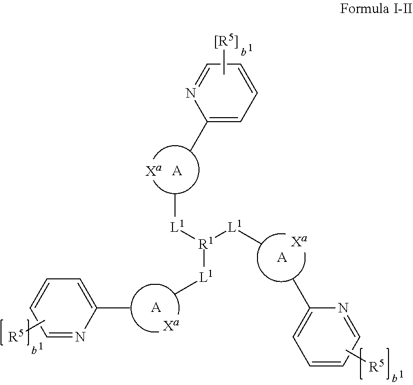

wherein
each R⁵ is as defined in any preceding statement, or two R⁵ groups located on adjacent carbon atoms are linked, such that when taken in combination with the atoms to which they are attached, they form a fused benzene ring that is optionally substituted with one or more groups R⁵; and
R¹, L¹, A, X^{a}, b¹ and any associated subgroups are as defined in any preceding statement.
58. The compound, pharmaceutically acceptable salt, hydrate or solvate of any one of statements 1-55, wherein the compound has a structure according to Formula I-III:

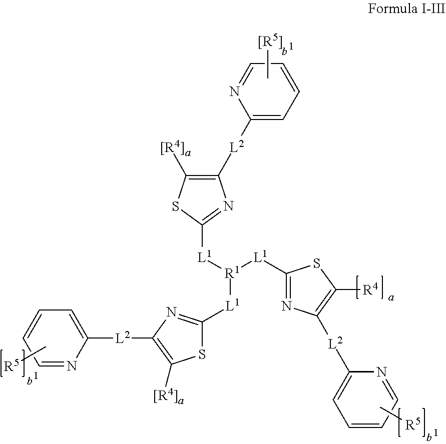

wherein
each R⁵ is as defined in any preceding statement, or two R⁵ groups located on adjacent carbon atoms are linked, such that when taken in combination with the atoms to which they are attached, they form a fused benzene ring that is optionally substituted with one or more groups R⁵; and
R¹, L¹, R⁴, a, L², b¹ and any associated subgroups are as defined in any preceding statement.
59. The compound, pharmaceutically acceptable salt, hydrate or solvate of any one of statements 1-55, wherein the compound has a structure according to Formula I-IV:

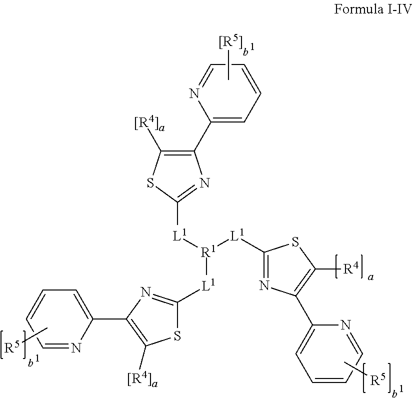

wherein
each R⁵ is as defined in any preceding statement, or two R⁵ groups located on adjacent carbon atoms are linked, such that when taken in combination with the atoms to which they are attached, they form a fused benzene ring that is optionally substituted with one or more groups R⁵; and
R¹, L¹, R⁴, a, b¹ and any associated subgroups are as defined in any preceding statement.
60. The compound, pharmaceutically acceptable salt, hydrate or solvate of any one of statements 1-55, wherein the compound has a structure according to Formula I-V:

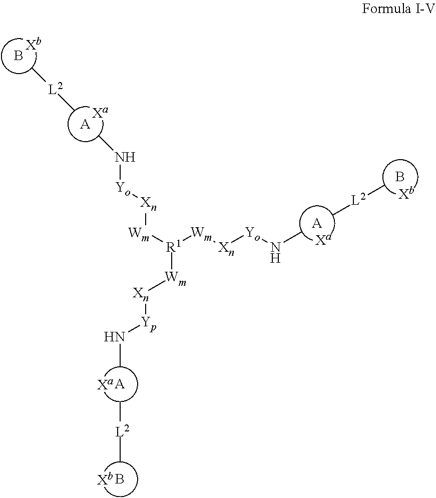

wherein R¹, W, X, Y, m, n, o, A, X^{a}, L², B, X^{b} and any associated subgroups are as defined in any preceding statement.
   61. The compound, pharmaceutically acceptable salt, hydrate or solvate of any one of statements 1-55, wherein the compound has a structure according to Formula I-VI:

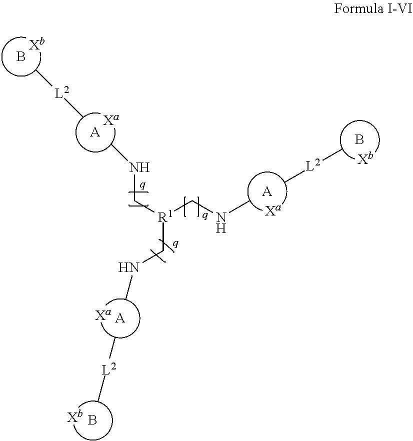

wherein q is 0, 1, 2 or 3; and
R¹, A, X^{a}, L², B, X^{b} and any associated subgroups are as defined in any preceding statement.
62. The compound, pharmaceutically acceptable salt, hydrate or solvate of any one of statements 1-55, wherein the compound has a structure according to Formula I-VII:

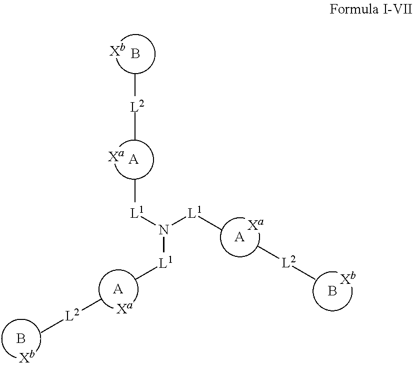

wherein L¹, A, X^{a}, L², B, X^{b} and any associated subgroups are as defined in any preceding statement.
63. The compound, pharmaceutically acceptable salt, hydrate or solvate of any one of statements 1-55, wherein the compound has a structure according to Formula I-VIII:

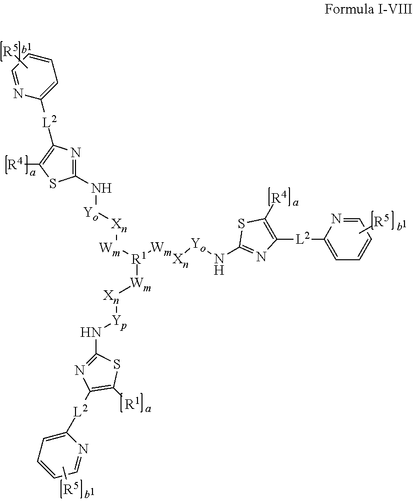

wherein
each R⁵ is as defined in any preceding statement, or two R⁵ groups located on adjacent carbon atoms are linked, such that when taken in combination with the atoms to which they are attached, they form a fused benzene ring that is optionally substituted with one or more groups R⁵; and
R¹, W, X, Y, m, n, o, R⁴, a, L², b¹ and any associated subgroups are as defined in any preceding statement.
64. The compound, pharmaceutically acceptable salt, hydrate or solvate of any one of statements 1-55, wherein the compound has a structure according to Formula I-IX:

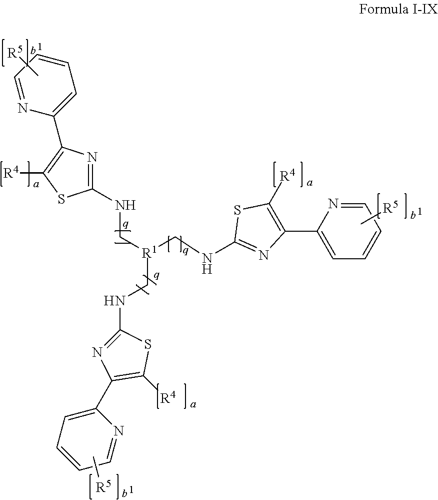

wherein
q is 0, 1, 2 or 3;
each R⁵ is as defined in any preceding statement, or two R⁵ groups located on adjacent carbon atoms are linked, such that when taken in combination with the atoms to which they are attached, they form a fused benzene ring that is optionally substituted with one or more groups R⁵; and
R¹, R⁴, a, b¹ and any associated subgroups are as defined in any preceding statement.
65. The compound, pharmaceutically acceptable salt, hydrate or solvate of any one of statements 1-55, wherein the compound has a structure according to Formula I-X:

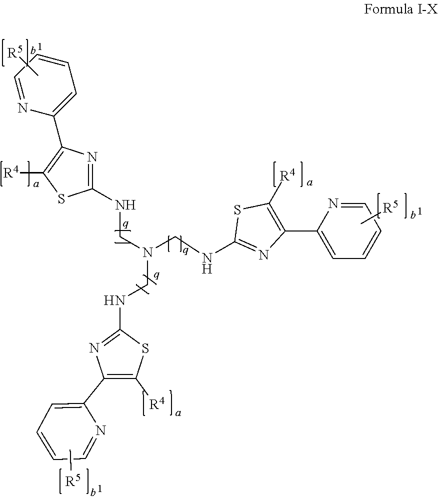

wherein
q is 0, 1, 2 or 3;
each R⁵ is as defined in any preceding statement, or two R⁵ groups located on adjacent carbon atoms are linked, such that when taken in combination with the atoms to which they are attached, they form a fused benzene ring that is optionally substituted with one or more groups R⁵; and
R⁴, a, L², b¹ and any associated subgroups are as defined in any preceding statement.
66. The compound, pharmaceutically acceptable salt, hydrate or solvate of statement 1, wherein the compound has a structure according to any one of the following:

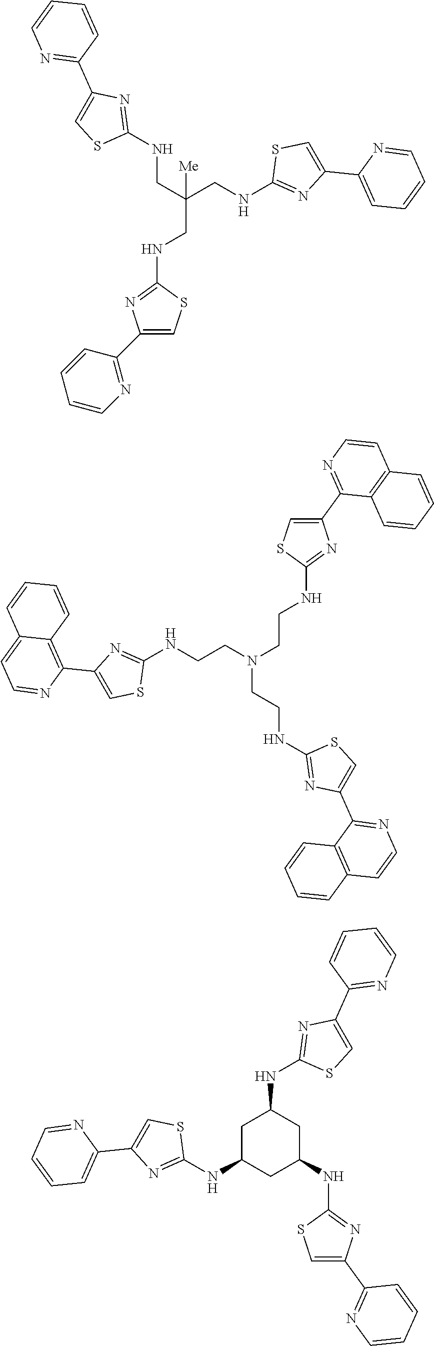

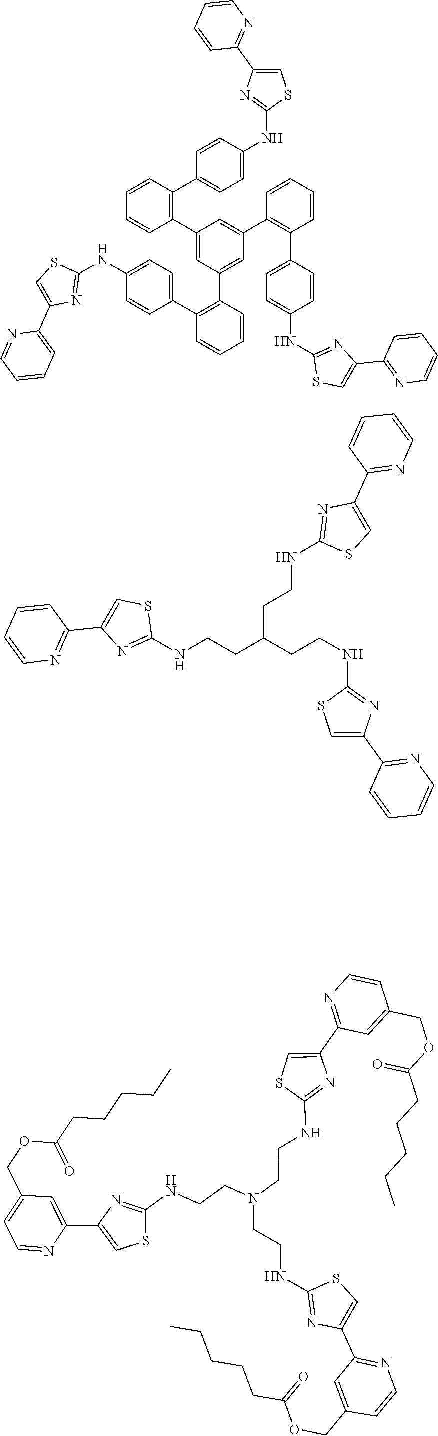

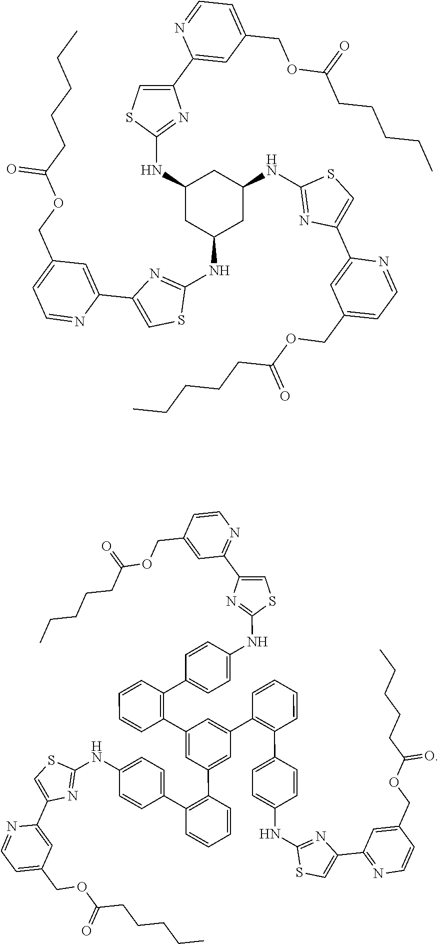 [SMILES: CCCCCC(=O)OCc1ccnc(-c2csc(N[C@H]3C[C@@H](Nc4nc(-c5cc(COC(=O)CCCCC)ccn5)cs4)C[C@@H](Nc4nc(-c5cc(COC(=O)CCCCC)ccn5)cs4)C3)n2)c1 ; CCCCCC(=O)OCc1ccnc(-c2csc(Nc3ccc(-c4ccccc4-c4cc(-c5ccccc5-c5ccc(Nc6nc(-c7cc(COC(=O)CCCCC)ccn7)cs6)cc5)cc(-c5ccccc5-c5ccc(Nc6nc(-c7cc(COC(=O)CCCCC)ccn7)cs6)cc5)c4)cc3)n2)c1]

67. A compound having a structure according to Formula II shown below, or a pharmaceutically acceptable salt, hydrate or solvate thereof:

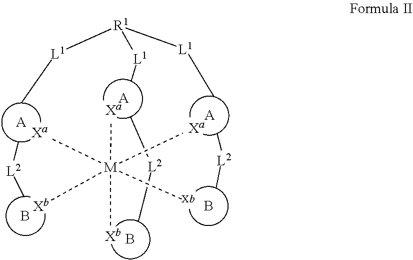

wherein
M is selected from the group consisting of Zn²⁺, Mn²⁺, Cu²⁺, Fe²⁺, CO²⁺ and Ni²⁺; and
R¹, L¹, A, X^{a}, L², B, X^{b} and any associated subgroups are as defined in any one of statements 1 to 55.
68. The compound, pharmaceutically acceptable salt, hydrate or solvate of statement 67, wherein the compound is not Compound IIa shown below, or a pharmaceutically acceptable salt, hydrate or solvate thereof:

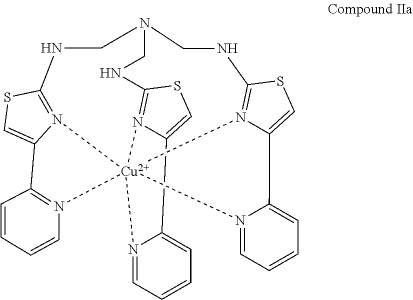

69. The compound, pharmaceutically acceptable salt, hydrate or solvate of statement 67, wherein M is selected from the group consisting of Zn²⁺, Mn²⁺, Fe²⁺, CO²⁺ and Ni²⁺.
70. The compound, pharmaceutically acceptable salt, hydrate or solvate of statement 67, wherein M is selected from the group consisting of Zn²⁺, Mn²⁺ and Fe²⁺.
71. The compound, pharmaceutically acceptable salt, hydrate or solvate of statement 67, wherein M is selected from the group consisting of Zn²⁺ and Mn²⁺.
72. The compound, pharmaceutically acceptable salt, hydrate or solvate of any one of statements 67 to 71, wherein the compound has a structure according to any one of the following:

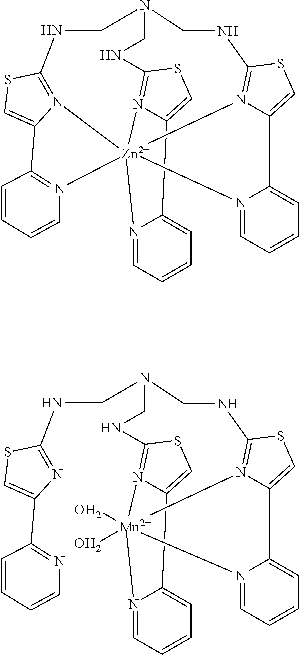

73. A compound having a structure according to Formula III shown below, or a pharmaceutically acceptable salt, hydrate or solvate thereof:

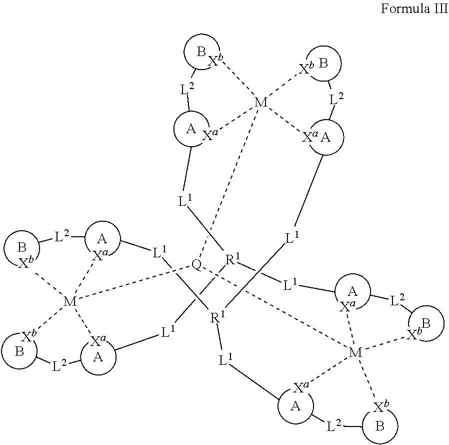

wherein
R¹, L¹, A, X^{a}, L², B, X^{b} and any associated subgroups are as defined in any one of statements 1 to 55;
M is selected from the group consisting of Zn²⁺, Mn²⁺, Cu²⁺, Fe²⁺, CO²⁺ and Ni²⁺;
Q is an anion selected from the group consisting of spherical monoanionic anions, trigonal planar anions, dianionic tetrahedral anions, trianionic tetrahedral anions, dianionic octahedral anions and trianionic octahedral anions;
with the proviso that the compound does not have a structure according to Formula IIIa:

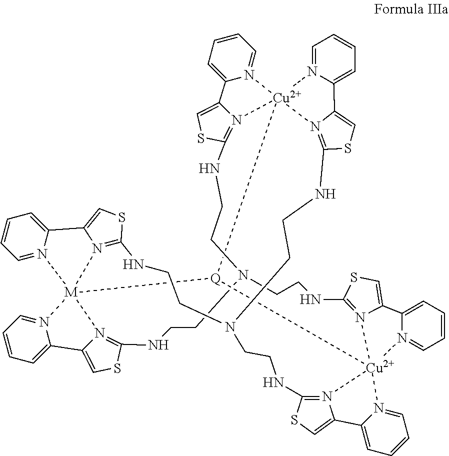

in which Q is Br⁻, I⁻, CO₃²⁻, SiF₆²⁻, IO₆³⁻, VO₄³⁻, WO₄²⁻, CrO₄²⁻, SO₄²⁻, HSO₄⁻, AsO₄³⁻, PO₄³⁻ or HPO₄²⁻.
74. The compound, pharmaceutically acceptable salt, hydrate or solvate of statement 73, wherein M is selected from the group consisting of Zn²⁺, Mn²⁺, Fe²⁺, CO²⁺ and Ni²⁺.
75. The compound, pharmaceutically acceptable salt, hydrate or solvate of statement 73, wherein M is selected from the group consisting of Zn²⁺, Mn²⁺ and Fe²⁺.
76. The compound, pharmaceutically acceptable salt, hydrate or solvate of statement 73, wherein M is selected from the group consisting of Zn²⁺ and Mn²⁺.
77. The compound, pharmaceutically acceptable salt, hydrate or solvate of any one of statements 73 to 76, wherein Q is sulfate (SO₄²⁻), phosphate (PO₄³⁻) or organophosphate (such as monophenylphosphate).
78. The compound, pharmaceutically acceptable salt, hydrate or solvate of any one of statements 73 to 77, wherein the compound has a structure according to any one of the following:

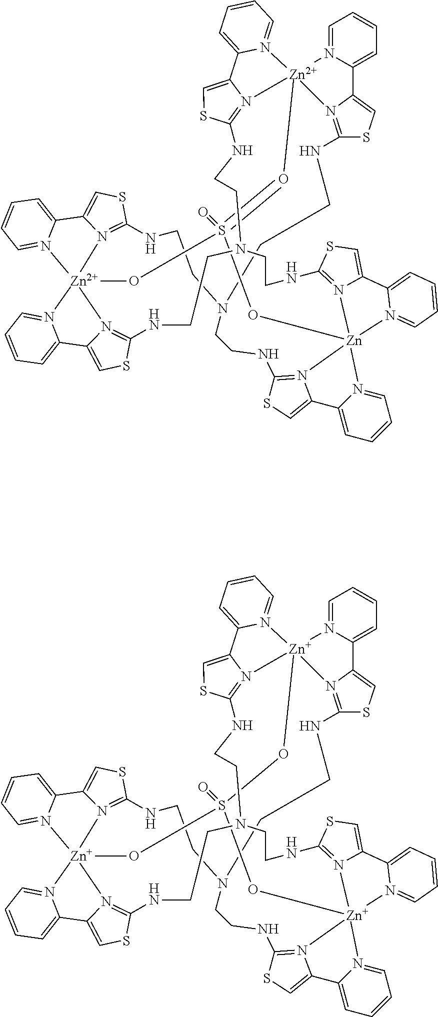

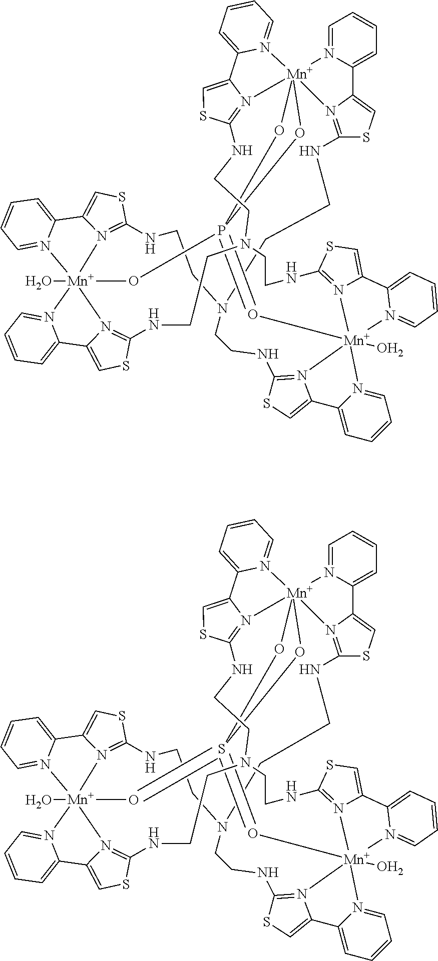

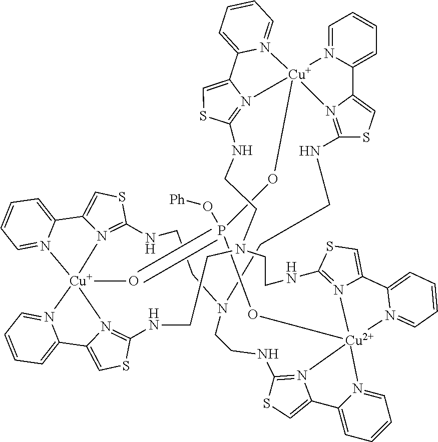

79. A compound of Formula I shown below, or a pharmaceutically acceptable salt, hydrate or solvate thereof, in combination with a source of M, for use as a medicament:

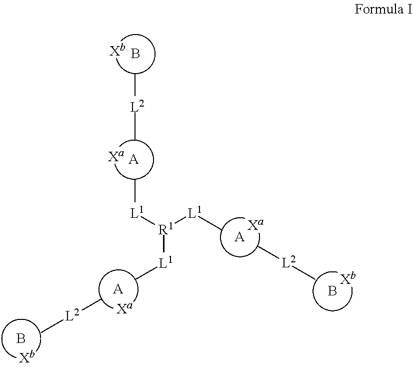

wherein R¹, L¹, A, X^{a}, L², B, X^{b} are as defined in any of statements 1 to 55; and M is selected from the group consisting of Zn²⁺, Mn²⁺, Cu²⁺, Fe²⁺, CO²⁺ and Ni²⁺.
80. A compound of Formula I shown below, or a pharmaceutically acceptable salt, hydrate or solvate thereof, in combination with a source of M, for use in the treatment of a proliferative disorder (e.g. cancer):

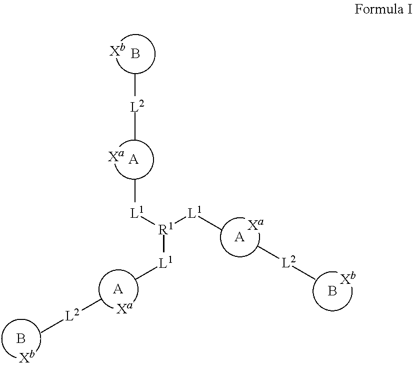

wherein R¹, L¹, A, X^{a}, L², B, X^{b} are as defined in any of statements 1 to 55; and M is selected from the group consisting of Zn²⁺, Mn²⁺, Cu²⁺, Fe²⁺, CO²⁺ and Ni²⁺.
81. The compound for use according to statement 79 or 80, wherein the compound has a structure according to any one of the following:

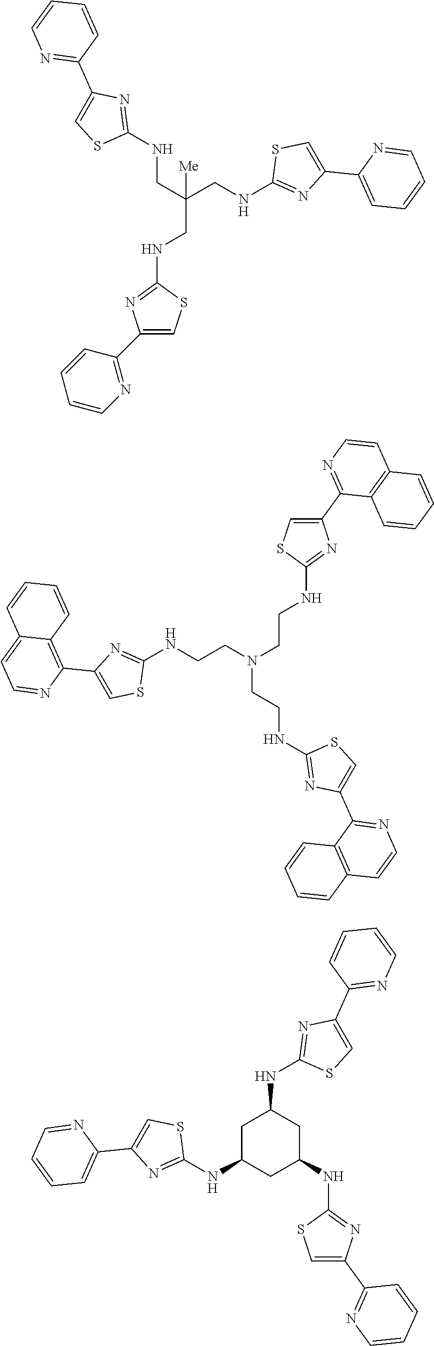

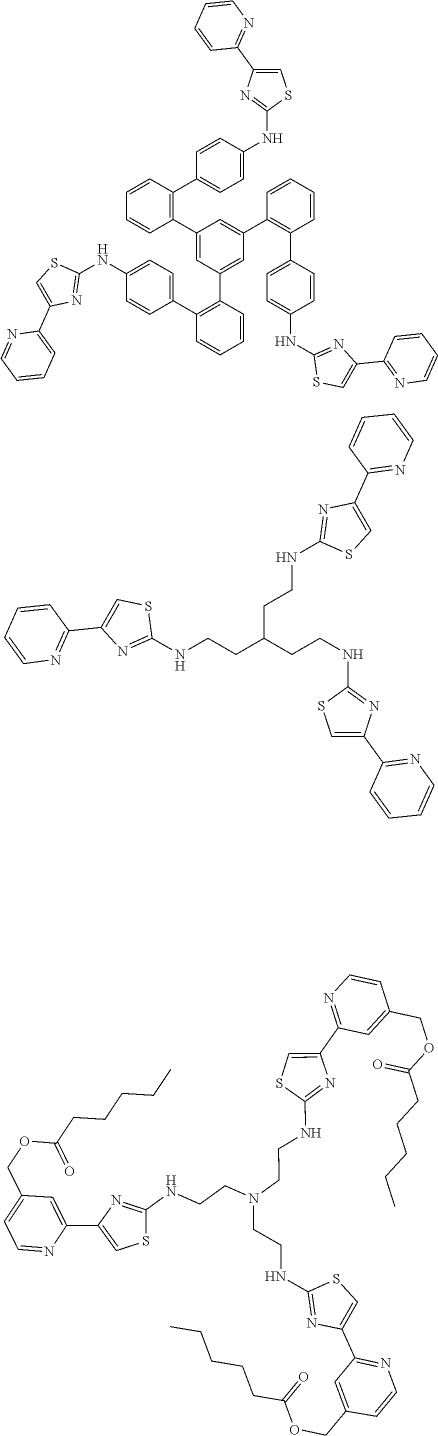

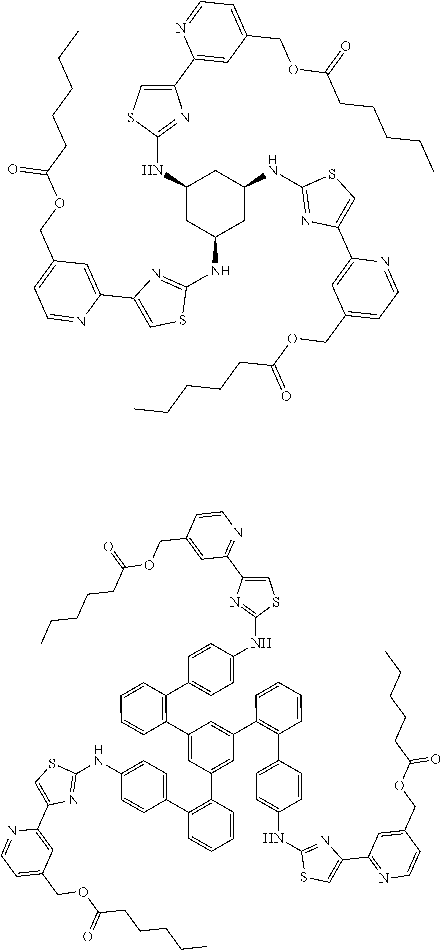 [SMILES: CCCCCC(=O)OCc1ccnc(-c2csc(N[C@H]3C[C@@H](Nc4nc(-c5cc(COC(=O)CCCCC)ccn5)cs4)C[C@@H](Nc4nc(-c5cc(COC(=O)CCCCC)ccn5)cs4)C3)n2)c1 ; CCCCCC(=O)OCc1ccnc(-c2csc(Nc3ccc(-c4ccccc4-c4cc(-c5ccccc5-c5ccc(Nc6nc(-c7cc(COC(=O)CCCCC)ccn7)cs6)cc5)cc(-c5ccccc5-c5ccc(Nc6nc(-c7cc(COC(=O)CCCCC)ccn7)cs6)cc5)c4)cc3)n2)c1]

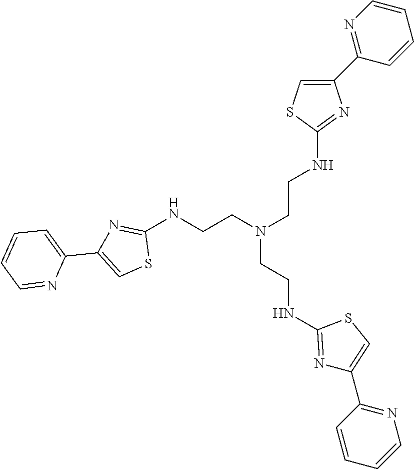 [SMILES: c1ccc(-c2csc(NCCN(CCNc3nc(-c4ccccn4)cs3)CCNc3nc(-c4ccccn4)cs3)n2)nc1]

82. The compound for use according to statement 79 or 80, wherein the compound has a structure according to the following:

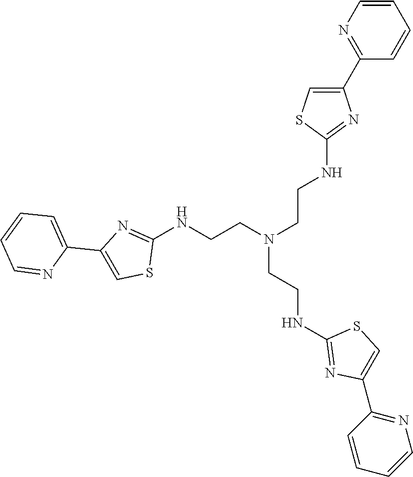 [SMILES: c1ccc(-c2csc(NCCN(CCNc3nc(-c4ccccn4)cs3)CCNc3nc(-c4ccccn4)cs3)n2)nc1]

83. The compound for use according to any one of statements 79 to 82, wherein the source of M is organic (e.g. M acetate or M propionate or M trifluoromethanesulfonate) or inorganic (e.g. M perchlorate or M nitrate or M tetrafluoroborate).
84. The compound for use according to any one of statements 79 to 83, wherein M is selected from the group consisting of Zn²⁺, Mn²⁺, Fe²⁺, CO²⁺ and Ni²⁺.
85. The compound for use according to any one of statements 79 to 83, wherein M is selected from the group consisting of Zn²⁺, Mn²⁺ and Fe²⁺.
86. The compound for use according to any one of statements 79 to 83, wherein M is selected from the group consisting of Zn²⁺ and Mn²⁺.
87. The compound for use according to any one of statements 79 to 86, wherein the compound of Formula I, or a pharmaceutically acceptable salt, hydrate or solvate thereof, is in further combination with a source of Q, wherein Q is an anion selected from the group consisting of spherical monoanionic anions, trigonal planar anions, dianionic tetrahedral anions, trianionic tetrahedral anions, dianionic octahedral anions and trianionic octahedral anions.
88. The compound for use according to statement 87, wherein Q is an anion selected from the group consisting of dianionic tetrahedral anions and trianionic tetrahedral anions.
89. The compound for use according to statement 87, wherein Q is an anion selected from the group consisting of dianionic tetrahedral oxoanions and trianionic tetrahedral oxoanions.
90. The compound for use according to statement 87, wherein Q is sulfate (SO₄²⁻), phosphate (PO₄³⁻) or organophosphate (such as monophenylphosphate).
91. The compound for use according to any one of statements 80 to 90, wherein the proliferative disorder is cancer.
92. The compound for use according to statement 91, wherein the cancer is selected from lung, colon, rectal, breast, ovarian, bladder, kidney, prostate, liver, pancreas, brain, bone, blood and skin cancer.
93. A compound of Formula II shown below, or a pharmaceutically acceptable salt, hydrate or solvate thereof, for use as a medicament:

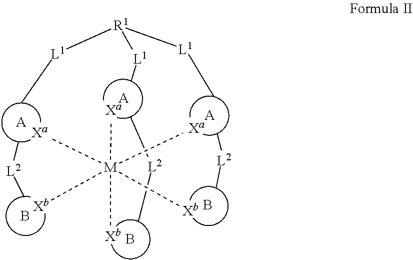

wherein R¹, L¹, A, X^{a}, L², B, X^{b} are as defined in any of statements 1 to 55; and M is selected from the group consisting of Zn²⁺, Mn²⁺, Cu²⁺, Fe²⁺, CO²⁺ and Ni²⁺.
94. A compound of Formula II shown below, or a pharmaceutically acceptable salt, hydrate or solvate thereof, for use as a medicament in the treatment of a proliferative disorder (e.g. cancer):

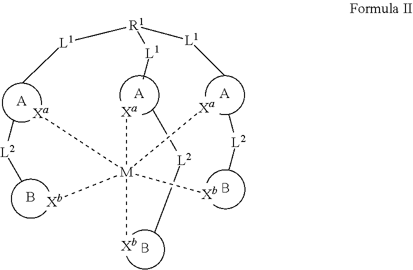

wherein R¹, L¹, A, X^{a}, L², B, X^{b} are as defined in any of statements 1 to 55; and M is selected from the group consisting of Zn²⁺, Mn²⁺, Cu²⁺, Fe²⁺, CO²⁺ and Ni²⁺.
95. The compound for use according to statement 93 or 94, wherein M is selected from the group consisting of Zn²⁺, Mn²⁺, Fe²⁺, CO²⁺ and Ni²⁺.
96. The compound for use according to statement 93 or 94, wherein M is selected from the group consisting of Zn²⁺, Mn²⁺ and Fe²⁺.
97. The compound for use according to statement 93 or 94, wherein M is selected from the group consisting of Zn²⁺ and Mn²⁺.
98. The compound for use according to any one of statements 93 to 97, wherein the compound of Formula II, or a pharmaceutically acceptable salt, hydrate or solvate thereof, is in combination with a source of Q, wherein Q is an anion selected from the group consisting of spherical monoanionic anions, trigonal planar anions, dianionic tetrahedral anions, trianionic tetrahedral anions, dianionic octahedral anions and trianionic octahedral anions.
99. The compound for use according to statement 98, wherein Q is an anion selected from the group consisting of dianionic tetrahedral anions and trianionic tetrahedral anions.
100. The compound for use according to statement 98, wherein Q is an anion selected from the group consisting of dianionic tetrahedral oxoanions and trianionic tetrahedral oxoanions.
101. The compound for use according to statement 98, wherein Q is sulfate (SO₄²⁻), phosphate (PO₄³⁻) or organophosphate (such as monophenylphosphate).
102. The compound for use according to any one of statements 94 to 101, wherein the proliferative disorder is cancer.
103. The compound for use according to statement 102, wherein the cancer is selected from lung, colon, rectal, breast, ovarian, bladder, kidney, prostate, liver, pancreas, brain, bone, blood and skin cancer.
104. A compound of Formula III shown below, or a pharmaceutically acceptable salt, hydrate or solvate thereof, for use as a medicament:

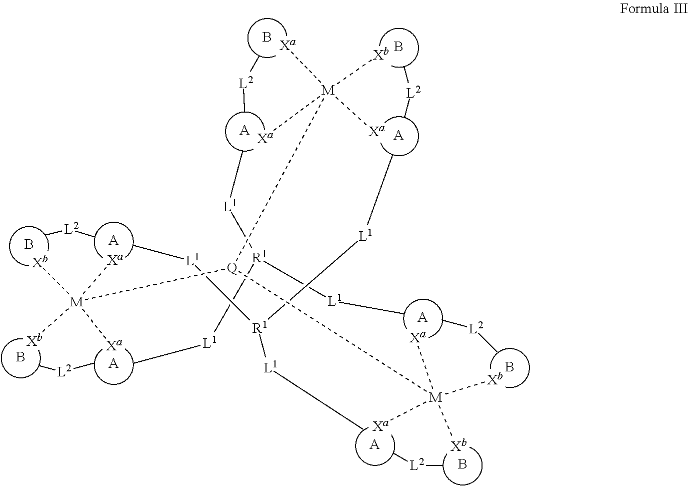

wherein
R¹, L¹, A, X^{a}, L², B, X^{b} are as defined in any one of statements 1 to 55;
M is selected from the group consisting of Zn²⁺, Mn²⁺, Cu²⁺, Fe²⁺, CO²⁺ and Ni²⁺;
Q is an anion selected from the group consisting of spherical monoanionic anions, trigonal planar anions, dianionic tetrahedral anions, trianionic tetrahedral anions, dianionic octahedral anions and trianionic octahedral anions.
105. A compound of Formula III shown below, or a pharmaceutically acceptable salt, hydrate or solvate thereof, for use in the treatment of a proliferative disorder (e.g. cancer):

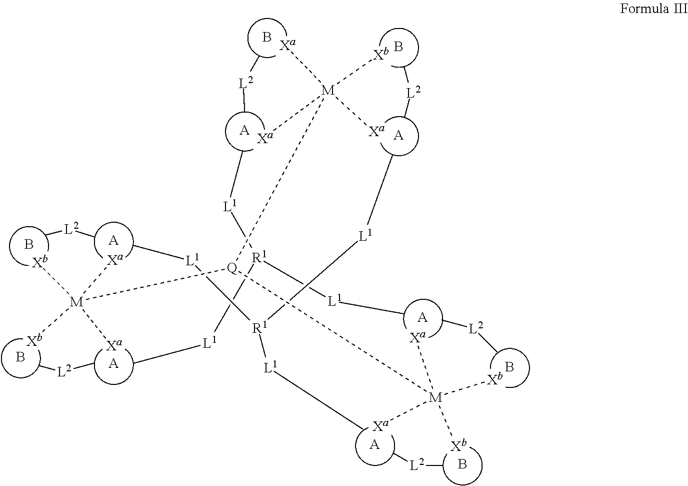

wherein
R¹, L¹, A, X^{a}, L², B, X^{b} are as defined in any one of statements 1 to 55;
M is selected from the group consisting of Zn²⁺, Mn²⁺, Cu²⁺, Fe²⁺, CO²⁺ and Ni²⁺;
Q is an anion selected from the group consisting of spherical monoanionic anions, trigonal planar anions, dianionic tetrahedral anions, trianionic tetrahedral anions, dianionic octahedral anions and trianionic octahedral anions.
106. The compound for use according to statement 104 or 105, wherein M is selected from the group consisting of Zn²⁺, Mn²⁺, Fe²⁺, CO²⁺ and Ni²⁺.
107. The compound for use according to statement 104 or 105, wherein M is selected from the group consisting of Zn²⁺, Mn²⁺ and Fe²⁺.
108. The compound for use according to statement 104 or 105, wherein M is selected from the group consisting of Zn²⁺ and Mn²⁺.
109. The compound for use according to any one of statements 104 to 108, wherein Q is an anion selected from the group consisting of dianionic tetrahedral anions and trianionic tetrahedral anions.
110. The compound for use according to any one of statements 104 to 108, wherein Q is an anion selected from the group consisting of dianionic tetrahedral oxoanions and trianionic tetrahedral oxoanions.
111. The compound for use according to any one of statements 104 to 108, wherein Q is sulfate (SO₄²⁻), phosphate (PO₄³⁻) or organophosphate (such as monophenylphosphate).
112. The compound for use according to any one of statements 105 to 111, wherein the proliferative disorder is cancer.
113. The compound for use according statement 112, wherein the cancer is selected from lung, colon, rectal, breast, ovarian, bladder, kidney, prostate, liver, pancreas, brain, bone, blood and skin cancer.
114. A kit of parts comprising:
   i) a compound of Formula I shown below, or a pharmaceutically acceptable salt, hydrate or solvate thereof; and
   ii) a source of M:

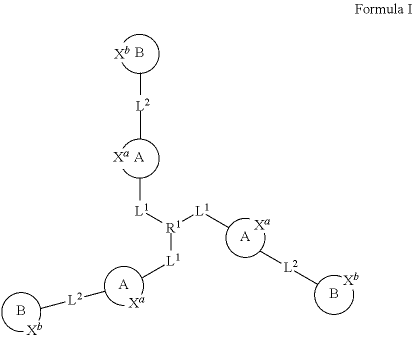

wherein
R¹, L¹, A, X^{a}, L², B, X^{b} are as defined in any one of statements 1 to 55; and
M is selected from the group consisting of Zn²⁺, Mn²⁺, Cu²⁺, Fe²⁺, CO²⁺ and Ni²⁺.
115. The kit of statement 114, further comprising means for facilitating compliance with a dosage regimen (e.g. instructions detailing how to administer the components of the kit).
116. The kit of statement 114 or 115, further comprising a source of Q, wherein Q is an anion selected from the group consisting of spherical monoanionic anions, trigonal planar anions, dianionic tetrahedral anions, trianionic tetrahedral anions, dianionic octahedral anions and trianionic octahedral anions.
117. A kit of parts comprising:
   i) a compound of Formula II shown below, or a pharmaceutically acceptable salt, hydrate or solvate thereof; and
   ii) a source of Q:

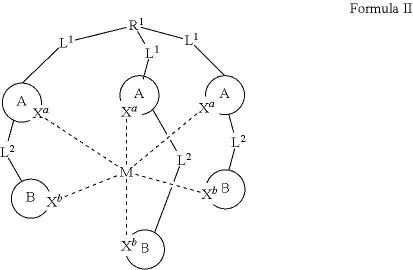

wherein
R¹, L¹, A, X^{a}, L², B, X^{b} are as defined in any one of statements 1 to 55;
M is selected from the group consisting of Zn²⁺, Mn²⁺, Cu²⁺, Fe²⁺, CO²⁺ and Ni²⁺; and
Q is an anion selected from the group consisting of spherical monoanionic anions, trigonal planar anions, dianionic tetrahedral anions, trianionic tetrahedral anions, dianionic octahedral anions and trianionic octahedral anions.
118. The kit of statement 117, further comprising means for facilitating compliance with a dosage regimen (e.g. instructions detailing how to administer the components of the kit).

### EXAMPLES

One or more examples of the invention will now be described, for the purpose of illustration only, with reference to the accompanying figures:

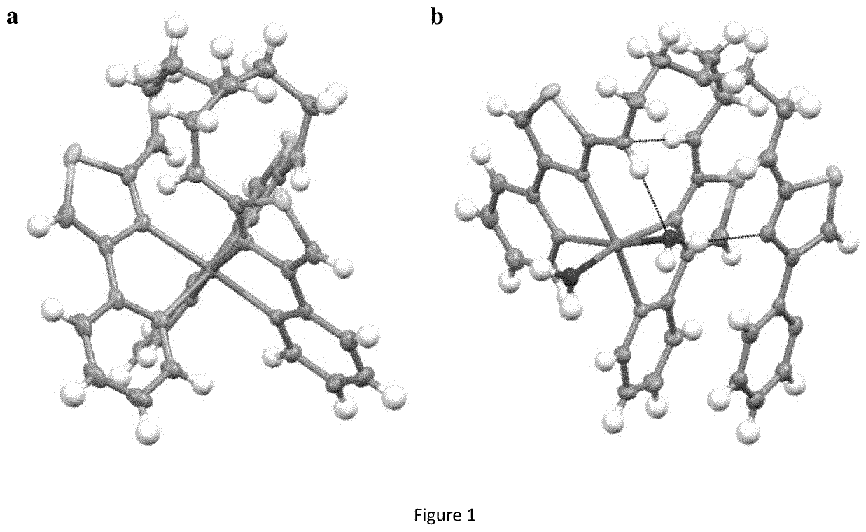

FIG. 1. Mononuclear complexes of L. a, structure of [LZn]²⁺. b, [LMn(H₂O)₂]²⁺.

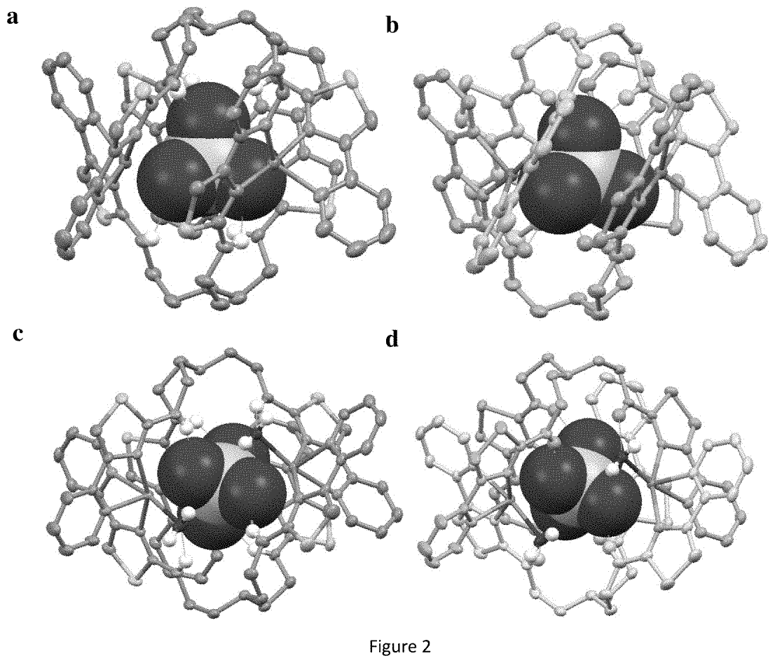

FIG. 2. Self-assembled metal-containing complexes of L incorporating SO₄²⁻. a, X-ray structure of [L₂Zn₃(SO₄)]⁴⁺. b, [L₂Zn₃(SO₄)]⁴⁺ with ligands coloured for clarity. c, X-ray structure of [L₂Mn₃(H₂O)₂(SO₄)]⁴⁺. d, [L₂Mn₃(H₂O)₂(SO₄)]⁴⁺ with ligands coloured for clarity.

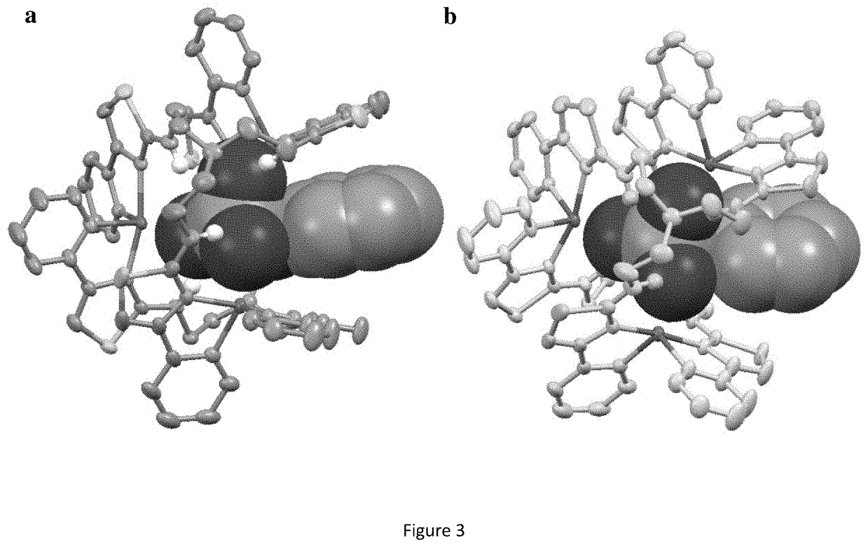

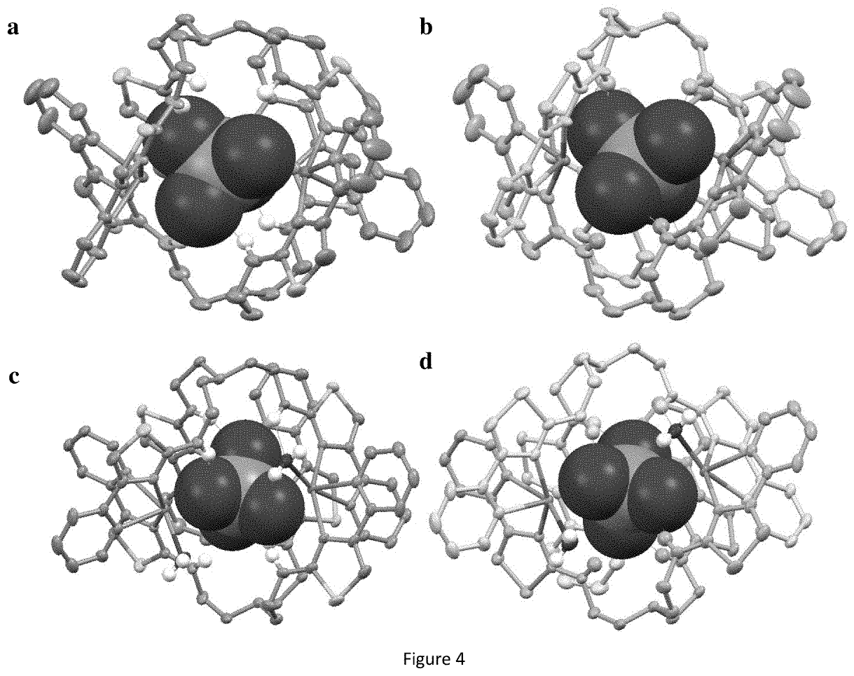

FIG. 3. Self-assembled Cu²⁺ complexes of L incorporating PhOPO₃²⁻. a, X-ray structure of [L₂Cu₃(PhOPO₃)]⁴⁺. b, [L₂Cu₃(PhOPO₃)]⁴⁺ with ligands coloured for clarity.

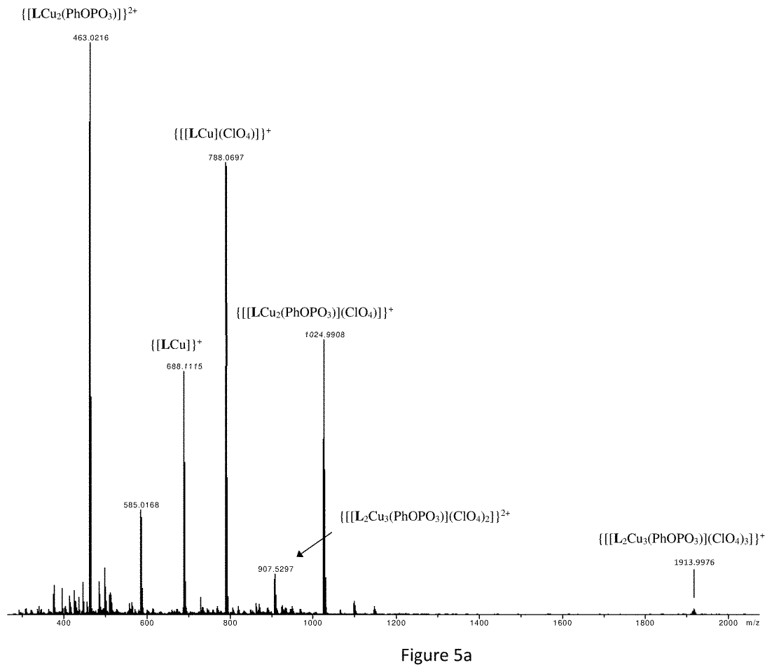

FIG. 4. Reaction of [L₂Zn₃]⁶⁺ and [L₂Mn₃]⁶⁺ with PhOPO₃²⁻. a, X-ray structure of [L₂Zn₃ (PO₄)]³⁺. b, [L₂Zn₃(PO₄)]³⁺ with ligands coloured for clarity. c, X-ray structure of [L₂Mn₃ (H₂O)₂(PO₄)]³⁺. d, [L₂Mn₃(H₂O)₂(PO₄)]³⁺ with ligands coloured for clarity.

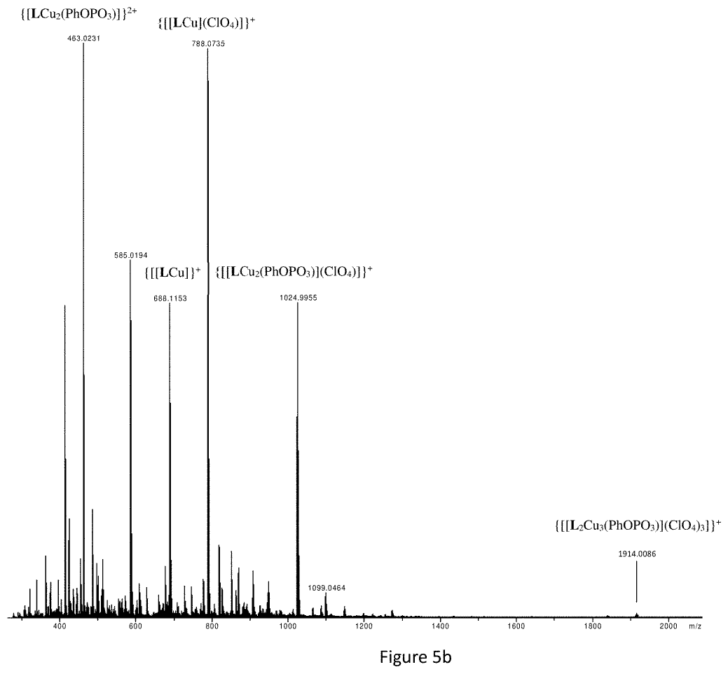

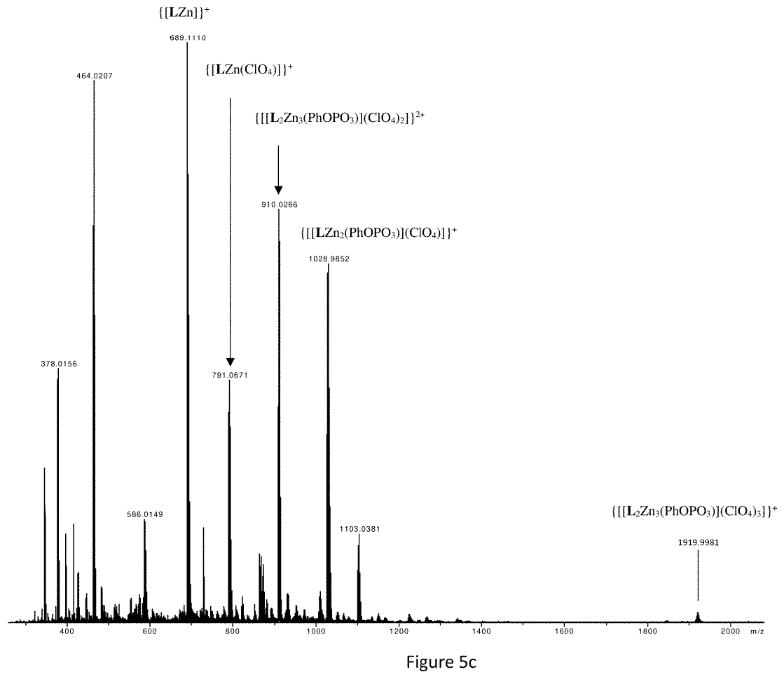

FIG. 5. a, ESI-MS of [L₂Cu₃(PhOPO₃)](ClO₄)₄. b, ESI-MS of [L₂Cu₃(PhOPO₃)](ClO₄)₄ after being heated at 80° C. for 1 hour. c, ESI-MS of [L₂Zn₃(PhOPO₃)](ClO₄)₄. d, ESI-MS of [L₂Zn₃(PhOPO₃)](ClO₄)₄ after being heated at 80° C. for 1 hour.

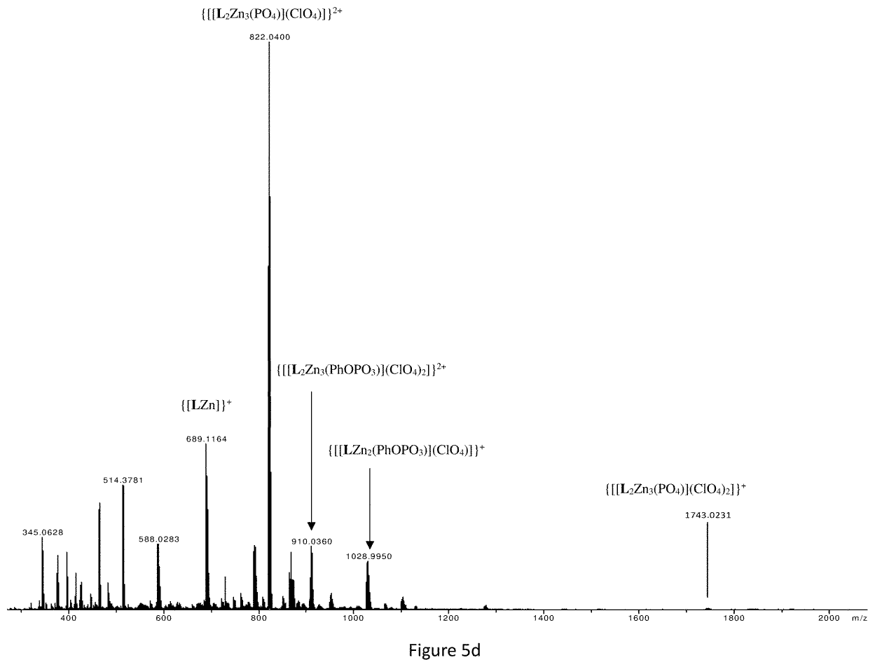

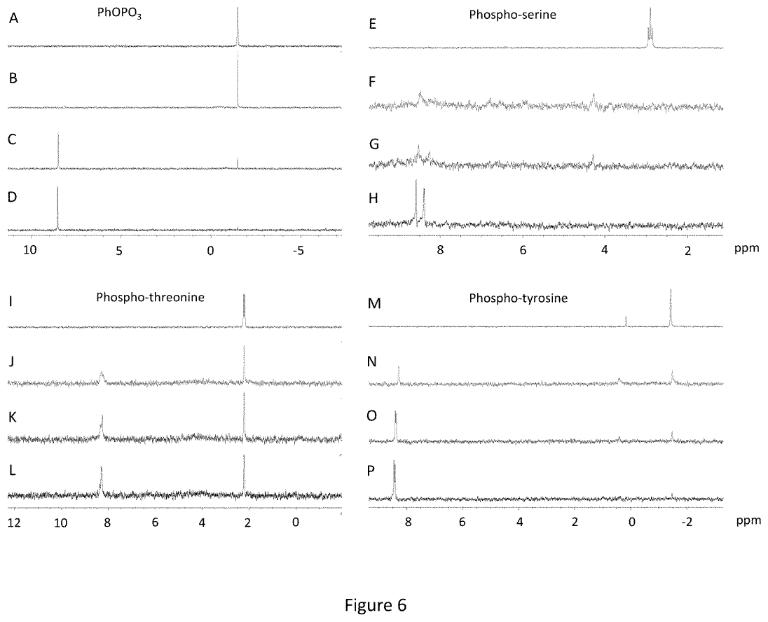

FIG. 6. Phosphatase activity of [L₂Zn₃]⁶⁺. ³¹P NMR spectra using different substrates including PhOPO₃ (spectra A to D), serine phosphate (spectra E to H), threonine phosphate (spectra I to L) and tyrosine phosphate (spectra M to P). Specific details for each ³¹P NMR spectra are as follows: Spectra A, E, I and M represent substrate alone (44 hrs incubated @ 37° C.); Spectra B, F, J and N represents [L₂Zn₃]⁶⁺ plus substrate (t=0 min); Spectra C, G, K and O represents [L₂Zn₃]⁶⁺ plus substrate incubated @ 37° C. for 19 hours; Spectra D, H, L and M represents [L₂Zn₃]⁶⁺ plus substrate incubated @ 37° C. for 44 hours. The ³¹P NMR of [L₂Zn₃(PO₄)]³⁺ gives a signal at 8.6 ppm. The doubling up of some of the ³¹P signals in [L₂Zn₃(PO₄)]³⁺ (H, P and L) is attributed to formation of a mixture of diastereoisomers between the racemic cryptand and the resolved chiral amino acids which will form an ion-pair ([L₂Zn₃(PO₄)](RCH(NH₂)CO₂)²⁺) and does not occur with the achiral phenyl phosphate.

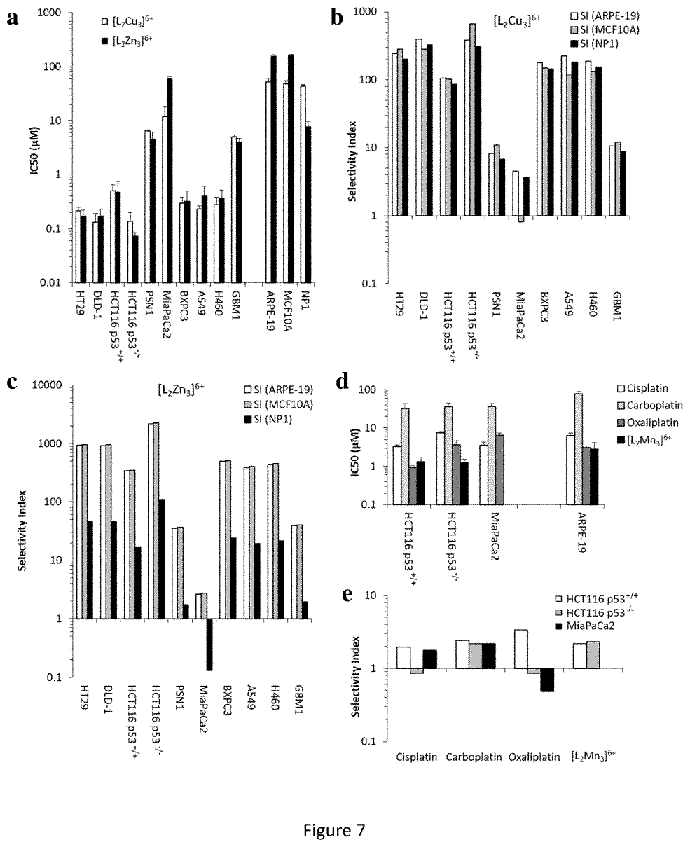

FIG. 7. Chemosensitivity response of a panel of human cancer and non-cancer cell lines to 96 h continuous exposure to self-assembling test compounds. a, The potency of compounds tested against cancer (HT-29, DLD-1, HCT116 p53^{+/+} and p53+, PSN1, MiaPaCa2, BxPC3, A549, H460 and GBM1) and non-cancer cells (ARPE-19, MCF10A and NP1). Each value represents the mean IC₅₀±standard deviation from a minimum of three independent experiments. b,c, The selectivity index (SI) for [L₂Cu₃]⁶⁺ (b) and [L₂Zn₃]⁶⁺ (c) for the indicated cancer cell lines; SI is defined as the mean IC₅₀ against the particular non-cancer cell line model divided by the mean IC₅₀ against the particular cancer cell line. SI values >1 indicate that the test compound is more active against the particular cancer cell line than the corresponding non-cancer cells. As the SI value is calculated using the mean IC₅₀ values, experimental error is not included in these figures. d,e, IC₅₀ values for the clinically approved platinates (cisplatin, oxaliplatin and carboplatin) and [L₂Mn₃]⁶⁺ and the corresponding SI results.

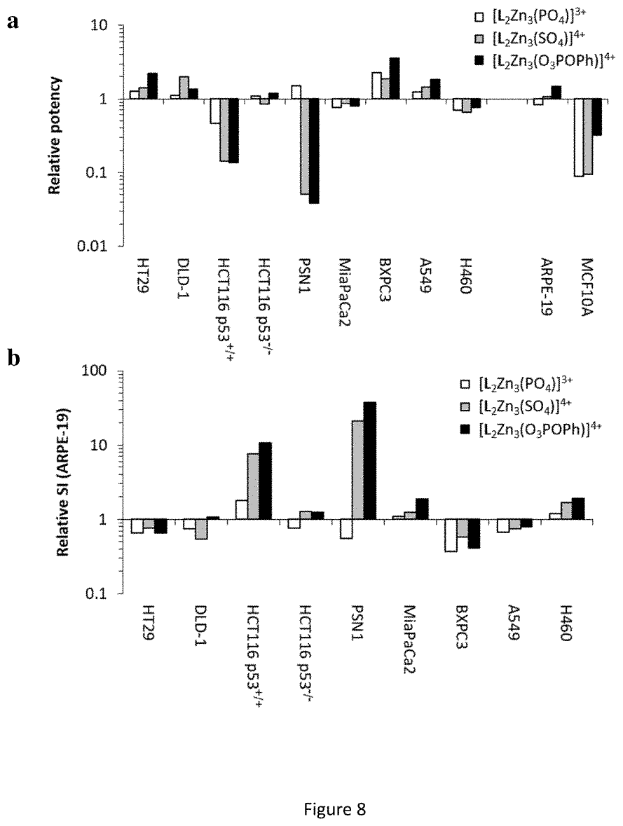

FIG. 8. Effect of the complex anion on potency and selectivity. a, The effect of various anions on the potency of the Zn²⁺ complex; this is expressed as relative potency which is defined as the IC₅₀ of [L₂Zn₃(PO₄)]³⁺, [L₂Zn₃ (SO₄)]⁴⁺ or [L₂Zn₃ (O₃POPh)]⁴⁺ divided by the IC₅₀ of [L₂Zn₃]⁶⁺ (values <1 and >1 indicate increased and decreased potency respectively). b, Effects of the anion on selectivity; these results are expressed as relative SI defined as the SI of [L₂Zn₃(PO₄)]³⁺, [L₂Zn₃(SO₄)]⁴⁺ or [L₂Zn₃(O₃POPh)]⁴⁺ divided by the SI of [L₂Zn₃]⁶⁺ (values >1 and <1 indicate increased and decreased selectivity respectively).

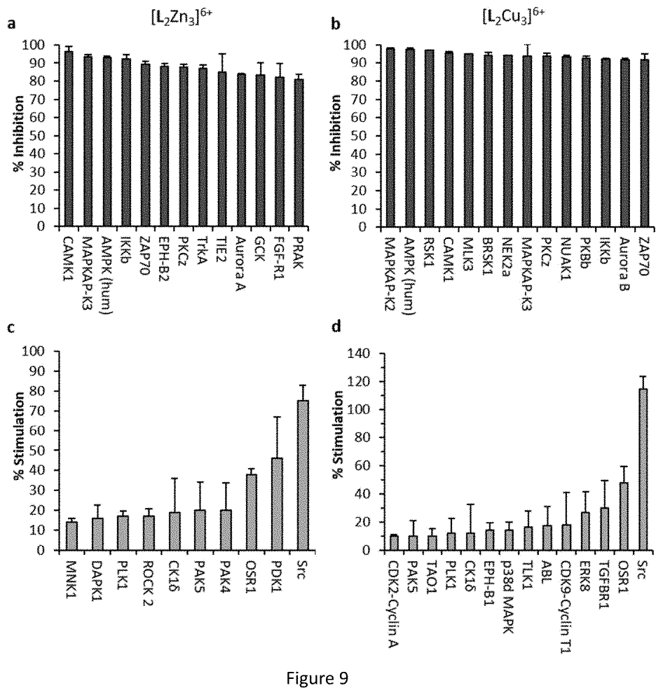

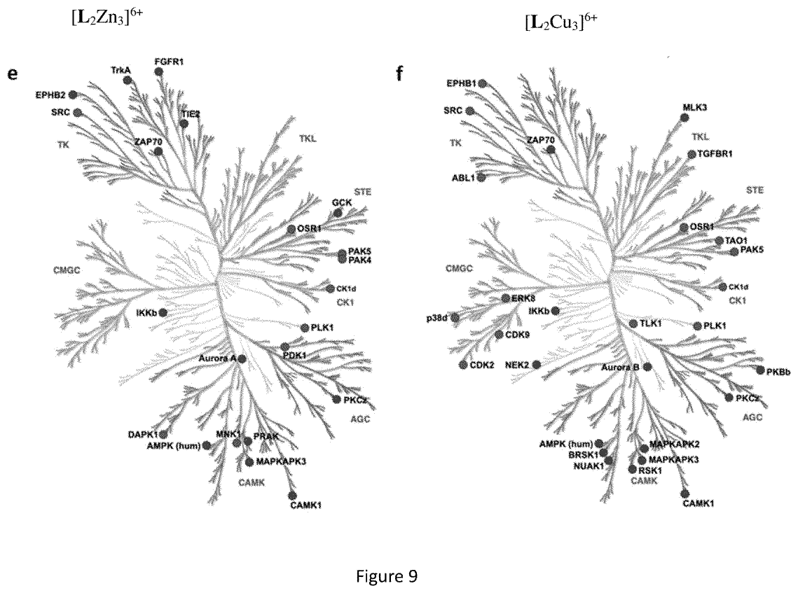

FIG. 9. Effects of [L₂Zn₃]⁶⁺ and [L₂Cu₃]⁶⁺ on the activity of purified human kinases. a,b, Percentage inhibition of the indicated kinases by [L₂Zn₃]⁶⁺ (a) and [L₂Cu₃]⁶⁺ (b) respectively at a concentration of 10 μM; full results are presented in the ESI. c,d, Kinases whose activity is stimulated by [L₂Zn₃]⁶⁺ (c) or [L₂Cu₃]⁶⁺ (d) complexes. e,f, Kinome map showing kinases inhibited (red) or stimulated (green) by [L₂Zn₃]⁶⁺ (e) or [L₂Cu₃]⁶⁺ (f). III ustration reproduced courtesy of Cell Signalling Technology Inc (www.cellsignal.com)

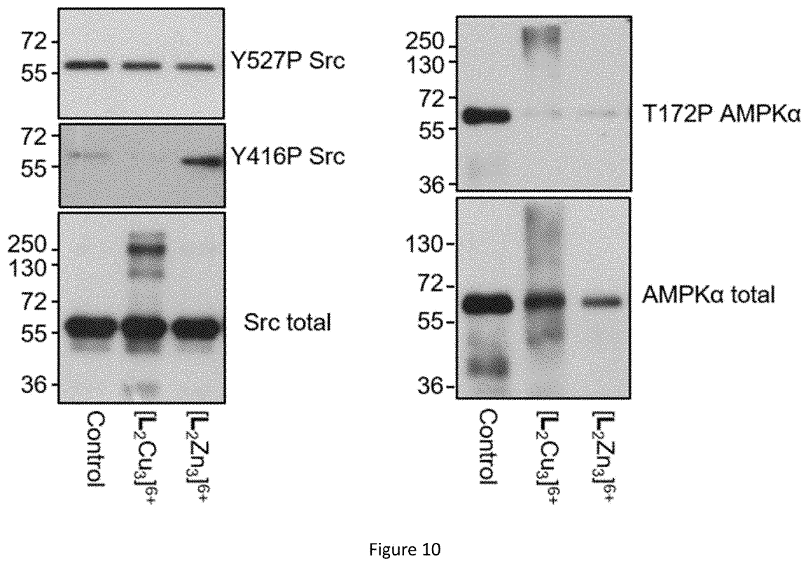

FIG. 10. Western blot analysis of purified recombinant Src and AMPK following exposure to [L₂Zn₃]⁶⁺ and [L₂Cu₃]⁶⁺. Purified enzymes were incubated with complexes (50 μM) for 4 hours in the presence of ATP prior to analysis.

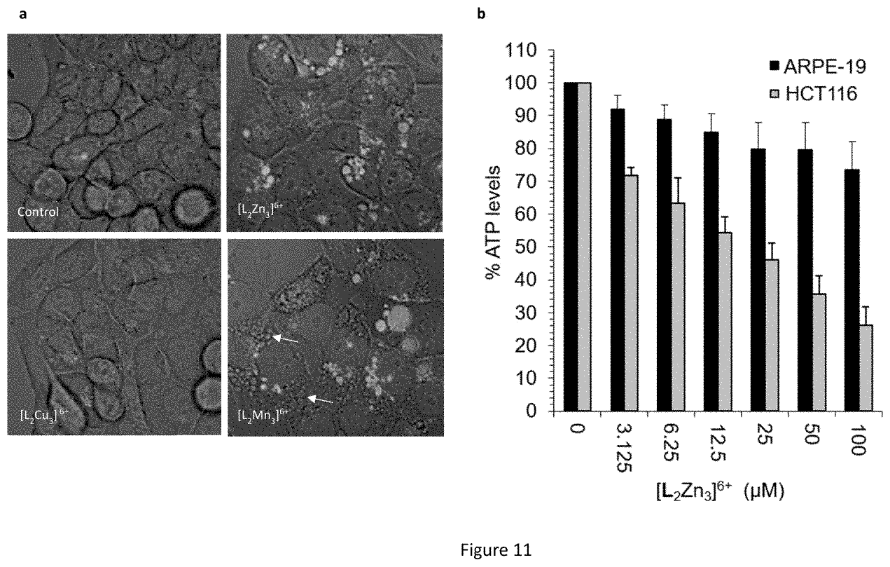

FIG. 11. Effects of [L₂Zn₃]⁶⁺, [L₂Cu₃]⁶⁺ and [L₂Mn₃]⁶⁺ complexes on autophagy and cellular ATP levels. a, Representative fluorescent images showing CYTO-ID autophagic staining (green) and Hoechst (blue) with bright field overlay of HCT116 p53^{-/-} cells treated with 3.125 μM [L₂Zn₃]⁶⁺, [L₂Cu₃]⁶⁺ or [L₂Mn₃]⁶⁺ for 40 h. White arrows indicate intracellular vacuoles. b, Cellular ATP levels in ARPE19 non-cancer cells and HCT116 p53^{+/+} cancer cells with 20 h exposure to [L₂Zn₃]⁶⁺ at the indicated concentrations.

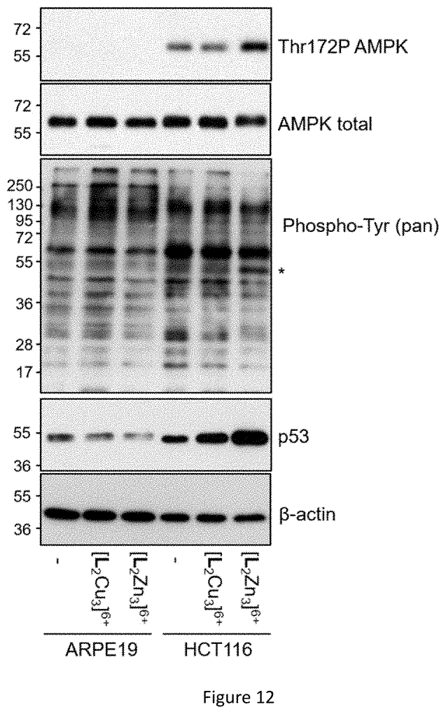

FIG. 12. Immunoblots showing the differential effects of [L₂Cu₃]⁶⁺ and [L₂Zn₃]⁶⁺ treatment of HCT116 cancer cells and ARPE19 non-cancer cells on key cellular proteins associated with cellular and metabolic stress. Representative immunoblot images of the indicated proteins following cell exposure to 5 μM [L₂Cu₃]⁶⁺ or [L₂Zn₃]⁶⁺ for 40 h. Tyrosine phosphorylated proteins are indicated using a pan-phospho-Tyr antibody; β-actin as a loading control.

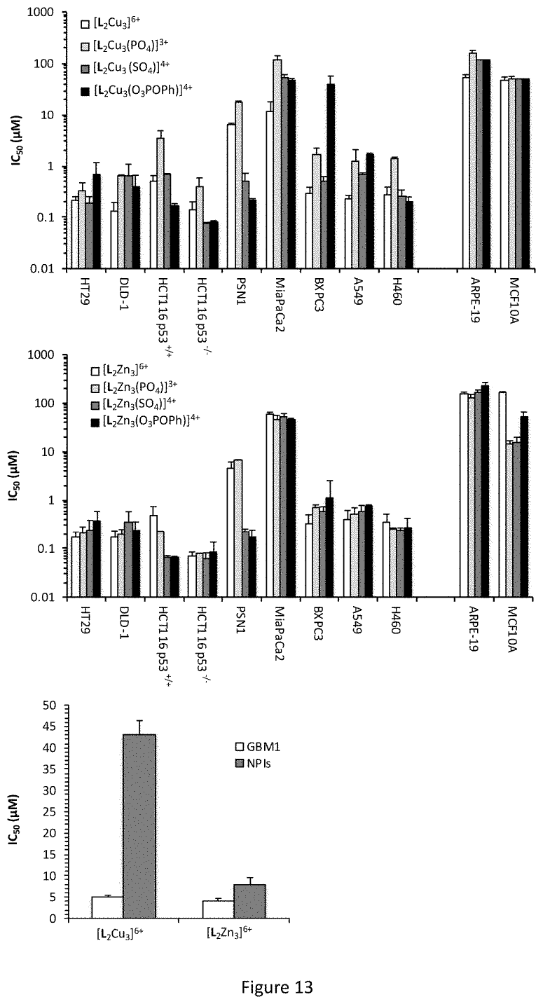

FIG. 13. The effect of anions of the potency of [L₂Cu₃]⁶⁺ and [L₂Zn₃]⁶⁺. The results represent the mean IC₅₀ values ±standard deviation for at least three independent experiments.

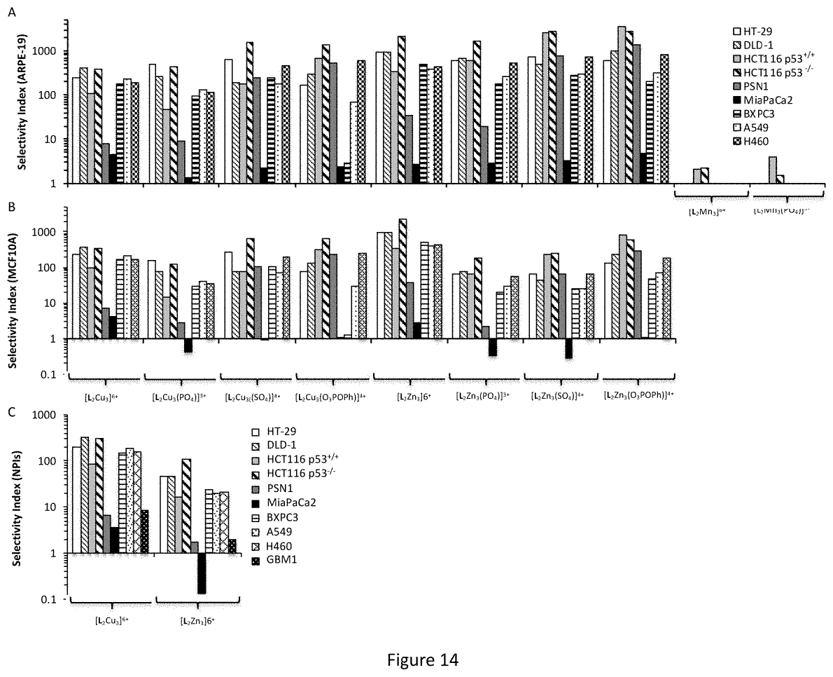

FIG. 14. The effect of anions on the selectivity of [L₂Cu₃]⁶⁺ and [L₂Zn₃]⁶⁺. All values presented here were determined from the mean IC₅₀ values in FIG. 13 for each of the non-cancer cell lines used in this study. As a result, no error bars are presented here as the experimental error is accounted for in FIG. 13.

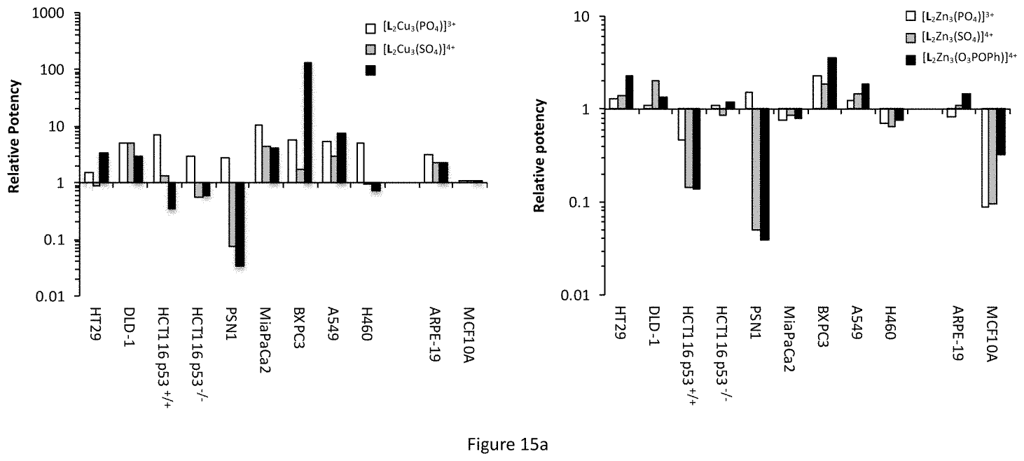

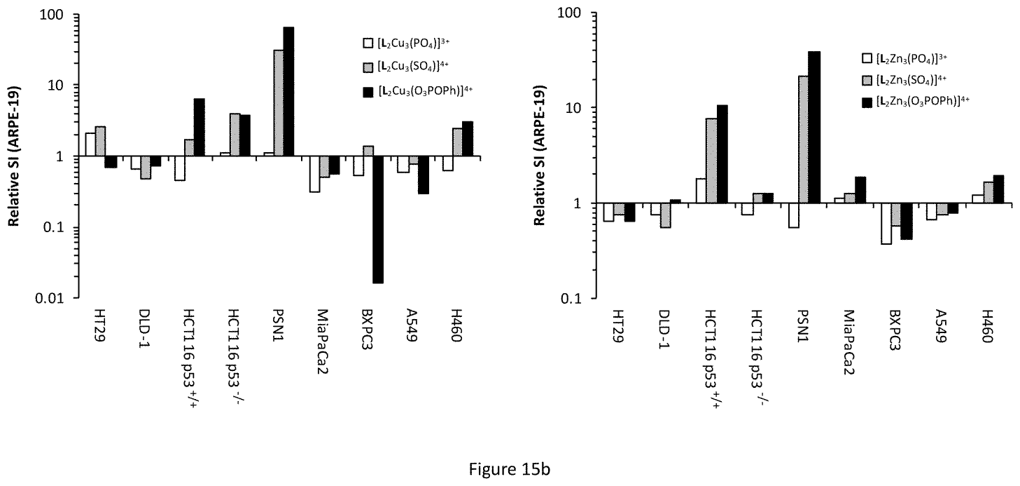

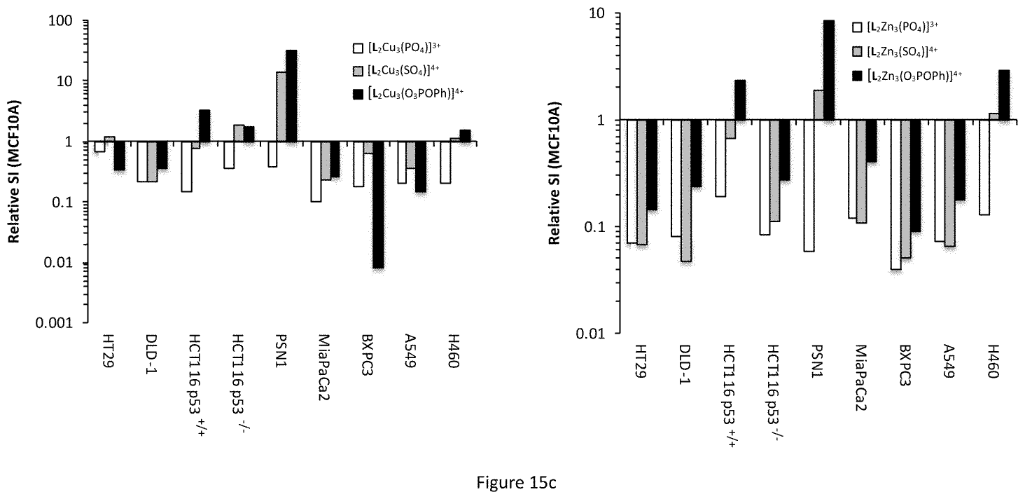

FIG. 15. The effect of anions on the potency and selectivity relative to [L₂Cu₃]⁶⁺ and [L₂Zn₃]⁶⁺. Relative potency was determined by dividing the IC₅₀ of test compounds plus respective anions divided by IC₅₀ values for [L₂Cu₃]⁶⁺ and [L₂Zn₃]⁶⁺. Values >1 represent an increase in potency and conversely, values <1 represent a reduction in potency. Relative selectivity index (SI) values were determined by dividing the SI value for test compounds plus respective anions divided by SI values for [L₂Cu₃]⁶⁺ and [L₂Zn₃]⁶⁺. Values >1 represent an increase in selectivity and conversely, values <1 represent a reduction in selectivity.

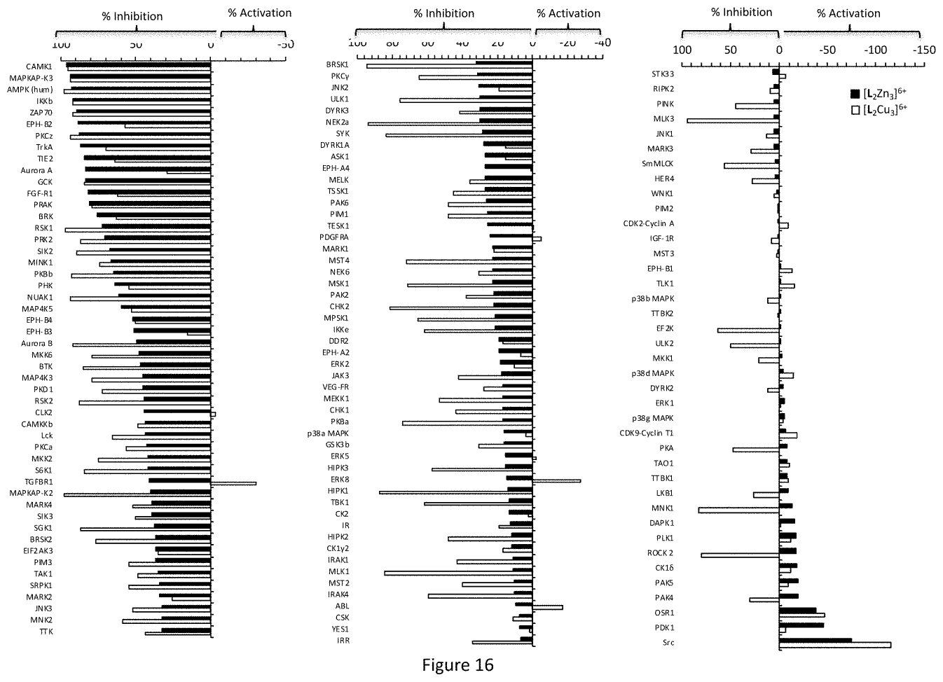

FIG. 16. The effect of [L₂Zn₃]⁶⁺ and [L₂Cu₃]⁶⁺ on the activity of recombinant human kinases. The compounds were submitted to the MRC Protein Phosphorylation and Ubiquitination Unit International Centre for Kinase Profiling (University of Dundee) and tested at a concentration of 10 μM against 140 human kinases (Premier Screen).

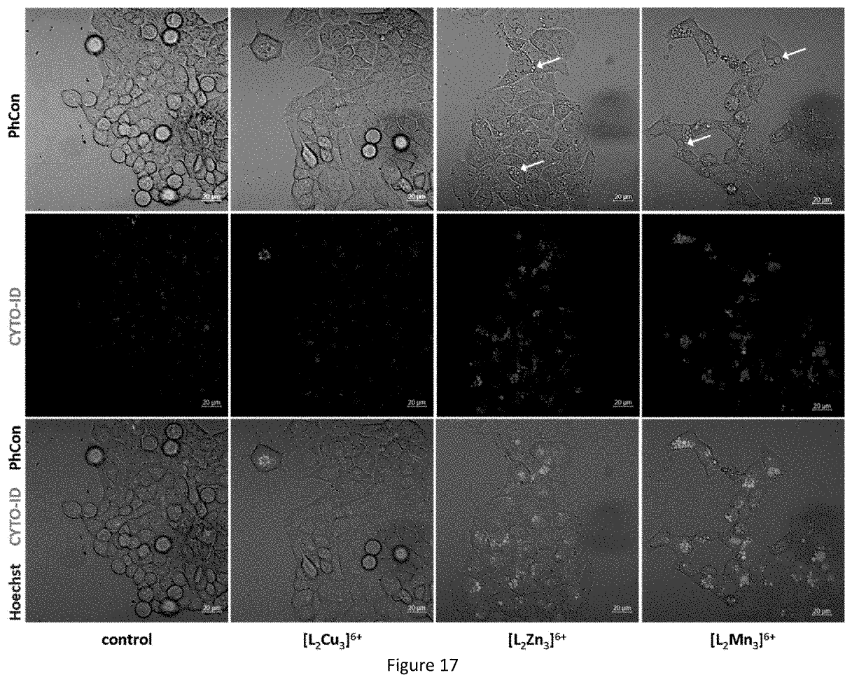

FIG. 17. The effects of [L₂Cu₃]⁶⁺, [L₂Zn₃]⁶⁺ and [L₂Mn₃]⁶⁺ on cellular vacuole formation and autophagy in the HCT116 p53^{-/-} cancer cells. Representative confocal images are shown of HCT116 p53^{-/-} cancer cells following 40 h treatment with vehicle control or 3.125 μM [L₂Cu₃]⁶⁺, [L₂Zn₃]⁶⁺, or [L₂Mn₃]⁶⁺. Upper panel shows phase contrast cell images with white arrows indicating intracellular vacuoles. Middle panel shows cells staining positive for autophagy (CYTO-ID autophagic dye, green). Lower panel shows overlay of phase contrast cell images with CYTO-ID autophagic staining (green, punctate, cytoplasmic) and counterstaining of nuclei (Hoechst).

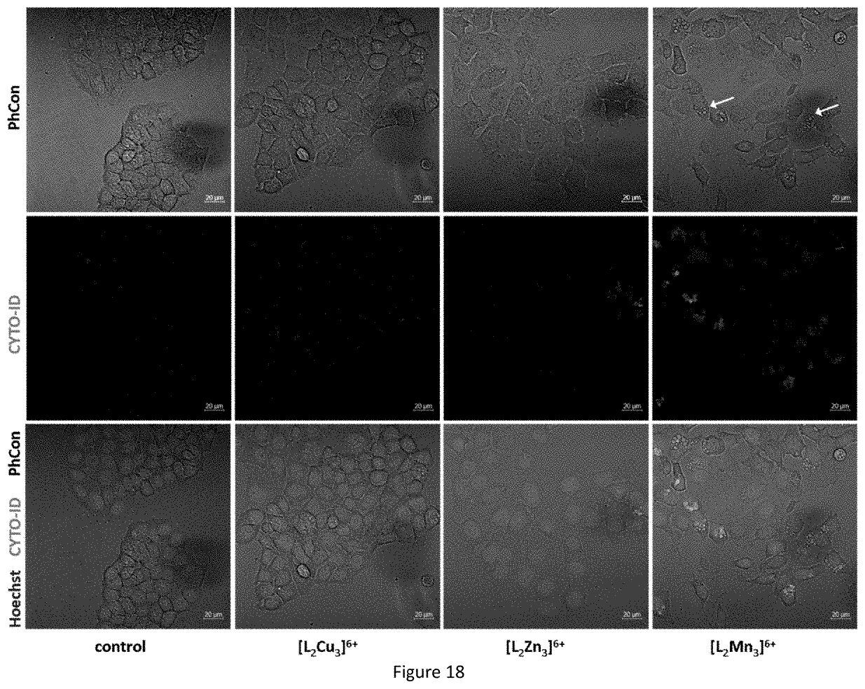

FIG. 18. The effects of [L₂Cu₃]⁶⁺, [L₂Zn₃]⁶⁺ and [L₂Mn₃]⁶⁺ on cellular vacuole formation and autophagy in the HCT116 p53^{+/+} cancer cells. Representative confocal images are shown of HCT116 p53^{+/+} cancer cells following 40 h treatment with vehicle control or 3.125 μM [L₂Cu₃]⁶⁺, [L₂Zn₃]⁶⁺, or [L₂Mn₃]⁶⁺. Upper panel shows phase contrast cell images with white arrows indicating intracellular vacuoles. Middle panel shows cells staining positive for autophagy (CYTO-ID autophagic dye, green). Lower panel shows overlay of phase contrast cell images with CYTO-ID autophagic staining (green, punctate, cytoplasmic) and counterstaining of nuclei (Hoechst).

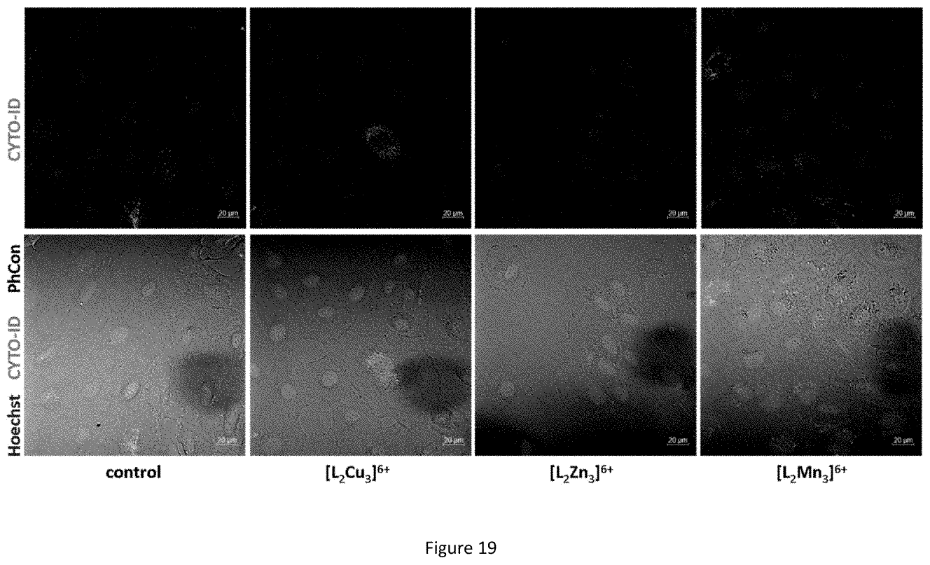

FIG. 19. The effects of [L₂Cu₃]⁶⁺, [L₂Zn₃]⁶⁺ and [L₂Mn₃]⁶⁺ on cellular vacuole formation and autophagy in the ARPE19 non-cancer cells. Representative confocal images are shown of ARPE19 non-cancer cells following 40 h treatment with vehicle control or 3.125 μM [L₂Cu₃]⁶⁺, [L₂Zn₃]⁶⁺, or [L₂Mn₃]⁶⁺. Upper panel shows cells staining positive for autophagy (CYTO-ID autophagic dye, green). Lower panel shows overlay of phase contrast cell images with CYTO-ID autophagic staining (green, punctate, cytoplasmic) and counterstaining of nuclei (Hoechst).

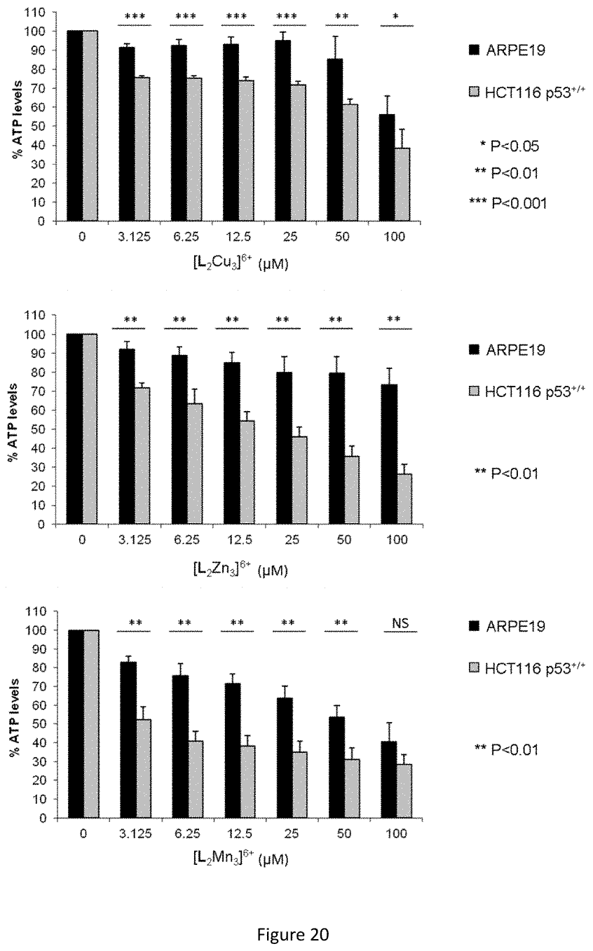

FIG. 20. The effects of [L₂Cu₃]⁶⁺, [L₂Zn₃]⁶⁺ and [L₂Mn₃]⁶⁺ on cellular ATP levels in the ARPE19 non-cancer and HCT116 p53^{+/+} cancer cells. The effects of 20 h treatment with the indicated concentrations of [L₂Cu₃]⁶⁺, [L₂Zn₃]⁶⁺, or [L₂Mn₃]⁶⁺ on total cellular levels of ATP compared to levels in vehicle control-treated ARPE19 or HCT116^{+/+} cells. n=3 biological repeats, students t-test (p values).

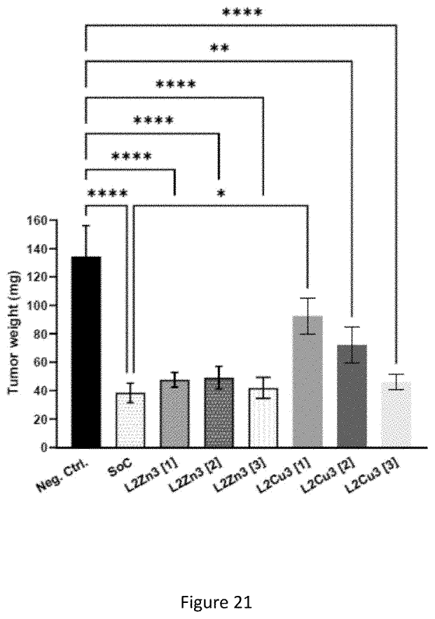

FIG. 21. Activity of [L₂Cu₃]⁶⁺ and [L₂Zn₃]⁶⁺ against primary H460 tumors. The results presented represent the mean tumour weight±SEM following treatment at 3 different doses of [L₂Cu₃]⁶⁺ and [L₂Zn₃]⁶⁺: L2Zn3 [1]=54 μM, L2Zn3 [2]=108 μM, L2Zn3 [3]=216 μM; L2Cu3 [1]=42 μM, L2Cu3[2]=84 μM, L2Cu3[3]=168 μM. SoC paclitaxel. Statistical analysis by one way ANOVA (*): 0.05≥p value >0.01; (**): 0.01≥p value >0.001; (***): 0.001≥p value; (****): 0.0001≥p value. Number per experimental group n=10-13.

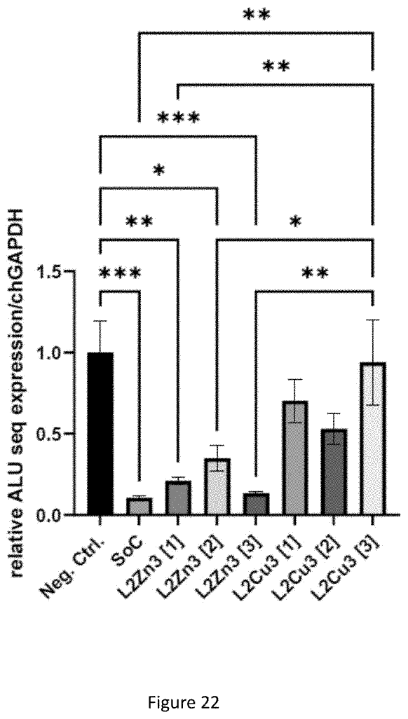

FIG. 22. Evaluation of the effects of [L₂Cu₃]⁶⁺ and [L₂Zn₃]⁶⁺ on primary H460 metastasis to the lower CAM. Relative ALU expression (relative to chicken GAPDH) as a quantitive marker of metastasis of human cells to the lower CAM. L2Zn3 [1]=54 μM, L2Zn3 [2]=108 μM, L2Zn3 [3]=216 μM; L2Cu3 [1]=42 μM, L2Cu3[2]=84 μM, L2Cu3[3]=168 μM. SoC paclitaxel. Relative expression ±SEM. Statistical analysis by one way ANOVA (*): 0.05≥p value >0.01; (**): 0.01≥p value >0.001; (***): 0.001≥p value; (****): 0.0001≥p value. Number per experimental group n=7-8.

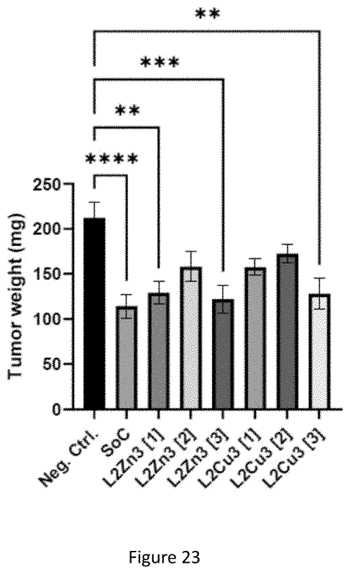

FIG. 23. Activity of [L₂Cu₃]⁶⁺ and [L₂Zn₃]⁶⁺ against primary HT29 tumors. The results presented represent the mean tumour weight ±SEM following treatment at 3 different doses of [L₂Cu₃]⁶⁺ and [L₂Zn₃]⁶⁺: L2Zn3 [1]=26.6 μM, L2Zn3 [2]=53.2 μM, L2Zn3 [3]=106.4 μM; L2Cu3 [1]=23.8 μM, L2Cu3[2]=65.7 μM, L2Cu3[3]=131.46 μM. SoC doxorubicin. Statistical analysis by one way ANOVA (*): 0.05≥p value >0.01; (**): 0.01≥p value >0.001; (***): 0.001≥p value; (****): 0.0001≥p value. Number per experimental group n=10-14.

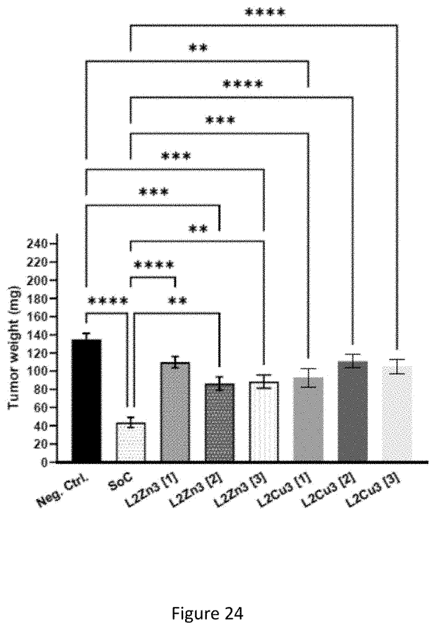

FIG. 24. Activity of [L₂Cu₃]⁶⁺ and [L₂Zn₃]⁶⁺ against primary HCT116 tumors. The results presented represent the mean tumour weight ±SEM following treatment at 3 different doses of [L₂Cu₃]⁶⁺ and [L₂Zn₃]⁶⁺: L2Zn3 [1]=70.5 μM, L2Zn3 [2]=141 μM, L2Zn3 [3]=282 μM; L2Cu3 [1]=75 μM, L2Cu3[2]=150 μM, L2Cu3[3]=300 μM. SoC doxorubicin. Statistical analysis by one way ANOVA (*): 0.05≥p value >0.01; (**): 0.01≥p value >0.001; (***): 0.001≥p value; (****): 0.0001≥p value. Number per experimental group n=12-15.

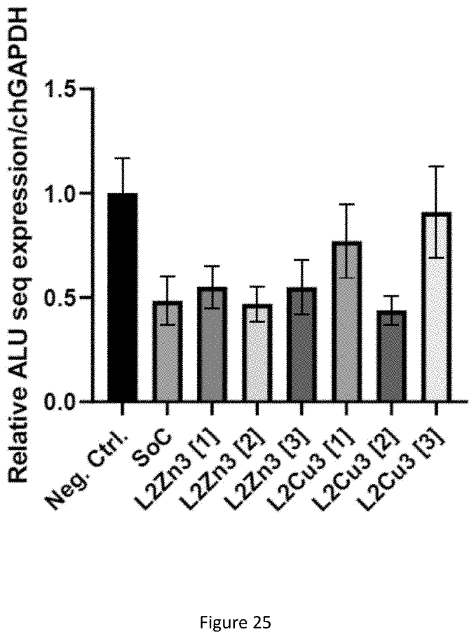

FIG. 25. Evaluation of the effects of [L₂Cu₃]⁶⁺ and [L₂Zn₃]⁶⁺ on primary HCT116 metastasis to the lower CAM. Relative ALU expression (relative to chicken GAPDH) as a quantitive marker of metastasis of human cells to the lower CAM. Relative expression ±SEM. L2Zn3 [1]=70.5 μM, L2Zn3 [2]=141 μM, L2Zn3 [3]=282 μM; L2Cu3 [1]=75 μM, L2Cu3[2]=150 μM, L2Cu3[3]=300 μM. SoC doxorubicin. Number per experimental group n=7-8.

### PART A

### Synthesis and Characterisation

Unless otherwise stated, all solvents and materials were purchased from either Sigma Aldrich, Fisher Scientific or Fluorochem and were used without further purification. ¹H, ¹³C, DEPT-135 and DEPT-90 NMR data was recorded on either a Bruker Fourier 300 MHz or Bruker Avance III (AVIII) 400 MHz spectrometer or a Bruker Avance Neo 600 MHz NMR spectrometer. Mass spectra were obtained on an Agilent 6210 TOF MS with electrospray ionisation operating in positive ion mode and mass spectra of metal complexes were obtained on a Bruker Micro TOF-q LC mass spectrometer with electrospray ionisation operating in positive ion mode. Phosphorylated ‘SAMS’ peptide HMRSAMS*GLHLVKRR (phosphorylated on the serine residue) was obtained from PeptideSynthetics, Peptide Protein Research Ltd (>95% purity).

Single crystal X-ray diffraction data was collected at 150(2) K on a Bruker D8 Venture diffractometer equipped with a graphite monochromated Mo(Kα) radiation source and a cold stream of N2 gas. Solutions were generated by conventional heavy atom Patterson or direct methods and refined by full-matrix least squares on all F² data, using SHELXS-97 and SHELXL software respectively.²⁶ Absorption corrections were applied based on multiple and symmetry-equivalent measurements using SADABS.²⁷ Almost all the structures contained some form of disordered ether with solvent molecules and/or counter anions (generally substitutional or rotation disorder). In these cases, the atoms were modelled using the PART instruction in the least squares refinement and refined over two positions. The anisotropic displacement parameters were treated with S/MU, DELU and in some cases ISOR where needed. Due to the diffuse nature of the electron density map the hydrogen atoms were not added to disordered solvent molecules. The structure [L₂Zn₃SO₄](BF₄)_{3.5} contained extensively disordered tetrafluoroborate counter anions, one of which refined poorly and was modelled with 50% occupancy.

### Ligand Synthesis

### Synthesis of L

The ligand L was prepared as described previously.²⁸ However, procedures for the synthesis of the benzoylated thioamide (1) and the thioamide (2) were slightly modified.

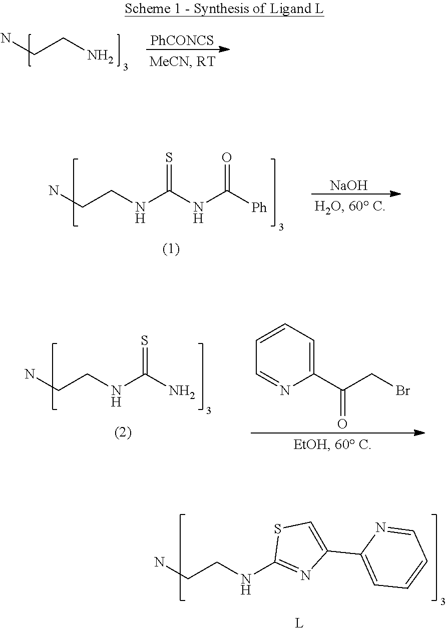

Synthesis of (1): To a solution of tris(2-aminoethyl)amine (1.0 g, 6.84 mmol) in acetone (50 mL) under an atmosphere of dinitrogen was added benzoyl isothiocyanate (3.7 g, 22.6 mmol) at such a rate to cause the reaction to gently reflux. After addition the reaction was stirred overnight during which time a colourless precipitate formed. The precipitate was isolated by filtration and washed with acetone (3×5 mL) giving (1) as a white solid. Yield=2.05 g (47%). ¹H NMR (400 MHz, DMSO-d⁶) δ (ppm) 11.20 (s, 3H, —NH), 11.0 (t, 3H, J=4.8, —CH₂Nh), 7.81 (d, 6H, J=7.2, Ph), 7.56 (t, 3H, J=7.4, Ph), 7.38 (t, 6H, J=7.6, Ph), 3.75 (q, 6H, J=5.7, —CH₂CH₂NH), 2.89 (t, 6H, J=6.0 Hz, —CH₂CH₂NH). ¹³C NMR [100 MHz, DMSO-d⁶]: δ_{C}=180.5 (C═S), 168.2 (C═O), 133.1 (CH), 132.7 (Q), 128.9 (CH), 128.6 (CH), 51.9 (CH₂), 42.6 (CH₂). ESI-MS m/z 636 (M+H⁺), HR ESI-MS found 636.1882 C₃₀H₃₃N₇S₃O₃ requires 636.1880 (error 0.46 ppm).

Synthesis of (2): The benzoylated urea derivative (1) (1.3 g, 2.05 mmol) was suspended in water (20 mL) and NaOH (820 mg, 20.5 mmol) added. The reaction was then heated to 60° C. and after 48 hrs the temperature was incrementally decreased allowing the solution to slowly cool to room temperature, avoiding formation on an oil and resulting in the formation of a colourless precipitate. Isolation by filtration and washing with ice cold water (2×1 mL) gave the tri-thiourea (2) as a colourless solid. Yield=503 mg (76%). ¹H NMR (400 MHz, DMSO-d⁶) δ (ppm) 7.55 (brs, 3H, —NH), 7.08 (brs, 6H, —NH₂), 3.44 (brs, 6H, —CH₂CH₂NH), 2.58 (brs, 6H, —CH₂CH₂NH). ¹³C NMR [100 MHz, DMSO-d⁶]: δ_{C}=183.5 (C═S), 52.9 (CH₂), 42.3 (CH₂). ESI-MS m/z 324 (M+H⁺), HR ESI-MS found 324.1089 C₉H₂₁N₇S₃ requires 324.1093 (error 1.03 ppm).

### Synthesis of L¹

Ligand L¹ (an analogue of ligand L) shown below was prepared according to the following protocol.

Synthesis of α-bromo-2-acetylisoquinoline: To a solution of 2-acetylisoquinoline (1.0 g, 6.28 mmol) in carbon tetrachloride (20 mL) at 80° C. was added liquid bromine (0.39 mL, 7.55 mmol) in carbon tetrachloride (0.6 mL) slowly and dropwise until TLC (SiO₂, 1% MeOH in DCM) showed the absence of the starting material. The reaction was cooled, diluted in dichloromethane (100 mL) and washed strenuously with saturated sodium carbonate solution (2×50 mL). The organic layer was removed, dried over anhydrous magnesium sulfate and solvents removed by reduced pressure to leave the crude product as a yellow oil. This was purified by column chromatography (1% MeOH in DCM, SiO₂) and solvents removed to leave the pure product as a slightly yellow oil (1.08 g, 72%). ¹H NMR (400 MHz, CDCl₃) δ (ppm) 9.06-9.00 (m, 1H), 8.62 (d, J=5.5 Hz, 1H), 7.90-7.95 (m, 2H), 7.81-7.73 (m, 2H), 5.04 (s, 2H). ¹³C NMR [100 MHz, CDCl₃]: δ_{C}=194.1 (Q), 150.1 (Q), 141.1 (CH), 137.1 (Q), 130.7 (CH), 129.7 (CH), 127.1 (CH), 126.53 (Q), 126.49 (CH), 125.6 (CH), 34.7 (CH₂). ESI-MS m/z 249.9864 (M+H⁺), observed neutral mass 248.9788, C₁₁H₈NOBr requires 248.9789 (error 0.37 ppm).

Synthesis of L¹: To a solution of α-bromo-2-acetylisoquinoline (500 mg, 2.10 mmol) in ethanol (20 mL), 1,1′, 1″-(nitrilotris(ethane-2,1-diyl))tris(thiourea) (226 mg, 0.70 mmol) was added. The reaction was then heated to 80° C. for 12 h, during which time all the solid dissolved and the solution turned yellow. The solution was then allowed to cool and the solvent removed under reduced pressure. The resultant oil was diluted in dichloromethane (50 mL) and washed with saturated aqueous sodium bicarbonate solution (2×25 mL). The combined organic layers were dried over anhydrous magnesium sulfate and solvent removed under reduced pressure to give the crude product as a brown/yellow oil. This was purified by column chromatography (SiO₂, 10% methanol in dichloromethane) to afford L¹ as an orange solid (277 mg, 51%). ¹H NMR (400 MHz, CDCl₃) δ (ppm) 8.88 (d, J=8.5, 3H), 8.56 (d, J=5.6, 3H), 7.81 (d, J=8.2, 3H), 7.64-7.57 (m, 6H), 7.48 (ddd, J=8.3, 7.1, 1.0, 3H), 7.09 (s, 3H), 6.39 (br s, 3H), 3.50 (q, J=5.3, 6H), 2.90 (t, J=5.56 Hz, 6H). ¹³C NMR [100 MHz, CDCl₃]: δ_{C}=169.2 (Q), 153.7 (Q), 150.9 (Q), 142.0 (CH), 137.1 (Q), 129.9 (CH), 127.9 (CH), 127.1 (CH), 126.72 (Q), 126.70 (CH), 120.5 (CH), 108.8 (CH), 53.3 (CH₂), 43.4 (CH₂). ESI-MS m/z 777.2363 (M+H⁺), observed neutral mass 776.2291, C₄₂H₃₆N₁₀S₃ requires 776.2287 (error 0.63 ppm).

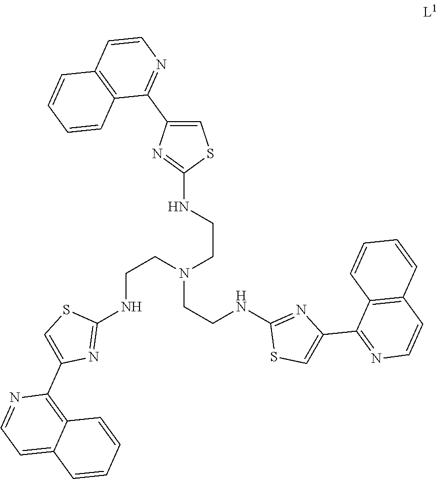 [SMILES: c1ccc2c(-c3csc(NCCN(CCNc4nc(-c5nccc6ccccc56)cs4)CCNc4nc(-c5nccc6ccccc56)cs4)n3)nccc2c1]

### Synthesis of Complexes

### Mononuclear Complexes

Synthesis of [LZn](ClO₄)₂. To a solution of Zn(ClO₄)₂·6H₂O (10 mg. 0.027 mmol) in MeCN (1 ml) was added a suspension of ligand L (12 mg, 0.019 mmol) in MeCN and the reaction sonicated until a clear solution had formed. To this was added water (˜1 ml) and the solution slowly allowed to evaporate, during which time pale yellow crystals were formed which were isolated by filtration and dried (yield=9 mg. 53%*). The [LMn](ClO₄)₂ complex was prepared in an analogous fashion using Mn(ClO₄)₂·6H₂O giving yellow crystals (yield=10 mg, 60%*). *percentage yield based on the moles of ligand used.

### Trinuclear Complexes

Synthesis of [L₂Zn₃(SO₄)](ClO₄)₄. To a solution of Zn(ClO₄)₂·6H₂O (10 mg. 0.027 mmol) in MeCN (1 ml) was added a suspension of ligand L (12 mg, 0.019 mmol) in MeCN and the reaction sonicated until a clear solution had formed. To this was added water (˜1 ml) containing Bu₄NHSO₄ (3.1 mg, 0.009 mmol) and the solution slowly allowed to evaporate during which time colourless crystals were formed which were isolated by filtration and dried (yield=11 mg, 60%). The [L₂Mn₃(SO₄)](ClO₄)₄ complex was prepared in an analogous fashion using Mn(ClO₄)₂·6H₂O giving yellow crystals (yield=9 mg, 50%).

Synthesis of [L₂Cu₃(O₃POPh)](ClO₄)₄. To a solution of Cu(ClO₄)₂·6H₂O (10 mg. 0.027 mmol) in MeCN (1 ml) was added a suspension of ligand L (12 mg, 0.019 mmol) in MeCN and the reaction sonicated until a clear solution had formed. To this was added water (˜1 ml) containing Na₂O₃POPh (2.3 mg, 0.009 mmol) and the solution slowly allowed to evaporate giving green crystals which were isolated by filtration and dried (yield=9 mg, 47%).

Synthesis of [L₂Zn₃(PO₄)](ClO₄)₃. To a solution of Zn(ClO₄)₂·6H₂O (10 mg. 0.027 mmol) in MeCN (1 ml) was added a suspension of ligand L (12 mg, 0.019 mmol) in MeCN and the reaction sonicated until a clear solution had formed. To this was added water (˜1 ml) containing Na₂O₃POPh (2.3 mg, 0.009 mmol) and the solution slowly allowed to evaporate giving yellow crystals which were isolated by filtration and dried (yield=10 mg, 54%). The [L₂Mn₃(PO₄)](ClO₄)₃ complex was prepared in an analogous fashion using Mn(ClO₄)₂·6H₂O giving yellow crystals (yield=9 mg, 50%).

### DISCUSSION

Reaction of 1.5 equivalents of L with either Zn(ClO₄)₂ or Mn(ClO₄)₂ results in a mononuclear complex (e.g. [LM]²⁺) as demonstrated by X-ray crystallography and ESI-MS. In the solid-state the zinc complex contains a 6-coordinate Zn²⁺ cation coordinated by six nitrogen atoms from three pyridyl-thiazole bidentate units from the same ligand. In the Mn²⁺ analogue the metal ion is again 6-coordinate, but this arises from coordination by four N-donor atoms from two bidentate pyridyl-thiazole units and two water O-donor atoms (FIG. 1). Both complexes differ from the Cu²⁺ derivative which can form the trimetallic capsule (e.g. [L₂Cu₃]⁶⁺) even in the presence of weakly interacting anions. The difference is attributed to the ability of Cu²⁺ to form 4-coordinate complexes (at least with pyridyl-thiazole donors) whereas both Zn²⁺ and Mn²⁺ prefer higher-coordinate geometry and without a strongly coordinating anion present a simple mononuclear species is formed. The coordination of water in the Mn²⁺ complex (e.g. [LMn(H₂O)₂]²⁺) is a consequence of the oxophillic nature of this hard cation and this behaviour is mirrored in all the structures with this metal. The formation of these species is also observed in the gas phase with ESI-MS studies showing that only ions corresponding to the mononuclear species are present.

Reaction of L with either Mn²⁺ or Zn²⁺ with (Bu₄N)HSO₄ (in the correct stoichiometric proportions) results in the formation of the capsule in which sulfate anions are encapsulated (e.g. [L₂M₃(SO₄)]⁴⁺) (FIG. 2). In the solid state the Zn²⁺ is isostructural to the Cu²⁺ derivative with a trinuclear [L₂Zn₃]⁶⁺ assembly and within it is an encapsulated sulfate anion (e.g. [L₃Zn(SO₄)]⁴⁺). Each of the three Zn²⁺ atoms are 5-coordinate arising from four N-donor atoms from two bidentate pyridyl-thiazole units and one oxygen donor from the sulfate anion. The sulfate is held within the capsule by three coordination bonds to Zn²⁺ supplemented by three —NH—O hydrogen bonding interactions. The remaining uncoordinated oxygen atom forms hydrogen bonds to three —NH donor units within the “upper rim” of the cavity. The Mn²⁺ forms a very similar type of assembly but each of the Mn²⁺ metal ions are 6-coordinate, which for one of the metal ions arises from four N-donor atoms from two bidentate pyridyl-thiazole units and two oxygen donors from the sulfate anion. The remaining two ions are also 6-coordinate and are coordinated by two bidentate N-donor ligands but are coordinated by one oxygen atom from the sulfate anion and one water molecule (e.g. [L₂Mn₃(H₂O)₂(SO₄)]⁴⁺). This demonstrates that whilst with weakly interacting anions (e.g. halides, ClO₄⁻, and BF₄⁻) both Zn²⁺ and Mn²⁺ form mononuclear complexes, with tetrahedral oxoanions these template the formation of the trimetallic capsule.

Ions in the ESI-MS at m/z 1844 and 1812 corresponding to {[L₂Zn₃(SO₄)(ClO₄)₃}⁺ and {[L₂Mn₃(SO₄)(ClO₄)₃}⁺ coupled with doubly charged ions indicate that these species are also observed in the gas phase.

Addition of disodium phenylphosphate to a solution of [L₂Cu₃]⁶⁺ in MeCN/H₂O results in a colour change from light blue to green. Crystals were then deposited after several days and analysis by X-ray crystallography shows that the trimetallic capsule is still formed but held inside the host is a PhOPO₃²⁻ anion. In a very similar fashion to the other oxoanions, PhOPO₃²⁻ is coordinated to the three Cu²⁺ metal ions supplemented by a series of —NH— anion interactions. However, due to the phenyl substituent the ligands adopt a slightly different conformation allowing the phenyl unit to occupy a cleft formed by two pyridyl-thiazole units (FIG. 3).

Reaction of two equivalents of L, three equivalents of either M(ClO₄)₂(where M=Zn²⁺ or Mn²⁺) and PhOPO₃Na₂ results in a very different species. In the solid-state both structures contain a central PO₄³⁻ anion held within the molecule by a series of interactions between the metal ions and amine hydrogen atoms (FIG. 4). The [L₂Mn₃(PO₄)]³⁺ complex is similar to the sulfate analogue and the three 6-coordinate Mn²⁺ metal ions are coordinated by two bidentate N-donor ligand domains but one metal ion is coordinated by two oxygen atoms from the anion and the remaining two metal ions are coordinated by one anion oxygen atom and a water molecule. The [L₂Zn₃(PO₄)]³⁺ is slightly different from the sulfate analogue and one metal ion is 6-coordinate arising from coordination of two bidentate N-donor ligand units and two oxygen atoms of the anion. The remaining two metal ions are only 5-coordinate as only one oxygen atom from the anion interacts with the metal.

This metal-dependent reactivity is also observed in the ESI-MS. Reaction of [L₂Cu₃](ClO₄)₆ with PhOPO₃Na₂ in water and MeCN gives ions at m/z 1914, 1024, 788 and 463 corresponding to {[L₂Cu₃(PhOPO₃)](ClO₄)₃}+, {[LCu₂(PhOPO₃)](ClO₄)}+, {[LCu](ClO₄)}+ and {[LCu₂(PhOPO₃)]}²⁺. Heating this sample at 80° C. shows no change in the ESI-MS spectrum indicating that the phenylphosphate dianion remains intact. A similar reaction of PhOPO₃Na₂ with Zn(ClO₄)₂ and L gave an ESI-MS with ions at m/z 1920 and 910 corresponding to {[L₂Zn₃(PhOPO₃)](ClO₄)₃}⁺ and {[L₂Zn₃(PhOPO₃)](ClO₄)₂}²⁺ respectively. Lower molecular weight ions at m/z 1029, 791 and 464 corresponding to {[LZn₂(PhOPO₃)](ClO₄)}⁺, {[LZn](ClO₄)}⁺ and {[LZn₂(PhOPO₃)]}²⁺ were also observed. However, heating this sample at 80° C. results in a dramatic change in the ESI-MS with the spectrum now much simplified with ions at m/z 1743 and 822 corresponding to {[L₂Zn₃(PO₄)](ClO₄)₂}⁺ and {[L₂Zn₃(PO₄)](ClO₄)}²⁺. This demonstrates that initially the Zn²⁺ containing complex reacts with phenyl phosphate dianion and in a similar fashion to the Cu²⁺ analogue and forms the trinuclear complex incorporating this anion (e.g. [L₂Zn₃(PhOPO₃)]⁴⁺). However, after either a few days at room temperature or heating at 80° C. for 1 hr the anion is hydrolysed and phosphate is encapsulated within the cryptand (see FIGS. 5a-5d). This hydrolysis is also confirmed by ¹H NMR as reaction of two equivalents of L, three equivalents of Zn(ClO₄)₂ and PhOPO₃Na₂ initially gives a broad complex spectrum but after 1 hr at 80° C. gave a spectrum that contains signals corresponding to [L₂Zn₃(PO₄)]³⁺ accompanied with signals corresponding to the phenol hydrolysis product. Reaction with Mn(ClO₄)₂ is similar to the Zn²⁺ analogue with ions in the ESI-MS corresponding to binding of phenyl phosphate observed initially (e.g. {[LMn₂(PhOPO₃)](ClO₄)}⁺) but ions corresponding to hydrolysis (e.g. {[L₂Mn₃(PO₄)](ClO₄)₂}⁺ and {[L₂Mn₃(PO₄)](ClO₄)}²⁺) are observed after heating for 1 hr.²⁹

The Zn²⁺ complex shows substrate specific differences in the rates of hydrolysis and its phosphatase activity. Analysis of the hydrolysis of phenyl phosphate dianion by the Zn²⁺ complex (in a 25%:75% mixture of DMSO and buffered H₂O solution (HEPES pH 7.5)) by ³¹P NMR shows a signal at −1.5 ppm corresponding to unhydrolysed PhOPO₃²⁻ at t=0 (FIG. 6, spectra B). After 19 hrs at 37° C. the major signal present is now observed at 8.6 ppm which is at an identical chemical shift to [L₂Zn₃(PO₄)]³⁺ and after 44 hrs virtually no signal corresponding to PhOPO₃²⁻ is observed (FIG. 6, spectra C, D). In a similar experiment using 4-nitrophenyl phosphate no 31P signals could be detected that corresponded to the starting material following mixing with the Zn²⁺ complex indicating almost immediate hydrolysis. Only [L₂Zn₃(PO₄)]³⁺ was observed, coupled with a rapid yellowing of the solution due to the formation of 4-nitrophenolate.

Given the importance of protein phosphorylation in inter- and intra-cellular signalling and to cell function, and its common dysregulation in cancers,^{30, 31} it was next analysed whether the Zn²⁺ complex could dephosphorylate the phosphorylated amino acids serine, threonine and tyrosine. Indeed, the Zn²⁺ complex resulted in dephosphorylation of serine-PO₃²⁻ and tyrosine-OPO₃²⁻ at similar hydrolysis rates to PhOPO₃²⁻ with substantial hydrolysis occurring over 24 hrs and completion after 48 hrs (FIG. 6, spectra E-H; M-P). Dephosphorylation of the amino acid threonine-OPO₃²⁻ by the Zn²⁺ complex was much slower however and after 48 hrs threonine-OPO₃²⁻ was still the major species (FIG. 6, spectra I-L). Differences in reactivity towards different substrates can be attributed to both steric and electronic effects. The difference in reactivity towards phenyl phosphate compared to the 4-nitro derivative is likely a consequence of the electron-withdrawing nitro group, which will enhance the hydrolysis. Serine-PO₃²⁻, tyrosine-OPO₃²⁻ and threonine-OPO₃²⁻ all have similar electronic properties but threonine has a methyl substituent close to the phosphorylated residue and it would seem likely this would result in unfavourable steric interactions upon binding of [L₂Zn₃]⁶⁺ as the —CHCH₃ unit would be housed deep in the cleft of the self-assembled species (see FIG. 3). It seems probable that this interaction would reduce the ability of the cryptand to bind the anion and hence reduce the hydrolysis rate. Both serine and tyrosine are less sterically demanding (tyrosine-OPO₃²⁻ is very similar to PhOPO₃²⁻ and serine-PO₃²⁻ has a less sterically demanding —CH₂— unit in this position) and consequently are hydrolysed more rapidly.

The rate of substrate hydrolysis is also dependent upon the metal used in the self-assembly process. The solid-state and ESI-MS data suggest that [L₂Cu₃]⁶⁺ does not hydrolyse phenyl phosphate but incorporates this anion within the assembly e.g. [L₂Cu₃(PhOPO₃)]⁴⁺. Comparison of the reactivity of the Zn²⁺ species verses the Mn²⁺ by monitoring the hydrolysis of 4-nitrophenyl phosphate by UV-Vis spectroscopy shows after 24 hrs the Mn²⁺ has hydrolysed with three times more phosphate, indicating that the Mn²⁺ is more active than the Zn²⁺ complex.

### Biological Studies

### Cell Lines and Culture

All cell lines used were maintained at low passage in antibiotic-free media and were obtained from ATCC (LGC Standards, Middlesex, UK) unless otherwise stated (Methods). HT29, DLD-1, HCT116 p53^{+/+} and HCT116 p53^{-/-} are all colorectal adenocarcinoma cell lines derived from different individuals and harbor different combinations of oncogenic lesions (except for the HCT116 isogenic cancer cell clones that are genetically identical except for p53 status). PSN-1, BxPC-3 and MiaPaCa2 are pancreatic carcinoma cell lines and A549 and H460 are lung carcinoma cell lines. HT29, DLD-1, PSN-1, BxPC-3, A549 and H460 cell lines were cultured in RPMI-1640 growth media (Sigma) containing 2 mM L-glutamine, 1 mM sodium pyruvate and 10% fetal bovine serum (FBS). HCT116 (p53^{+/+} and p53^{-/-}) and MiaPaCa2 cell lines were cultured in Dulbecco's Modified Eagle's Medium (Sigma), 2 mM L-glutamine and 10% FBS. The ARPE-19 retinal epithelial non-cancer cell line (Dunn et al., 1996) was cultured in DMEM/F12 media (Gibco), 2 mM L-glutamine, 1 mM sodium pyruvate and 10% FBS. The MCF10A non-cancerous human breast epithelial cell line was cultured in Minimal Essential Media Eagle (Sigma), 2 mM L-glutamine, 1 mM sodium pyruvate, 10% FBS and 1×non-essential amino acids (NEAA). NP1 and GBM1 cells were cultured on plasticware coated with poly-L-ornithine (5 μg/ml) and laminin (2.4 μg/ml) (Polson et al., 2018; Da Silva et al., 2019). NP1 cells were grown in DMEM/F12 media (Gibco) supplemented with 5% FBS, 20 ng/ml hFGF, 20 ng/ml rhEGF, 0.5×B-27 supplement (Gibco), 0.5×N-2 supplement (Gibco) and 1×GLUTAMAX (Gibco). GBM1 cells were cultured in Neurobasal media (Gibco) supplemented with 40 ng/ml hFGF, 40 ng/ml rhEGF, 0.5×B-27 supplement (Gibco) and 0.5×N-2 supplement (Gibco).

### Chemosensitivity Studies

[L₂M₃]⁶⁺ and all the other self-assembling complexes were freshly formed by adding DMSO to individual components mixing together by pipetting. These were then further diluted in cell culture media such that the final DMSO concentration that cells were exposed to was 0.2% (vehicle control). Cisplatin, oxaliplatin and carboplatin were dissolved in phosphate buffered saline. Cell lines were seeded into 96 well plates at 2×10³ cells per well and incubated overnight at 37° C. GBM1 cancer stem-like cells were seeded at 3×10³ cells per well and NP1 neural progenitors were seeded at 1.5×10³ cells per well. The following day, media was removed and replaced with fresh media containing test compounds at a range of concentrations. Cells were incubated with test compounds for a further 96 hours after which the media was removed and replaced with fresh media (200 μl/well). MTT was added (20 μl at 5 mg/ml) and cells were incubated for a further 4 hours. Media and MTT were removed and formazan crystals were dissolved in 150 μl of DMSO and the absorbance of the resulting solution determined at 540 nm. Dose response curves were constructed, and the concentration required to reduce cell growth by 50% (IC₅₀) determined. The selectivity index was defined as the IC₅₀ for non-cancer cells divided by the IC₅₀ for cancer cell lines with values >1 representing selectivity for cancer cells as opposed to non-cancer cells.

### Immunoblotting

Protein lysates and recombinant proteins were resolved on 15% SDS polyacrylamide gels. Proteins were electroblotted onto nitrocellulose membrane by wet transfer in 1×Tris Glycine buffer (Biorad) at 35 mA overnight at room temperature (Allison et al., 2014). After blocking of membranes, these were incubated with primary antibody overnight at 4° C. before addition of rabbit or mouse secondary antibody (HRP-conjugated) for 1 h at room temperature and development of blots by enhanced chemiluminescence. Primary antibodies were: anti-Src (total) (Cell Signalling Technology #2123 1:1000), anti-phosphorylated Src (Y527) (Cell Signalling Technology #21055 1:1000), anti-phosphorylated Src (Y416) (Cell Signalling Technology #69435 1:1000), anti-AMPKα (total) (Cell Signalling Technology #2532 1:1000), anti-phosphorylated AMPKα (T172) (Cell Signalling Technology #2532 1:1000), anti-phospho-Tyr (pan) (Cell Signalling Technology #8954 1:2000), anti-p53 (Santa Cruz, DO-1 clone, 1:1000), anti-β-actin (Merck MAB1501, 1:40,000). Quantification of signals was performed by densitometry using Image J software.

### Results and Discussion

[L₂Cu₃]⁶⁺ and [L₂Zn₃]⁶⁺ possess both potent and selective activity against most of the cancer cell lines tested compared to three non-cancer cell models utilised (FIG. 6, a-c). For [L₂Zn₃]⁶⁺, IC₅₀ values towards the cancer cell lines ranged from 70±13 nM against HCT116 p53^{-/-} and up to 59.07±5.60 μM against MiaPaCa2. IC₅₀ values for both complexes were mostly sub-μM towards cancer cells (HT-29, DLD-1, HCT116 (p53 wild type and null), BxPC3, A549 and H460 cell lines) with the exceptions being the pancreatic cancer cell line PSN1 and the GBM1 glioblastoma cancer stem cell model^{32,33} where IC₅₀ values were >1 μM. Cancer stem cells are typically chemoresistant,³⁴ however, importantly both complexes showed preferential activity towards the GBM1 cells compared to all three non-cancer cell models which included adult human brain progenitor cells (NP1).^{32,33} The MiaPaCa2 pancreatic cancer cell line was however inherently resistant to both complexes with IC₅₀ values >10 μM (FIG. 7a).

The magnitude of selectivity towards cancer cells was marked and for both [L₂Cu₃]⁶⁺ and [L₂Zn₃]⁶⁺ was over 10-fold for most of the cancer cell lines compared to all three non-cancer cell models (FIG. 7b,c). Remarkably, for [L₂Zn₃]⁶⁺ selectivity indices of over 2000 were obtained in the case of HCT116 p53^{-/-} cancer cells compared to ARPE-19 and MCF10A non-cancer cells (FIG. 7c). For the [L₂Cu₃]⁶⁺ complex, selectivity indices for many of the cancer cell lines were >100 with the [L₂Zn₃]⁶⁺ complex resulting in even higher SIs. Interestingly, however, whereas activity of the [L₂Cu₃]⁶⁺ complex was very similar against the three non-cancer cell models, the NP1 brain progenitor cells^{32,33} showed increased sensitivity to the Zn²⁺ complex.

Whilst the potency of both the Zn²⁺ and Cu²⁺ complexes (FIG. 7a) compares favourably with that of the clinically approved platinates (FIG. 7d), selectivity of the complexes is significantly superior under the same in vitro experimental conditions (FIG. 7b,c cf. 6e). In contrast, the Mn²⁺ complex, although a potent cytotoxin in vitro (FIG. 7d), it showed only modest preferential selectivity (˜2-fold) towards cancer cells which was comparable to that of the platinates (FIG. 7e).

The encapsulation of different specific anions (e.g. PO₄³⁻, SO₄²⁻ or PhOPO₃²⁻) into the [L₂Zn₃]⁶⁺ and [L₂Cu₃]⁶⁺ complexes at the point of self-assembly prior to any cell exposure impacts on both activity and selectivity. The effect is both anion and cell line dependent (see FIG. 8 and Further results section) with the inclusion of different anions having either minimal effect or causing an increase or decrease in potency depending on the cell line. Against the PSN1 cell line for example, both [L₂Zn₃(SO₄)]⁴⁺ and [L₂Zn₃(O₃POPh)]⁴⁺ are significantly more active than [L₂Zn₃(PO₄)]³⁺ or [L₂Zn₃]⁶⁺ and this translates into improved selectivity indices compared against the ARPE-19 cell line (FIG. 8). A similar but smaller increase in potency was also observed against HCT116 p53^{+/+} (but not the p53 null variant) and H460 cells treated with [L₂Zn₃(SO₄)]⁴⁺ and [L₂Zn₃(O₃POPh)]⁴⁺ leading to a corresponding increase in relative selectivity. In contrast, the inclusion of anions reduced the activity of [L₂Zn₃]⁶⁺ against HT-29, DLD-1, BxPC3 and A549 cells resulting in a corresponding reduction in relative selectivity (FIG. 8). Similar results were obtained with [L₂Cu₃]⁶⁺ complexes with PO₄³⁻, SO₄²⁻ or PhOPO₃²⁻ anions (see Further results section). The mechanistic basis for these differential effects requires further investigation. However, these results demonstrate that the activity of these complexes can be readily modulated or potentially ‘tuned’ towards different cancer cells by altering the metal and/or the anion providing an inherently flexible platform for drug discovery.

### Mechanistic Studies

Given the ability of the Zn²⁺ complex to dephosphorylate amino acids serine, tyrosine and threonine (FIG. 6) and the Cu²⁺ complex to bind phenyl phosphate (FIG. 3), it was examined whether the complexes affect the activity of kinases. This was assessed in a cell-free screen of 140 kinases using purified recombinant human kinases and substrate peptides incubated in the presence of ³³P ATP and 10 μM complex or solvent control. [L₂Zn₃]⁶⁺ and [L₂Cu₃]⁶⁺ inhibited the activity of multiple kinases to differing extents, with the most potently inhibited kinases being inhibited by near to 100% (FIG. 9a,b). [L₂Cu₃]⁶⁺ has inhibitory activity against a larger number of kinases than [L₂Zn₃]⁶⁺ which may reflect the fact that selectivity indices for [L₂Cu₃]⁶⁺ are comparatively lower than those for [L₂Zn₃]⁶⁺. In some cases, there is selective inhibition of specific kinases by either [L₂Zn₃]⁶⁺ or [L₂Cu₃]⁶⁺ (see Further results section). Across the kinome, the inhibition of kinases in the TK and CAMK family for [L₂Zn₃]⁶⁺ and the CAMK and AGC family for [L₂Cu₃]⁶⁺ were observed (FIG. 10e,f), however whether this reflects an overall preference of the compounds requires further investigation. For a few kinases, activity was increased by [L₂Cu₃]⁶⁺ or [L₂Zn₃]⁶⁺ (FIG. 9c,d), the most striking example being that of the proto-oncogene Src, a non-receptor tyrosine kinase.³⁵

There are several potential mechanisms which could lead the observed kinase inhibition ‘readout’ of this screen including, i) direct ATP hydrolysis by the complex(es) and differing Km of the kinases for ATP, and, ii) dephosphorylation of the peptide substrate by the phosphatase activity of the Zn²⁺ complex resulting in cycles of peptide phosphorylation by active recombinant enzyme and dephosphorylation by the complex. However, mass spectroscopy showed little or no hydrolysis of ATP when incubated with Zn²⁺ or Cu²⁺ complex alone (see Further results section). Similarly, neither complex resulted in dephosphorylation of a purified phosphorylated AMPK peptide (see Further results section) although AMPK is one of the most potently inhibited kinases by both complexes (FIG. 9a,b).

An alternative explanation of the observed effects which can be reconciled with both selective kinase inhibition and activation is that that the Zn²⁺ and Cu²⁺ complexes are modulating key regulatory phosphor-sites on the kinases themselves leading to enhanced or repressed kinase activity. In the case of the Zn²⁺ complex, dephosphorylation of specific phosphorylated regulatory amino acids through its phosphatase activity (FIG. 6) may lead to either kinase inhibition or activation. For the Cu²⁺ complex, it was hypothesised that it may bind to specific phosphorylated amino acids given its ability to bind but not hydrolyse phenyl phosphate (FIG. 3, see Further results section). This could affect kinase activity through influencing docking or binding of other proteins (eg. SH2-domain-containing activating or inhibitory proteins)³⁶ and through steric or structural effects.

To investigate these hypotheses further, regulatory phospho-amino acids of two key kinases identified by the kinase screen, AMPK and Src, were analysed following incubation of the recombinant kinases with Zn²⁺ or Cu²⁺ complexes. Phosphorylation of AMPKα at threonine 172 (p-T172) stimulates AMPK activity³⁷ and treatment of AMPK with either [L₂Zn₃]⁶⁺ or [L₂Cu₃]⁶⁺ significantly reduced p-T172 levels detectable by immunoblotting at a molecular weight of ˜63 kDa (FIG. 10).

These results are consistent with the inhibition of AMPK by both [L₂Zn₃]⁶⁺ and [L₂Cu₃]⁶⁺ observed in the kinase screen (FIG. 9a,b) but subtle differences in mechanisms are revealed. In the case of [L₂Cu₃]⁶⁺, high molecular weight bands of >250 kDa are detected with the p-T172 specific Src antibody suggesting that this compound is binding to the kinase, consistent with its ability to bind or encapsulate phosphoanionic molecules (eg. phenyl phosphate, FIG. 3). [L₂Zn₃]⁶⁺ on the other hand causes a clear reduction in p-T172 and no high molecular weight bands consistent with phosphatase activity towards this phosphorylated amino acid of recombinant AMKPα (FIG. 6).

For Src, effects of the complexes on two key regulatory phospho-amino acids were examined, Y527 and Y416 (FIG. 10). Phosphorylation at Y527 in the Src C-terminal domain is known to decrease Src activity whereas autophosphorylation at Y416 in the activation loop of the kinase domain increases Src activity.^{36,38} Here, incubation of recombinant Src with [L₂Zn₃]⁶⁺ resulted in a ˜30% decrease in p-Y527 relative to total Src levels and a ˜5-fold increase in p-Y416 levels (FIG. 10), both of which combined would be expected to result in enhanced Src kinase activity as was observed in the kinase screen (FIG. 9c,d). The decrease in p-Y527 is consistent with the selective phosphatase activity of [L₂Zn₃]⁶⁺. Y527 dephosphorylation reduces allosteric inhibition of Src kinase activity by the C-terminal domain, enabling autophosphorylation of Src at Y416.³⁵ Similar to AMPK, incubation of [L₂Cu₃]⁶⁺ with Src resulted in higher molecular weight bands detected with the total Src antibody which were not observed with control or [L₂Zn₃]⁶⁺ treatments, suggesting binding of [L₂Cu₃]⁶⁺ to Src. p-Y416 could not be detected raising the possibility that [L₂Cu₃]⁶⁺ binding to Src masks detection of this epitope, however, further studies are required to unravel the detailed mechanism as to how [L₂Cu₃]⁶⁺ binding to Src may increase its activity.

### Selective Induction of Autophagy and Cancer Cell ATP Depletion

Given the kinase screen results indicating that the [L₂Cu₃]⁶⁺ and [L₂Zn₃]⁶⁺ complexes can inhibit multiple kinases as well as activating several others (FIG. 9 and see Further results section) and the dysregulation of phospho-signalling in cancers,^{31, 39} this provides a likely mechanism by which they exert their cancer selective activity. Phenotypically, [L₂Zn₃]⁶⁺ induced the appearance of vacuoles in the HCT116 p53^{+/+} and p53^{-/-} cancer cells which were shown to be autophagic using the established autophagic tracer dye CYTO-ID.⁴⁰ The Mn²⁺ complex also induced autophagy whereas [L₂Cu₃]⁶⁺ did not (FIG. 11a, and see Further results section).

Autophagy is a catabolic process that is induced in response to metabolic stresses including low ATP levels and starvation.³⁷ It was hypothesised that the induction of autophagy by Zn²⁺ and Mn²⁺ complexes could be due to cellular ATP depletion resulting from their protein phosphatase activity and repeated futile cycles between protein phosphorylation by constitutively active oncogenic kinases³⁹ and dephosphorylation by the complexes. In support of this hypothesis, Zn²⁺ and Mn²⁺ complexes both caused a dose-dependent decrease in ATP levels (FIG. 11b and see ESI). This was much more pronounced in the HCT116 cancer cells than in ARPE19 non-cancerous cells with [L₂Zn₃]⁶⁺, indicating cancer selective ATP depletion correlating with the excellent cancer cell selectivity indices of the Zn²⁺ complex (FIG. 7c). There was less difference in the magnitude of ATP depletion between the HCT116 cancer cells and the ARPE19 non-cancer cells with Mn²⁺ complex treatment (see Further results section), consistent with its more modest selectivity indices (FIG. 7d). This may relate to more promiscuous phosphatase activity of the Mn²⁺ complex or its higher rates of hydrolysis (see Further results section).

Immunoblot analyses suggest that the autophagy is a compensatory catabolic response to sustain ATP levels and prevent bioenergetic failure and death with [L₂Zn₃]⁶⁺ inducing activation of the ‘low ATP’ sensing kinase AMPK³⁷ in HCT116 cancer cells but not in ARPE19 non-cancer cells (FIG. 12).

Thus, cellular levels of p-T172 of AMPKα were increased relative to total AMPKα levels specifically in the HCT116 cancer cells by [L₂Zn₃]⁶⁺ but not by [L₂Cu₃]⁶⁺ (FIG. 12). T172 AMPKα phosphorylation is induced by low cellular ATP levels and increased AMP or ADP levels, enabling competitive binding of AMP/ADP to the y regulatory subunit of AMPK enabling AMPKα phosphorylation by one of the AMPK upstream kinases and blocking phosphatase access to p-T172.³⁷ Thus, paradoxically, [L₂Zn₃]⁶⁺ causes dephosphorylation of T172 AMPKα and kinase inhibition in a cell-free system (ATP present) (FIG. 9a, 10) consistent with its phosphatase activity (FIG. 6) but results in increased cellular levels of p-T172 AMPK selectively in cancer cells (FIG. 12) which is attributed to selective ATP depletion (FIG. 11).

Following [L₂Zn₃]⁶⁺ treatment of HCT116 cancer cells, a small decrease (˜10%) in total levels of Tyr-phosphorylated proteins was detected by immunoblotting using a pan phospho-tyrosine antibody although there was also evidence of increased p-Y of some proteins (*) (FIG. 12). Importantly, both [L₂Cu₃]⁶⁺ and [L₂Zn₃]⁶⁺ treatment resulted in increased levels of the p53 tumour suppressor protein which is induced by many different types of cellular stress resulting in the co-ordination of an appropriate stress response which can include cell cycle arrest or cell death induction.⁴¹ Whilst p53 levels were increased by ˜1.7 fold by [L₂Cu₃]⁶⁺ and ˜2.7 fold by [L₂Zn₃]⁶⁺ treatment in the HCT116 cancer cells, levels of p53 actually decreased with treatment in the ARPE19 non-cancer cells further indicating differential effects of the complexes on cancer versus non-cancer cells (FIG. 12). The earlier chemosensitivity results indicate, however, that the cytotoxicity of the [L₂Cu₃]⁶⁺ and [L₂Zn₃]⁶⁺ is not dependent on p53; indeed both complexes were more active towards the HCT116 p53^{-/-} cells than the isogenic p53^{+/+} cells (FIG. 7a).

### Further Results

Chemosensitivity studies results: The effect of anions on the potency and selectivity of all test compounds evaluated is presented in FIGS. 13-15. The inclusion of anions either increased, decreased or had no effect on both potency (FIG. 13) and the selectivity (FIG. 14) of [L₂Cu₃]⁶⁺ and [L₂Zn₃]⁶⁺. Enhanced or reduced effects on potency and selectivity relative to [L₂Cu₃]⁶⁺ and [L₂Zn₃]⁶⁺ was strongly cell line dependent (FIG. 15). Particularly marked enhancement of both potency and selectivity (relative to ARPE-19) was observed for PSN1 and HCT116 p53^{+/+} cells treated with Cu²⁺ and Zn²⁺ ligands plus sulfate and phenyl phosphate anions. In contrast, the potency of the Cu²⁺ ligand plus the phenyl phosphate anion against BxPC3 cells is significantly reduced resulting in a reduction in selectivity compared to ARPE-19 cells. Different effects on non-cancer cell lines were also observed with only marginal effects of anion inclusion observed for the Cu²⁺ ligand (against both ARPE-19 and MCF10A cells) whereas for the Zn²⁺ ligand, its activity in the presence of all the anions tested was significantly reduced resulting in a relative loss of selectivity (FIG. 15). This suggests that the activity and selectivity of complexes can be tailored to individual cell lines, possibly via modulation of specific kinase inhibition activity and cell line dependent susceptibility to subsequent effects on the kinome.

Inhibition of kinase activity: The effect of [L₂Cu₃]⁶⁺ and [L₂Zn₃]⁶⁺ on the activity of 140 human recombinant kinases is presented in FIG. 16. Both compounds are multi-kinase inhibitors and visually, there are clear differences between the kinases that are inhibited by [L₂Cu₃]⁶⁺ and [L₂Zn₃]⁶⁺ with more [L₂Cu₃]⁶⁺ inhibiting more kinases than [L₂Zn₃]⁶⁺. The differences that exist indicate that a different spectrum of inhibitory activity exists with evidence of selectivity. Of interest, are the results demonstrating that the activity of some kinases is enhanced following drug treatment, particularly Src where significant stimulation of kinase activity was observed in these cell free assays.

Autophagy studies: The effects of 3.125 μM [L₂Cu₃]⁶⁺, [L₂Zn₃]⁶⁺ and [L₂Mn₃]⁶⁺ treatments (40 h) on the induction of cellular vacuoles and autophagy in the HCT116 p53^{-/-}, HCT116 p53^{+/+} and ARPE19 cells are presented in FIGS. 17-19 with representative cell images shown. At this concentration, induction of autophagy was most pronounced in the HCT116 p53^{-/-} cells with [L₂Mn₃]⁶⁺ treatment followed by [L₂Zn₃]⁶⁺ with no or little autophagy induced by [L₂Cu₃]⁶⁺ compared to vehicle control treated cells (FIG. 17). In the HCT116 p53^{+/+} cells, induction of autophagy was also evident in the [L₂Mn₃]⁶⁺ treated cells but was barely detectable in the [L₂Zn₃]⁶⁺ treated cells at this concentration compared to in the HCT116 p53^{-/-} cells (FIG. 18). This is consistent with [L₂Zn₃]⁶⁺ being ˜6-fold more active towards the HCT116 p53^{-/-} cells than the HCT116 p53^{+/+} cells based on 96 h IC₅₀ values. In the ARPE19 cells, levels of autophagy were low with all treatments and similar to basal levels in control-treated cells (FIG. 19).

Cellular ATP studies: The effects of 20 h treatment with a range of different concentrations of [L₂Cu₃]⁶⁺, [L₂Zn₃]⁶⁺ and [L₂Mn₃]⁶⁺ on total cellular ATP levels in HCT116 p53^{+/+} and ARPE19 cells are presented in FIG. 20. Differential effects are observed depending on the metal in the complex, with [L₂Mn₃]⁶⁺ having the most profound effects on both the HCT116 p53^{+/+} cancer and ARPE19 non-cancer cells. 6.25 μM [L₂Mn₃]⁶⁺ treatment for 20 h reduced ATP levels in the HCT116 p53^{+/+} cancer cells to <50% with ATP levels declining less in the ARPE19 non-cancer cells but reaching ˜40% with 100 μM [L₂Mn₃]⁶⁺ treatment. [L₂Zn₃]⁶⁺ also reduced ATP levels in the HCT116 p53^{+/+} cancer cells with levels reduced to <50% at concentrations of ≥25 μM [L₂Zn₃]⁶⁺ whereas in the ARPE19 cells ATP levels remained >70% with 100 μM [L₂Zn₃]⁶ treatment. Effects of [L₂Cu₃]⁶⁺ on ATP levels was much less, with ATP levels in the HCT116 cells reduced to <50% only at the highest concentration of 100 μM.

### SUMMARY

In summary, in this study three different metal-containing, self-assembled anion binding complexes are characterised and shown to have distinctive chemical and biological properties. Depending on the metal, the reactivity of the complexes towards different anionic species varied with the Zn²⁺ and Mn²⁺ complexes both showing significant phosphatase activity but with different rates of hydrolysis (Mn²⁺>>Zn²⁺) whereas the Cu²⁺ complex bound to, rather than hydrolysed, phospho-containing species. Remarkable selective activity towards particular cancer cells compared to non-cancer cells was shown by the Zn²⁺ and Cu²⁺ complexes by different mechanisms with the modulation of multiple kinases via either binding (Cu²⁺) or by de-phosphorylation (Zn²⁺) of regulatory sites on kinases. Zn²⁺ and Mn²⁺ complexes both induced cancer cell autophagy consistent with cellular ATP deficiency and bioenergetic failure as a result of their phosphatase activity and futile cycles of re-phosphorylation by oncogenic kinases. Further modulation of activity and selectivity profile by incorporation of different anions (eg. PO₄³⁻, SO₄²⁻ or PhOPO₃²⁻) pre-cell exposure indicates the ease of generating numeromodular' combinations of metal/anion binding self-assembling complexes that can differ in potency, selectivity and mechanism(s) of action towards disease.

### PART B

### Overview

[L₂Cu₃]⁶⁺ and [L₂Zn₃]⁶⁺ were evaluated at three different doses for in ovo anti-cancer activity, toxicity and anti-metastatic activity against tumors initiated from three different human cancer cell line models (H460, HT29, HCT116), in the chick embryo chorioallantoic membrane (CAM) assay.

### Methodology

Each compound was provided to the contract research organisation (CRO) Inovotion (La Tronche, France) conducting the in ovo study, as a powder pre-weighed in single vials (2 equivalents of ligand, 3 equivalents of metal; and stored at room temperature). Before use, this was then dissolved in DMSO to 50 mM to generate a stock solution and further freshly diluted in RPM11640 or McCoys aqueous cell culture media for administration.

A standard of care (SoC) drug for the particular tumour type was tested concurrently as a comparative control for in vivo efficacy and toxicity evaluation. A solvent control served as a negative control for both SoC and experimental compounds.

10⁶ tumor cells (H460, HT29 or HCT116) were grafted onto the upper chorioallantoic membrane (CAM) of the developing chick embryo on embryonic day 9 (E9). Access to the upper CAM was through a small hole drilled through the eggshell of fertilized White Leghorn eggs (into the air sac) on E9 (15 eggs per experimental group).

100 μl of freshly prepared experimental compound, vehicle control or standard-of-care (SoC) drug at their working concentrations were pipetted onto the tumor graft at E11, E13, E15 and E17. On day E18, the upper CAM (with tumor) was removed, washed by PBS buffer and then directly transferred in PFA (fixation for 48 hrs). After that, tumors were carefully cut away from normal CAM tissue and weighed.

Embryonic viability was checked daily. The number of dead embryos was also counted on E18, to evaluate treatment-induced embryo toxicity.

On day E18, a 1 cm² portion of the lower CAM was collected (n=8 per group), stored at −20° C. and later processed to extract DNA. Samples were analysed for human genomic DNA (qPCR for Alu sequences) as a quantitative marker of tumor metastasis to the lower CAM.

### Results

1. In Vivo Efficacy Against Tumors Initiated from Human Lung Cancer Cell Line H460

### Reduction of H460 Tumor Growth

Statistically significant regression of tumor growth (as determined by mean tumor weight at the end of the study, E18) was observed for [L₂Zn₃]⁶⁺ at all three tested doses and for [L₂Cu₃]⁶⁺ at the two highest tested doses (FIG. 21). Reduction of tumor growth for all three tested doses of [L₂Zn₃]⁶⁺ and the highest dose of [L₂Cu₃]⁶⁺ were comparable to that obtained with SoC.

### Analysis of Anti-Metastatic Activity

Statistically significant decreases in H460 metastatic load were observed for all three doses of [L₂Zn₃]⁶⁺ with reduction by the highest dose comparable to that observed with SoC (FIG. 22). However, there was no statistically significant metastatic regression with [L₂Cu₃]⁶⁺ at the three tested doses indicating the differential effects of [L₂Cu₃]⁶⁺ and [L₂Zn₃]⁶⁺.

### Analysis of Embryonic Toxicity

In term of toxicity, [L₂Cu₃]⁶⁺ and [L₂Zn₃]⁶⁺ were well tolerated at all doses tested with no statistically significant difference observed compared to the solvent (negative) control group (Table 1).

TABLE 1
Percentage of alive and dead embryos per experimental
group at the end of the H460 in ovo study.

| | Group | | | | | |
|---|---|---|---|---|---|---|
| Gr. # | Description | Total | Alive | Dead | % Alive | % Dead |
| 1 | Neg. Ctrl. | 14 | 12 | 2 | 85.71 | 14.29 |
| 2 | SoC | 14 | 12 | 2 | 85.71 | 14.29 |
| 3 | L2Zn3 [1] | 14 | 13 | 1 | 92.86 | 7.14 |
| 4 | L2Zn3 [2] | 13 | 10 | 3 | 76.92 | 23.08 |
| 5 | L2Zn3 [3] | 11 | 10 | 1 | 90.91 | 9.09 |
| 6 | L2Cu3 [1] | 14 | 12 | 2 | 85.71 | 14.29 |
| 7 | L2Cu3 [2] | 14 | 13 | 1 | 92.86 | 7.14 |
| 8 | L2Cu3 [3] | 14 | 12 | 2 | 85.71 | 14.29 |

### Summary (H460 Tumours)

Overall, these results show that both [L₂Cu₃]⁶⁺ and [L₂Zn₃]⁶⁺ are well tolerated at all tested doses with [L₂Zn₃]⁶⁺ shown to have comparable anti-cancer efficacy to the SoC in ovo, both in decreasing primary H460 tumor weight and metastatic burden. For [L₂Cu₃]⁶⁺, at the highest dose tested, comparable anti-cancer activity to the SoC was also observed in decreasing H460 tumor weight, however, no statistically significant metastatic regression was observed.

This provides the first evidence of in vivo efficacy of these compounds (at non-optimised doses) validating the anti-cancer activity and selectivity observed in vitro. The lack of embryonic toxicity observed suggests testing of higher doses is warranted (for potentially improved in vivo efficacy compared to the SoC).

2. In Vivo Efficacy Against Tumors Initiated from Human Colon Cancer Cell Line HT29

### Reduction of HT29 Tumor Growth

Statistically significant regression of tumor growth (as determined by mean tumor weight at the end of the study, E18) was observed for both [L₂Zn₃]⁶⁺ and [L₂Cu₃]⁶⁺ at their highest tested dose that was comparable that obtained with the SoC (FIG. 23).

### Analysis of Anti-Metastatic Activity

There was no reduction in HT29 tumor cell metastasis to the lower CAM either with the SoC or any of the tested doses of [L2Cu₃]⁶⁺ and [L₂Zn₃]⁶⁺ (data not shown).

### Analysis of Embryonic Toxicity

[L₂Cu₃]⁶⁺ and [L₂Zn₃]⁶⁺ were well tolerated in the chick embryos grafted with HT29 colon cancer cells with increasing dose escalation showing no increased embryonic toxicity (Table 2).

TABLE 2
Percentage of alive and dead embryos per experimental
group at the end of the HT29 in ovo study.

| | Group | | | | | |
|---|---|---|---|---|---|---|
| Gr. # | Description | Total | Alive | Dead | % Alive | % Dead |
| 1 | Neg. Ctrl. | 15 | 14 | 1 | 93.33 | 6.67 |
| 2 | SoC | 14 | 14 | 0 | 100 | 0 |
| 3 | L2Zn3 [1] | 14 | 12 | 2 | 85.71 | 14.29 |
| 4 | L2Zn3 [2] | 14 | 13 | 1 | 92.86 | 7.14 |
| 5 | L2Zn3 [3] | 14 | 13 | 1 | 92.86 | 7.14 |
| 6 | L2Cu3 [1] | 14 | 10 | 4 | 71.43 | 28.57 |
| 7 | L2Cu3 [2] | 14 | 12 | 2 | 85.71 | 14.29 |
| 8 | L2Cu3 [3] | 13 | 12 | 1 | 92.31 | 7.69 |

### Summary (HT29 Tumours)

Overall, these results show that both [L₂Cu₃]⁶⁺ and [L₂Zn₃]⁶⁺ are well tolerated at all tested doses with both [L₂Cu₃]⁶⁺ and [L₂Zn₃]⁶⁺ at the highest dose tested shown to decrease tumor weight to a similar level to that seen with the SoC. This provides further evidence of in vivo efficacy of these compounds (at non-optimised doses) against a second tumour type (colorectal cancer) and further validates the anti-cancer activity and selectivity observed in vitro. The lack of embryonic toxicity observed suggests testing of higher doses is warranted (for potentially improved in vivo efficacy compared to the SoC and anti-metastatic activity).

3. In Vivo Efficacy Against Tumors Initiated from Human Colon Cancer Cell Line HCT116

### Reduction of HCT116 Tumor Growth

Statistically significant regression of HCT116 tumor growth (as determined by mean tumor weight at the end of the study, E18) was observed for [L₂Zn₃]⁶⁺ at the highest two doses tested and for [L₂Cu₃]⁶⁺ at all three tested doses, although effects were not as pronounced as obtained with the SoC (FIG. 24).

### Analysis of Anti-Metastatic Activity

Decreases in HCT116 metastatic load were observed for all three doses of [L₂Zn₃]⁶⁺ comparable to that observed with SoC (FIG. 25).

### Analysis of Embryonic Toxicity

In term of embryo toxicity, L2Zn3 and L2Cu3 were well tolerated at all doses tested with no statistically significant difference observed compared to the solvent (negative) control group (Table 3).

TABLE 3
Percentage of alive and dead embryos per experimental
group at the end of the HCT116 in ovo study.

| | Group | | | | | |
|---|---|---|---|---|---|---|
| Gr. # | Description | Total | Alive | Dead | % Alive | % Dead |
| 1 | Neg. Ctrl. | 17 | 15 | 2 | 88.24 | 11.76 |
| 2 | SoC | 15 | 14 | 1 | 93.33 | 6.67 |
| 3 | L2Zn3 [1] | 15 | 15 | 0 | 100 | 0 |
| 4 | L2Zn3 [2] | 15 | 13 | 2 | 86.67 | 13.33 |
| 5 | L2Zn3 [3] | 15 | 12 | 3 | 80.00 | 20.00 |
| 6 | L2Cu3 [1] | 14 | 14 | 0 | 100 | 0 |
| 7 | L2Cu3 [2] | 15 | 13 | 2 | 86.67 | 13.33 |
| 8 | L2Cu3 [3] | 15 | 13 | 2 | 86.67 | 13.33 |

### Summary (HCT116 Tumours)

These results show that both [L₂Cu₃]⁶⁺ and [L₂Zn₃]⁶⁺ are well tolerated at all tested doses with [L₂Zn₃]⁶⁺ at the highest two tested doses and for [L₂Cu₃]⁶⁺ at all three doses resulting in statistically significant regression of tumor growth. In term of metastatic invasion, none of the conditions induce a significant regression of the metastatic load. However a strong regression tendency was observed for [L₂Zn₃]⁶⁺ (all doses) similar to that obtained with the SoC. This provides further evidence of in vivo efficacy of these compounds (at non-optimised doses). The lack of embryonic toxicity observed suggests testing of higher doses is warranted (for potentially improved in vivo efficacy and anti-metastatic activity compared to the SoC).

### CONCLUSIONS

[L₂Cu₃]⁶⁺ and [L₂Zn₃]⁶⁺ were both tested at three non-optimised doses (based on their in vitro IC₅₀ values) for in vivo anti-cancer activity against tumors initiated from three different human cancer cell line models (H460, HT29, HCT116) in the chick embryo chorioallantoic membrane (CAM) assay. Against each tumour model, for both [L₂Cu₃]⁶⁺ and [L₂Zn₃]⁶⁺ statistically significant regression of tumour growth was observed, with effects most pronounced against the H460 lung cancer model (FIG. 21) and comparable to SoC for [L₂Zn₃]⁶⁺ in both H460 and HT29 models (FIGS. 21, 23). Anti-metastatic effects were also observed for [L₂Zn₃]⁶⁺ comparable to that obtained with the SoC in both H460 and HCT116 models (FIGS. 22, 25). Whilst these results provide initial proof-of-concept of in vivo anti-cancer efficacy with three different models to differing extents (most notably against the H460 lung cancer xenograft), it is also noteworthy that both [L₂Cu₃]⁶⁺ and [L₂Zn₃]⁶⁺ were extremely well tolerated (no statistically significant embryonic lethality) at the tested doses indicating scope for further dose escalation and optimisation.

While specific embodiments of the invention have been described herein for the purpose of reference and illustration, various modifications will be apparent to a person skilled in the art without departing from the scope of the invention as defined by the appended claims.

### REFERENCES

¹ Matsumoto, Y. et al. A landmark in drug discovery based on complex natural product synthesis. Sci Rep. 9, 8656 (2019).
² Lebn, J. “Supramolecular Chemistry”. Science. 260, 1762-23 (1993).
³ Lehn, J. M. “Perspectives in Supramolecular Chemistry—From Molecular Recognition towards Molecular Information Processing and Self-Organization”. Angew Chem Int Ed. 29, 1304-1319 (1990).
⁴ Lehn, J.-M., Supramolecular Chemistry, VCH, Weinheim (1995).
⁵ Steed, J. W., Atwood, J. L., Supramolecular Chemistry, John Wiley and Sons, Chichester (2000)
⁶ Hannon, M. J. & Childs, L. J. Helices and helicates: beautiful supramolecular motifs with emerging applications. Supramol. Chem. 16, 7-22 (2004).
⁷ Albrecht, M. “Let's twist again” double-stranded, triple-stranded, and circular helicates. Chem. Rev. 101, 3547-3498 (2001)
⁸ Albrecht, M. Dicatechol ligands: novel building-blocks for metallo-supramolecular chemistry. Chem. Soc. Rev. 27, 281-288 (1998).
⁹ Piguet, C., Bernardinelli, G. & Hopfgartner, G. Helicates as versatile supramolecular complexes. Chem. Rev. 97, 2005-2062 (1997).
¹⁰ Ahmedova, A. Biomedical applications of metallosupramolecular assemblies—structural aspects of the anticancer activity. Front. Chem. 6, 620 (2018).
¹¹ Sepehrpour, H., Wenxin, F., Sun, F. Y., Stang. P. J. Biomedically Relevant Self-Assembled Metallacycles and Metallacages. J. Am. Chem. Soc. 141, 14005-14020 (2019).
¹² Cook, T. R., Vajpayee, V., Lee, M. H., Stang, P. J. & Chi, K.-W. Biomedical and biochemical applications of self-assembled metallacycles and metallacages. Accounts of Chem. Res. 46, 2464-2474 (2013).
¹³ Kaner, R. A. & Scott, P. Metallohelices: potential mimetics of α-helical peptides in cancer treatment? Future Med. Chem. 7, 1-4 (2015).
¹⁴ Bruijnincx, P. C. A & Sadler, P. J. New trends for metal complexes with anticancer activity. Current Opinion in Chemical Biology. 12, 197-206 (2008).
¹⁵ Hannon, M. J., Moreno, V., Prieto, M. J., Moldrheim, E., Sletten, E., Meistermann, I., Isaac, C. J., Sanders, K. J. & Rodger, A. Intramolecular DNA coiling mediated by a metallo-supramolecular cylinder. Angew. Chem. Int. Ed. 40, 880-884 (2001).
¹⁶ Brabec, V., Howson, S. E., Kaner, R. A., Lord, R. M., Malina, J., Phillips, R. M., Abdallah, Q. M. A., McGowan, P. C., Rodger, A. & Scott, P. Metallohelices with activity against cisplatin-resistant cancer cells; does the mechanism involve DNA binding? Chem. Sci. 4, 4407-4416 (2013).
¹⁷ Richards, A. D., Rodger, A., Hannon, M. J. & Bolhuis, A. Antimicrobial activity of an iron triple helicate. Int. J. of Antimicro. Agents. 33, 469-472 (2009).
¹⁸ Faulkner, A. D., Kaner, R. A., Abdallah, Q. M. A., Clarkson, G., Fox, D. J., Gurnani, P., Howson, S. E., Phillips, R. M., Roper, D. I., Simpson, D. H. & Scott, P. Asymmetric triplex metallohelices with high and selective activity against cancer cells. Nature Chemistry. 6, 797-803 (2014).
¹⁹ Allison, S. J. et al. Ruthenium-containing linear helicates and mesocates with tuneable p53-selective cytotoxicity in colorectal cancer cells. Angew. Chem. Int. Ed. 57 9799-9804 (2018).
²⁰ Han, J., et al. Bioconjugation strategies to couple supramolecular exo-functionalized palladium cages to peptides for biomedical applications. Chem. Commun. 53, 1405-1408 (2017).
²¹ Lewis, J. E. M., Gavey, E. L., Cameron, S. A., Crowley, J. D. Stimuli-responsive Pd₂L₄ metallosupramolecular cages: towards targeted cisplatin drug delivery. Chem. Sci. 3, 778-784 (2012).
²² Wurdak, H. et al. An RNAi screen identifies TRRAP as a regulator of brain tumor-initiating cell differentiation. Cell Stem Cell. 6, 27-47 (2010).
²³ Polson, E. S et al. KHS101 disrupts energy metabolism in human glioblastoma cells and reduces tumor growth in mice. Sci Transl Med. 10, eaar2718 (2018).
²⁴ Dunn, K. C., Aotaki-Keen, A. E., Putkey, F. R., Hjelmeland, L. M. ARPE-19, a human retinal pigment epithelial cell line with differentiated properties. Exp. Eye Res. 62, 155-169 (1996).
²⁵ Soule, H. D. et al. Isolation and characterization of a spontaneously immortalized human breast epithelial cell line, MCF-10. Cancer Res. 50, 6075-86 (1990).
²⁶ SHELXTL Program System, Vers. 5.1, Bruker Analytical X-ray Instruments Inc., Madison, W I, 1998.
²⁷ G. M. Sheldrick, SADABS: A Program for Absorption Correction with the Siemens SMART System, University of Göttingen (Germany), 1996.
²⁸ C. R. Rice, C. Slater, R. A. Faulkner, R. L. Allan, Angew. Chem. 2018, 57, 13255-13259.
²⁹ Cao, R., MUller, P., Lippard, S. J. Tripodal tris-tacn and tris-dpa platforms for assembling phosphate-templated trimetallic centers. J. Am. Chem. Soc. 132, 17366-17369 (2010)
³⁰ Hunter, T. Protein kinases and phosphatases: the yin and yang of protein phosphorylation and signaling. Cell 80, 225-236 (1995).
³¹ Cohen, P. Protein kinases—the major drug targets of the twenty-first century? Nat Rev Drug Discov 1, 309-315 (2002).
³² Wurdak, H. et al. An RNAi screen identifies TRRAP as a regulator of brain tumor-initiating cell differentiation. Cell Stem Cell. 6, 27-47 (2010).
³³ Polson, E. S et al. KHS101 disrupts energy metabolism in human glioblastoma cells and reduces tumor growth in mice. Sci Transl Med. 10, eaar2718 (2018).
³⁴ Zhao, J. Cancer stem cells and chemoresistance: the smartest survive the raid. Pharmacol Therapeut 160, 145-58 (2016).
³⁵ Thomas, S. M., Brugge J. S. Cellular functions regulated by Src family kinases. Annu Rev Cell Dev Biol 13, 513-609 (1997).
³⁶ Pawson, T. Specificity in signal transduction: from phosphotyrosine-SH2 domain interactions to complex cellular systems. Cell 116, 191-203 (2004).
³⁷ Hardie, D. G., Ross F. A, Hawley, S. A. AMPK: a nutrient and energy sensor that maintains energy homeostasis. Nat. Rev. Mol. Cell. Biol. 13, 251-262 (2012).
³⁸ Hunter, T. A tail of two src's: mutatis mutandis Cell 49, 1-4 (1987).
³⁹ Blume-Jensen, P., Hunter, T. Oncogenic kinase signalling. Nature 411, 355-65 (2001).
⁴⁰ Chan, L. L.-Y. et al. A novel image-based cytometry method for autophagy detection in living cells. Autophagy 8, 1371-82 (2012).
⁴¹ Horn, H. F., Vousden V. H. Coping with stress: multiple ways to activate p53. Oncogene 26, 1306-16 (2007).

1. A compound having a structure according to Formula I shown below, or a pharmaceutically acceptable salt, hydrate or solvate thereof, for use as a medicament: 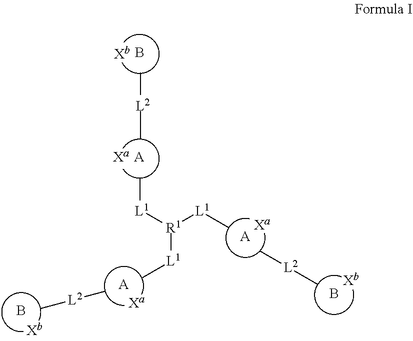
wherein
R¹ is selected from the group consisting of N, CR², aryl, heteroaryl, carbocyclyl and heterocyclyl, where any aryl, heteroaryl, carbocyclyl or heterocyclyl in R¹ is optionally substituted with one or more R³;
each R³ is independently selected from the group consisting of hydroxy, cyano, halogen, (1-4C)alkyl, (1-4C)haloalkyl, (2-4C)alkenyl, (2-4C)alkynyl, aryl, aryl(1-3C)alkyl, heteroaryl, heteroaryl(1-3C)alkyl, carbocyclyl, carbocyclyl(1-3C)alkyl, heterocyclyl, heterocyclyl(1-3C)alkyl, —OR^{3a}, —NR^{3a}R^{3b}, —C(O)—R^{3a}, —C(O)—OR^{3a}, —O—C(O)—R^{3a}, —C(O)—NR^{3a}R^{3b}, —N(R^{3a})C(O)—R^{3b} and —S(O)₀₋₂R^{3a}, where any (1-4C)alkyl, (1-4C)haloalkyl, (2-4C)alkenyl, (2-4C)alkynyl, aryl, aryl(1-3C)alkyl, heteroaryl, heteroaryl(1-3C)alkyl, carbocyclyl, carbocyclyl(1-3C)alkyl, heterocyclyl or heterocyclyl(1-3C)alkyl in R³ is optionally substituted with one or more R^{3c};
R^{3a} and R^{3b} are each independently selected from the group consisting of hydrogen, (1-3C)alkyl and (1-3C)haloalkyl;
each R^{3c} is independently selected from the group consisting of hydroxy, halogen, cyano, amino, (1-3C)alkyl, (1-3C)alkoxy and (1-3C)haloalkyl;
R² is selected from the group consisting of hydrogen, hydroxy, cyano, halogen, (1-4C)alkyl, (1-4C)haloalkyl, (2-4C)alkenyl, (2-4C)alkynyl, aryl, aryl(1-3C)alkyl, heteroaryl, heteroaryl(1-3C)alkyl, carbocyclyl, carbocyclyl(1-3C)alkyl, heterocyclyl, heterocyclyl(1-3C)alkyl, —OR^{2a}, —NR^{2a}R^{2b}, —C(O)—R^{2a}, —C(O)—OR^{2a}, —O—C(O)—R^{2a}, —C(O)—NR^{2a}R^{2b}, —N(R^{2a})C(O)—R^{2b} and —S(O)₀₋₂R^{9a}, where any (1-4C)alkyl, (1-4C)haloalkyl, (2-4C)alkenyl, (2-4C)alkynyl, aryl, aryl(1-3C)alkyl, heteroaryl, heteroaryl(1-3C)alkyl, carbocyclyl, carbocyclyl(1-3C)alkyl, heterocyclyl or heterocyclyl(1-3C)alkyl in R² is optionally substituted with one or more R^{2c};
R^{2a} and R^{2b} are each independently selected from the group consisting of hydrogen, (1-3C)alkyl and (1-3C)haloalkyl;
each R^{2c} is independently selected from the group consisting of hydroxy, halogen, cyano, amino, (1-3C)alkyl, (1-3C)alkoxy and (1-3C)haloalkyl;
each L¹ is a group:
—(W)ₙ—(X)ₘ—(Y)ₒ—(Z)ₚ—
in which
n and o are each independently 0, 1 or 2, and m and p are each independently 0 or 1, with the provisos that when m and p are both 1, o is not 0;
each W is selected from the group consisting of (1-3C)alkylene, (2-3C)alkenylene, (2-3C)alkynylene, arylene, heteroarylene, carbocyclylene and heterocyclylene, where any (1-3C)alkylene, (2-3C)alkenylene, (2-3C)alkynylene, arylene, heteroarylene, carbocyclylene or heterocyclylene in W is optionally substituted with one or more W^{a}, where each W^{a} is independently selected from the group consisting of hydroxy, cyano, halogen, amino, (1-2)alkoxy and (1-2C)haloalkyl;
X is selected from the group consisting of —O—, —C(O)—, —C(O)—O—, —O—C(O)—, —S(O)₀₋₂—, —C(O)—N(R^{x})—, —N(R^{x})—C(O)—, —NR^{x}—, —N(R^{x})—C(O)—NR^{x}—, —SO₂N(R^{x})—, and —N(R^{x})SO₂, where each R^{x} is independently selected from the group consisting of hydrogen, hydroxy, cyano, (1-4C)alkyl, (2-4C)alkenyl and (2-4C)alkynyl;
each Y is selected from the group consisting of (1-3C)alkylene, (2-3C)alkenylene, (2-3C)alkynylene, arylene, heteroarylene, carbocyclylene and heterocyclylene, where any (1-3C)alkylene, (2-3C)alkenylene, (2-3C)alkynylene, arylene, heteroarylene, carbocyclylene or heterocyclylene in Y is optionally substituted with one or more Y^{a}, where each Y^{a} is independently selected from the group consisting of hydroxy, cyano, halogen, amino, (1-2)alkoxy and (1-2C)haloalkyl;
Z is selected from the group consisting of —O—, —C(O)—, —C(O)—O—, —O—C(O)—, —S(O)₀₋₂—, —C(O)—N(R^{z})—, —N(R^{z})—C(O)—, —NR^{z}—, —N(R^{z})—C(O)—NR^{z}—, —SO₂N(R^{z})—, and —N(R^{z})SO₂, where each R^{z} is independently selected from the group consisting of hydrogen, hydroxy, cyano, (1-4C)alkyl, (2-4C)alkenyl and (2-4C)alkynyl;
X^{a} is a ring heteroatom located within ring A and is selected from N and O;
each ring A is a monocyclic heteroaryl, bicyclic heteroaryl, monocyclic heterocycle or bicyclic heterocycle, any one of which is optionally substituted with one or more R⁴, where each R⁴ is independently selected from the group consisting of hydroxy, cyano, halogen, (1-6C)alkyl, (1-6C)haloalkyl, (2-6C)alkenyl, (2-6C)alkynyl, aryl, aryl(1-3C)alkyl, heteroaryl, heteroaryl(1-3C)alkyl, carbocyclyl, carbocyclyl(1-3C)alkyl, heterocyclyl, heterocyclyl(1-3C)alkyl, —R^{4a}—OR^{4b}, —R^{4a}—NR^{4b}R^{4c}, —R^{4a}—C(O)—R^{4b}, —R^{4a}—C(O)—OR^{4b}, —R^{4a}—O—C(O)—R^{4b}, —R^{4a}—C(O)—NR^{4b}R^{4c}, —R^{4a}—N(R^{4b})C(O)—R^{4c} and —R^{4a}—S(O)₀₋₂R^{4b}, where any (1-6C)alkyl, (1-6C)haloalkyl, (2-6C)alkenyl, (2-6C)alkynyl, aryl, aryl(1-3C)alkyl, heteroaryl, heteroaryl(1-3C)alkyl, carbocyclyl, carbocyclyl(1-3C)alkyl, heterocyclyl or heterocyclyl(1-3C)alkyl in R⁴ is optionally substituted with one or more R^{4d};
R^{4a} is absent or is (1-3C)alkylene that is optionally substituted with one or substituents selected from group consisting of hydroxy, halo and amino;
R^{4b} and R^{4c} are each independently selected from the group consisting of hydrogen, (1-3C)alkyl and (1-3C)haloalkyl;
each R^{4d} is independently selected from the group consisting of hydroxy, halogen, cyano, amino, (1-3C)alkyl, (1-3C)alkoxy and (1-3C)haloalkyl;
X^{b} is a ring heteroatom located within ring B and is selected from N and O
each ring B is a monocyclic heteroaryl, bicyclic heteroaryl, monocyclic heterocycle or bicyclic heterocycle, any one of which is optionally substituted with one or more R⁵, where each R⁵ is independently selected from the group consisting of hydroxy, cyano, halogen, (1-6C)alkyl, (1-6C)haloalkyl, (2-6C)alkenyl, (2-6C)alkynyl, aryl, aryl(1-3C)alkyl, heteroaryl, heteroaryl(1-3C)alkyl, carbocyclyl, carbocyclyl(1-3C)alkyl, heterocyclyl, heterocyclyl(1-3C)alkyl, —R^{5a}—OR^{5b}, —R^{5a}—NR^{5b}R^{5c}, —R^{5a}—C(O)—R^{5b}, —R^{5a}—C(O)—OR^{5b}, —R^{5a}—O—C(O)—R^{5b}, —R^{5a}—C(O)—NR^{5b}R^{5c}, —R^{5a}—N(R^{5b})C(O)—R^{5c} and —R^{5a}—S(O)₀₋₂R^{5b}, where any (1-6C)alkyl, (1-6C)haloalkyl, (2-6C)alkenyl, (2-6C)alkynyl, aryl, aryl(1-3C)alkyl, heteroaryl, heteroaryl(1-3C)alkyl, carbocyclyl, carbocyclyl(1-3C)alkyl, heterocyclyl or heterocyclyl(1-3C)alkyl in R⁵ is optionally substituted with one or more R^{5d};
R^{5a} is absent or is (1-3C)alkylene that is optionally substituted with one or substituents selected from group consisting of hydroxy, halo and amino;
R^{5b} and R^{5c} are each independently selected from the group consisting of hydrogen, (1-5C)alkyl (e.g. (1-3C)alkyl) and (1-3C)haloalkyl;
each R^{5d} is independently selected from the group consisting of hydroxy, halogen, cyano, amino, (1-3C)alkyl, (1-3C)alkoxy and (1-3C)haloalkyl; and
each L² is selected from the group consisting of absent (in which case ring A is bonded directly to ring B), (1-2C)alkylene, ethenylene and ethynylene, where any (1-2C)alkylene, ethenylene and ethynylene in L² is optionally substituted with one or more substituents selected form the group consisting of hydroxy, halogen, cyano, amino, (1-3C)alkyl, (1-3C)alkoxy and (1-3C)haloalkyl;
and wherein the compound of Formula I, or the pharmaceutically acceptable salt, hydrate or solvate thereof, is for use in combination with a source of M, wherein M is selected from the group consisting of Zn²⁺, Mn²⁺, Cu²⁺, Fe²⁺, CO²⁺ and Ni²⁺.

2. The compound, pharmaceutically acceptable salt, hydrate or solvate for use according to claim 1, wherein each ring A is a 5-6 membered monocyclic heteroaryl containing 1, 2 or 3 ring heteroatoms in total independently selected from N, O and S, or a 5-6 membered monocyclic heterocycle containing 1, 2 or 3 ring heteroatoms in total independently selected from N, O and S, wherein each ring A is optionally substituted with one or more R⁴, and
X^{a} is located immediately adjacent the carbon atom bonded to L¹;

3. The compound, pharmaceutically acceptable salt, hydrate or solvate for use according to claim 1 or 2, wherein each ring B is:
i) a 5-6 membered monocyclic heterocycle containing 1, 2 or 3 ring heteroatoms in total that are independently selected from N, O and S;
ii) a 5-6 membered monocyclic heteroaryl containing 1, 2 or 3 ring heteroatoms in total that are independently selected from N, O and S;
iii) a 9-10 membered bicyclic heterocycle containing 1, 2 or 3 ring heteroatoms in total that are independently selected from N, O and S; or
iv) a 9-10 membered bicyclic heteroaryl containing 1, 2 or 3 ring heteroatoms in total that are independently selected from N, O and S,
wherein any ring in B is optionally substituted with one or more R⁵, and
X^{b} is located immediately adjacent the carbon atom bonded to L².

4. The compound, pharmaceutically acceptable salt, hydrate or solvate for use according to claim 1, 2 or 3, wherein each ring A is group: 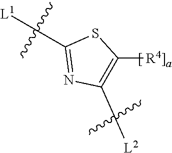
wherein a is 0 or 1.

5. The compound, pharmaceutically acceptable salt, hydrate or solvate for use according to any preceding claim, wherein each ring B is any of the following: 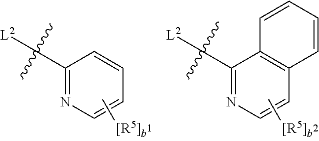
wherein:
b¹ is 0, 1 or 2, and
b² is 0, 1, 2 or 3.

6. The compound, pharmaceutically acceptable salt, hydrate or solvate for use according to any preceding claim, wherein
each W is selected from the group consisting of (1-3C)alkylene or phenylene, where any (1-3C)alkylene or phenylene in W is optionally substituted with one or more W^{a};
X is selected from the group consisting of —C(O)—N(R^{x})—, —N(R^{x})—C(O)— and —NR^{x}—;
each Y is selected from the group consisting of (1-3C)alkylene or phenylene, where any (1-3C)alkylene or phenylene in Y is optionally substituted with one or more Y^{a}; and
Z is selected from the group consisting of —C(O)—N(R^{z})—, —N(R^{z})—C(O)— and —NR^{z}—.

7. The compound, pharmaceutically acceptable salt, hydrate or solvate for use according to any preceding claim, wherein:
n is 0 or 1 and W is selected from the group consisting of (1-3C)alkylene or phenylene,
where any (1-3C)alkylene or phenylene in W is optionally substituted with one or more W^{a}, where each W^{a} is independently selected from the group consisting of hydroxy, halogen, (1-2)alkoxy and (1-2C)haloalkyl;
m is 0;
o is 0 or 1 and Y is selected from the group consisting of (1-3C)alkylene or phenylene, where any (1-3C)alkylene or phenylene in Y is optionally substituted with one or more Y^{a}, where each Y^{a} is independently selected from the group consisting of hydroxy, halogen, (1-2)alkoxy and (1-2C)haloalkyl; and
p is 1 and Z is —NR^{z}—, where R^{z} is hydrogen.

8. The compound, pharmaceutically acceptable salt, hydrate or solvate for use according to any preceding claim, wherein:
n is 0 or 1 and W is selected from the group consisting of (1-3C)alkylene or phenylene, where any (1-3C)alkylene or phenylene in W is optionally substituted with one or more W^{a}, where each W^{a} is independently selected from the group consisting of hydroxy, halogen, (1-2)alkoxy and (1-2C)haloalkyl;
m is 0;
o is 0 or 1 and Y is selected from the group consisting of (1-3C)alkylene or phenylene, where any (1-3C)alkylene or phenylene in Y is optionally substituted with one or more Y^{a}, where each Y^{a} is independently selected from the group consisting of hydroxy, halogen, (1-2)alkoxy and (1-2C)haloalkyl; and
p is 1 and Z is —NR^{z}—, where R^{z} is hydrogen.

9. The compound, pharmaceutically acceptable salt, hydrate or solvate for use according to any preceding claim, wherein L¹ has a structure according to any one of the following:

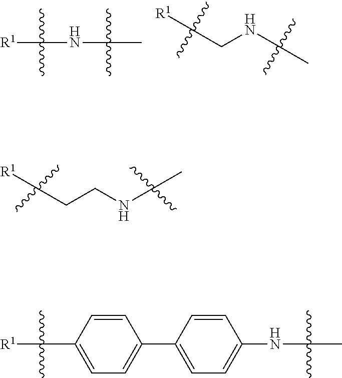

10. The compound, pharmaceutically acceptable salt, hydrate or solvate for use according to any preceding claim, wherein L² is selected from the group consisting of absent and (1-2C)alkylene, where any (1-2C)alkylene in L² is optionally substituted with one or more substituents selected form the group consisting of hydroxy, halogen and (1-2C)haloalkyl.

11. The compound, pharmaceutically acceptable salt, hydrate or solvate for use according to any preceding claim, wherein
R¹ is selected from the group consisting of N, CR², phenyl and cyclohexyl, where any phenyl or cyclohexyl in R¹ is optionally substituted with one or more R³;
each R³ is independently selected from the group consisting of hydroxy, halogen, (1-4C)alkyl, (1-4C)haloalkyl and —OR^{3a}, where any (1-4C)alkyl or (1-4C)haloalkyl in R³ is optionally substituted with one or more R^{3c};
R² is selected from the group consisting of hydrogen, and (1-3C)alkyl, where any (1-4C)alkyl in R² is optionally substituted with one or more R^{2c}.

12. The compound, pharmaceutically acceptable salt, hydrate or solvate for use according to any preceding claim, wherein R¹ has a structure according to any one of the following:

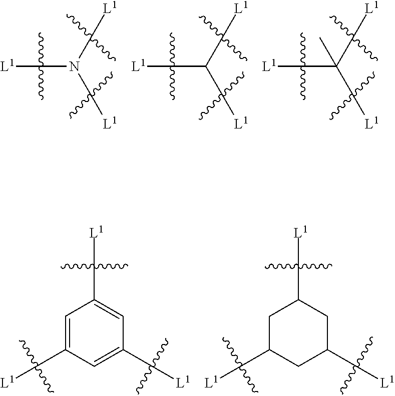

13. The compound, pharmaceutically acceptable salt, hydrate or solvate for use according to any preceding claim, wherein R^{5b} and R^{5c} are each independently selected from the group consisting of hydrogen, (1-3C)alkyl and (1-3C)haloalkyl.

14. The compound, pharmaceutically acceptable salt, hydrate or solvate for use according to any preceding claim, wherein M is selected from the group consisting of Zn²⁺, Cu²⁺, Mn²⁺ and Fe²⁺.

15. The compound, pharmaceutically acceptable salt, hydrate or solvate for use according to any preceding claim, wherein the compound of Formula I, or a pharmaceutically acceptable salt, hydrate or solvate thereof, is in further combination with a source of Q, wherein Q is an anion selected from the group consisting of spherical monoanionic anions, trigonal planar anions, dianionic tetrahedral anions, trianionic tetrahedral anions, dianionic octahedral anions and trianionic octahedral anions.

16. The compound, pharmaceutically acceptable salt, hydrate or solvate for use according to claim 15, wherein Q is an anion selected from the group consisting of dianionic tetrahedral oxoanions and trianionic tetrahedral oxoanions.

17. The compound, pharmaceutically acceptable salt, hydrate or solvate for use according to claim 15 or 16, wherein Q is sulfate (SO₄²⁻), phosphate (PO₄³⁻) or organophosphate (such as monophenylphosphate).

18. The compound, pharmaceutically acceptable salt, hydrate or solvate for use according to any preceding claim, wherein the compound of Formula I has a structure according to any one of the following: 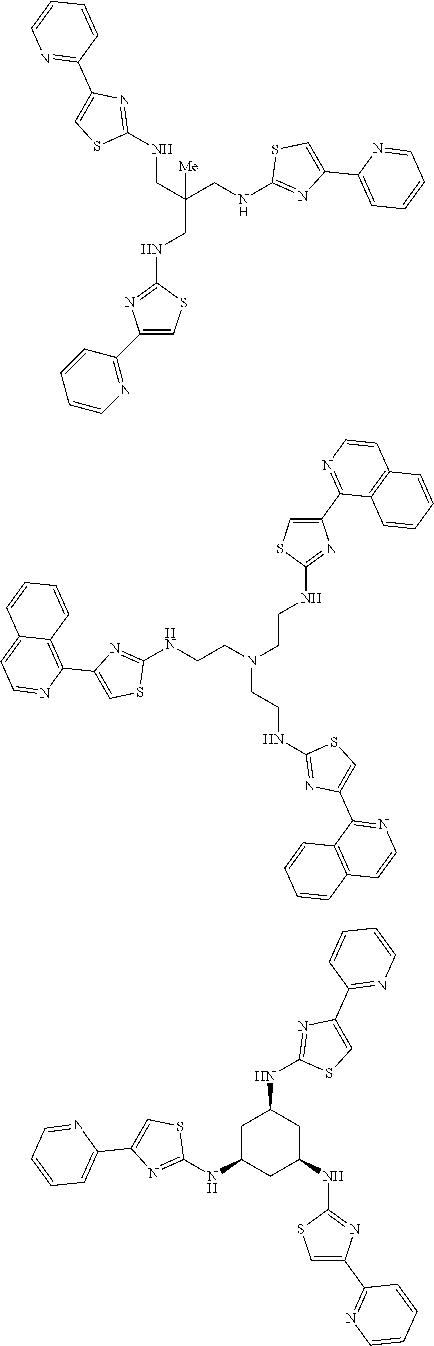 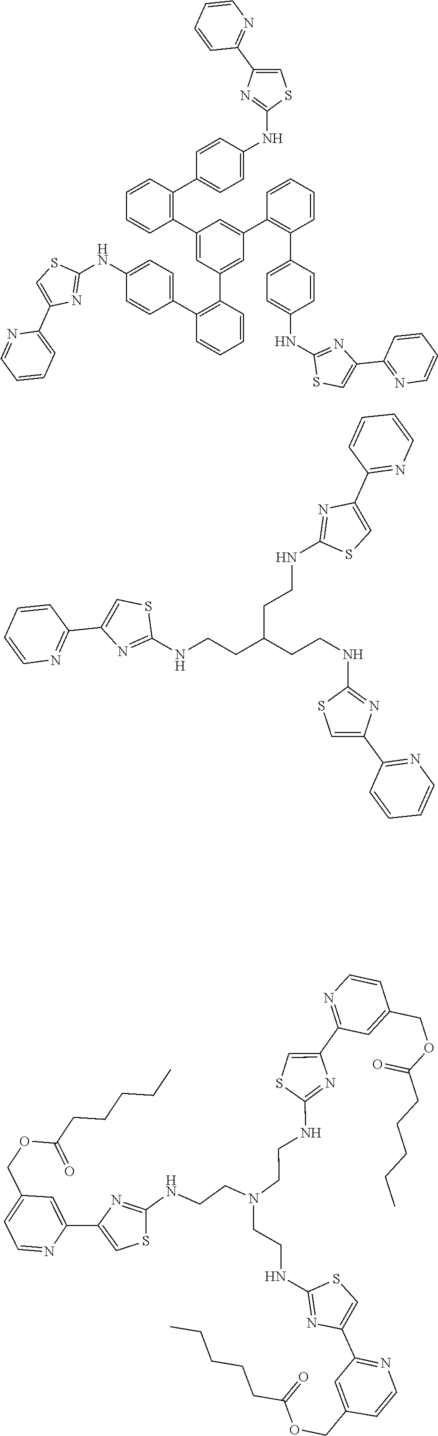 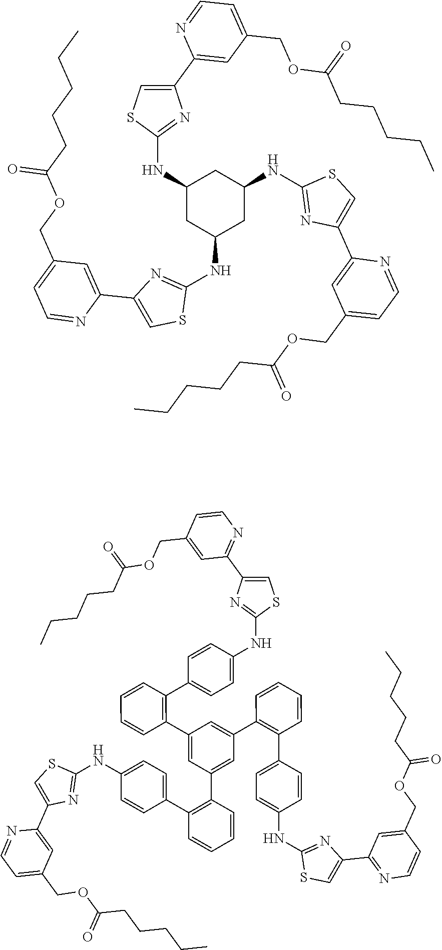 [SMILES: CCCCCC(=O)OCc1ccnc(-c2csc(N[C@H]3C[C@@H](Nc4nc(-c5cc(COC(=O)CCCCC)ccn5)cs4)C[C@@H](Nc4nc(-c5cc(COC(=O)CCCCC)ccn5)cs4)C3)n2)c1 ; CCCCCC(=O)OCc1ccnc(-c2csc(Nc3ccc(-c4ccccc4-c4cc(-c5ccccc5-c5ccc(Nc6nc(-c7cc(COC(=O)CCCCC)ccn7)cs6)cc5)cc(-c5ccccc5-c5ccc(Nc6nc(-c7cc(COC(=O)CCCCC)ccn7)cs6)cc5)c4)cc3)n2)c1] 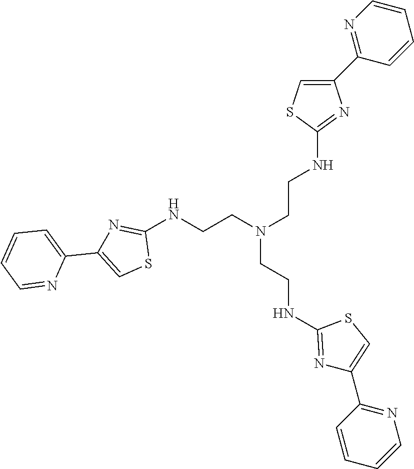 [SMILES: c1ccc(-c2csc(NCCN(CCNc3nc(-c4ccccn4)cs3)CCNc3nc(-c4ccccn4)cs3)n2)nc1]
or a pharmaceutically-acceptable salt, hydrate and/or solvate thereof.

19. The compound, pharmaceutically acceptable salt, hydrate or solvate for use according to any preceding claim, wherein the compound of Formula I has the structure: 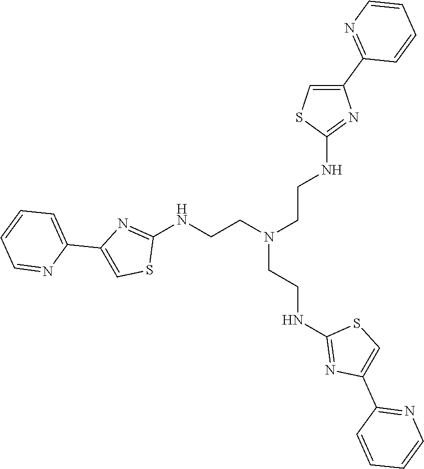 [SMILES: c1ccc(-c2csc(NCCN(CCNc3nc(-c4ccccn4)cs3)CCNc3nc(-c4ccccn4)cs3)n2)nc1]
or a pharmaceutically-acceptable salt, hydrate and/or solvate thereof.

20. A compound having a structure according to Formula II shown below, or a pharmaceutically acceptable salt, hydrate or solvate thereof, for use as a medicament: 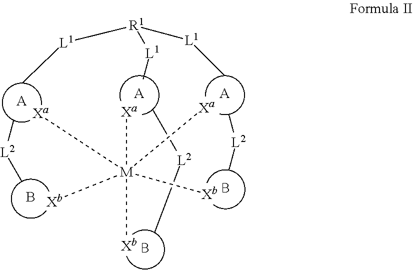
wherein
M, R¹, L¹, A, X^{a}, L², B, X^{b} and any associated subgroups are as defined in any preceding claim.

21. The compound, pharmaceutically acceptable salt, hydrate or solvate for use according to claim 20, wherein the compound of Formula II, or a pharmaceutically acceptable salt, hydrate or solvate thereof, is in further combination with a source of Q as defined in any one of claims 15, 16 and 17.

22. The compound, pharmaceutically acceptable salt, hydrate or solvate for use according to claim 20 or 21, wherein the compound of Formula II has a structure according to any one of the following: 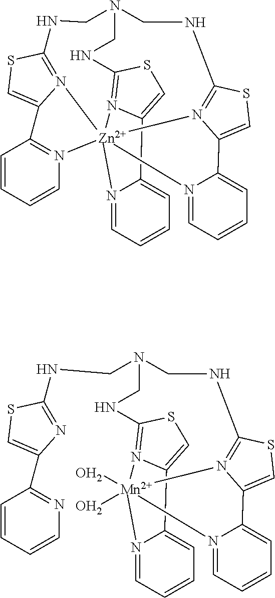
or a pharmaceutically-acceptable salt, hydrate and/or solvate thereof.

23. A compound having a structure according to Formula III shown below, or a pharmaceutically acceptable salt, hydrate or solvate thereof, for use as a medicament: 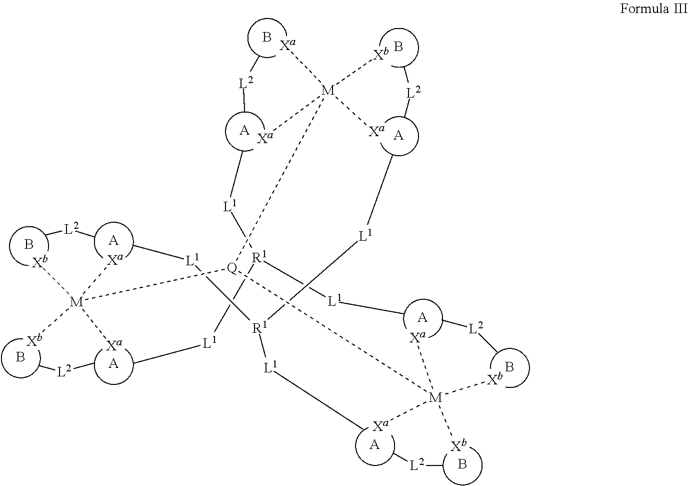
wherein
M, Q, R¹, L¹, A, X^{a}, L², B, X^{b} and any associated subgroups are as defined in any one of claims 1-19.

24. The compound, pharmaceutically acceptable salt, hydrate or solvate for use according to claim 23, wherein the compound of Formula III has a structure according to any one of the following: 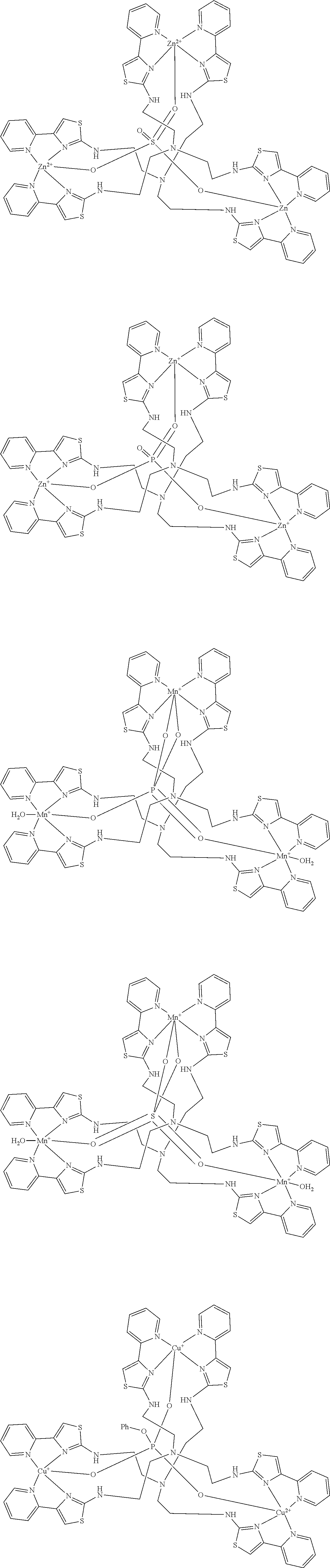
or a pharmaceutically-acceptable salt, hydrate and/or solvate thereof.

25. The compound, pharmaceutically acceptable salt, hydrate or solvate for use according to any preceding claim, wherein the medicament is for the treatment of a proliferative disorder (e.g. cancer).
